(19) United States
(12) Patent Application Publication
(10) Pub. No.: US 2003/0207270 A1
(43) Pub. Date: Nov. 6, 2003

(54) Phytomics: a genomic-based approach to herbal compositions

(76) Inventors: Patrick C. Kung, Cambridge, MA (US); Konan Peck, Taipei (TW); Yun-Shien Lee, Keelung (TW); Yuh-Pyng Sher, Shijr Jen (TW); Yung-Chi Cheng, Woodbridge, CT (US)

Correspondence Address: MORGAN LEWIS & BOCKIUS LLP, 1111 PENNSYLVANIA AVENUE NW, WASHINGTON, DC, 20004, US

(21) Appl. No.: 10/030,453

(22) PCT Filed: Mar. 9, 2001
(86) PCT No.: PCT/US01/07608

(51) Int. Cl. C12Q 1/68; G06F 19/00; G01N 33/48; G01N 33/50

(52) U.S. Cl. 435/6; 702/20

(57) ABSTRACT

The present invention provides the tools and methodologies necessary to guide the standardization of herbal compositions, to determine which specific components of an herbal composition are responsible for any particular biological activity, to predict the biological activities of a particular herbal composition, to determine the relatedness of herbal compositions, and for the development of improved herbal therapeutics. This invention provides the tools and methodologies for creating, maintaining, improving and utilizing Herbal BioResponse Arrays (HBR Arrays), wherein the HBR Arrays constitute data sets associated with particular herbal compositions. The HBR Arrays of the present invention contain gene expression profiles and may also include information on the plant-related parameters of the herbal constituents, marker information collected following the exposure of a biosystem to the herbal composition, and biological response information collected following the exposure of a biosystem to the herbal composition.

### FIELD OF THE INVENTION

This invention relates to herbal compositions. More specifically, this invention provides tools and methodologies for improving the selection, testing, quality control and manufacture of herbal compositions, and to help guide the development of new herbal compositions and identify novel uses of existing herbal compositions.

### BACKGROUND OF THE INVENTION

All publications and patent applications herein are incorporated by reference to the same extent as if each individual publication or patent application was specifically and individually indicated to be incorporated by reference.

Herbal medicine has been in use for centuries by people of Asia and Europe. In the United States (US), herbs have become commercially valuable in the dietary supplement industry as well as in holistic medicine. Approximately one third of the US population has tried some form of alterative medicine at least once (Eisenberg et al., 1993, N. Engl. J. Med. 328:246-252).

Botanicals, including herbs, have also become a focal point for the identification of new active agents to treat diseases. Active compounds, derived from plant extracts, are of continuing interest to the pharmaceutical industry. For example, taxol is an antineoplastic drug obtained from the bark of the western yew tree. It is estimated that approximately 50 percent of the thousands of drugs commonly used and prescribed today are either derived from a plant source or contain chemical imitations of a plant compound (Mindell, E. R., 1992, Earl Mindell's Herb Bible, A Fireside Book).

Currently, a number of medicinal formulations, food supplements, dietary supplements and the like contain herbal components or extracts from herbs. Herbal medicines have been used for treating various diseases of humans and animals in many different countries for a very long period of time (see, e.g., I. A. Ross, 1999, Medicinal Plants of the World, Chemical Constituents, Traditional and Modern Medicinal Uses, Humana Press; D. Molony,. 1989, The American Association of Oriental Medicine's Complete Guide to Chinese Herbal Medicine, Berkley Books; Kessler et al., 1996, The Doctor's Complete Guide to Healing Medicines, Berkley Health/Reference Books); Mindell, supra).

Herbal Medicines. There are many branches of herbal medicine around the world, such as Ayurveda, Unani, Sida and Traditional Chinese Medicine (TCM). While modern Western medicine typically consists of administering a single chemical entity capable of intervening specific biochemical pathways, each formula of TCM typically contains hundreds of chemical entities from several herbs which are designed to interact with multiple targets in the body in a coordinated manner. Although empirical practice contributed in a significant way to the herbal composition and prescription of these ancient herbal medicines, they are also supported, to a varying degree, by a set of theories which all are distinct from that of modern Western medicine in terms of anatomy, pharmacology, pathology, diagnosis treatment, etc. Among the different herbal medicine fields, TCM has developed a more complete set of theories over several centuries which have been well documented and practiced by local physicians caring for a huge population (>1.3 billion people) in greater China and in East Asia including Korea and Japan.

Western medicine generally uses purified compounds, either natural or synthetic, mostly directed towards a single physiological target. However, the compositions used in TCM are usually composed of multiple herbs and compounds which are aimed at multiple targets in the body based on unique and holistic concepts. TCM mainly used processed crude natural products, with various combinations and formulations, to treat different conformations resulting in fewer side effects. The great potential of TCM has yet to be realized for the majority of the world's people.

The herbs in a typical TCM prescription are assigned roles as the principal herb and the secondary herbs, including assistant, adjuvant and guiding herbs. The principal herb produces the leading effect in treating the cause or the main symptom of a disease. An assistant herb helps to strengthen the effect of the principal herb and produces leading effects in the treatment of the accompanying symptoms. There are three types of adjuvant herbs: 1) those that enhance the therapeutic effects of the principal and assistant herbs or treat tertiary symptoms, 2) those that reduce or eliminate the toxicity and other side effects of the principal and the assistant herbs and 3) those which act on complementary target tissues not specifically affected by the principal herb. A guiding herb directs the effect of other herbs to the affected site and/or coordinates and mediates the effects of the other herbs in the prescription or formulation. In contrast to most of the herbal medicines or supplements that consist of one or more parts of a single plant, the intended effects of TCM are directed at multiple tissues.

For example, a well-known TCM recipe, “Ephedra Decoction” used for treating asthma is composed of ephedra, cinnamon twig, bitter apricot kernel and licorice. Ephedra, is the principal herb, which expels cold, induces diaphoresis and facilitates the flow of the Lung Qi to relieve asthma, the main symptom. Cinnamon twig, as the assistant herb, enhances ephedra's induction of diaphoresis and warms the Channels to ensure the flow of Yang Qi for reducing headache and pantalgia. Bitter apricot kernel, as the adjuvant herb, facilitates the adverse flow of the Lung Qi and strengthens the asthma relief by ephedra. Licorice as the guiding herb moderates the effects of both ephedra and cinnamon to ensure a homeostasis of the vital Qi. While each of the four herbs clearly exhibits its respective activity, they complement as well as supplement each other when they are combined. In practice, the principal herb can be prescribed with one or more secondary herbs, depending on the symptoms at a patient's presentation (Prescriptions of Traditional Chinese Medicine, Chapter One, pp10-16, E. Zhang, editor in Chief, Publishing House, Shanghai University of Traditional Chinese Medicine, 1998).

The main theories of TCM that guide the treatment of sickness with herbal medicine and other means, such as acupuncture, are 1) the theory of Yin and Yang, 2) the theory of Five Elements, 3) the theory of Viscera and Bowels, 4) the theory of Qi, Blood and Body Fluid, and 5) the theory of Channels and Collaterals.

In TCM, the first important aspect of making the proper diagnosis is to ascertain whether the disease is Yin or Yang. For example, those patients who have a fever, are thirsty, constipated or have a rapid pulse condition are of Yang character. Those individuals who have an aversion to cold, are not thirsty, and diarrhea and a slow pulse condition are of Yin character. The property, flavor and function of herbs can also be classified according to Ying and Yang theory. For example, herbs of cold and cool nature belong to Ying, while herbs which are warm and hot in nature belong to Yang. Herbs with sour, bitter and salty flavor belong to Ying, while herbs with pungent, sweet and bland flavor belong to Yang. Herbs with astringent and subsiding function belong to Yin, while herbs with dispersing, ascending and floating function belong to Yang. In TCM, the principles of treatment are based on the predominance or weakness of Yin and Yang. Herbs are prescribed according to their property of Ying and Yang and their function for restoring the imbalance of the Ying and Yang. In so doing, the benefit of treatment is achieved.

According to the theory of Five Elements there are five basic substances that constitute the material world (i.e., wood, fire, earth, metal and water). In TCM, this theory has been used to explain the physiology and pathology of the human body and to guide clinical diagnosis and treatment. Herbal physicians have applied the laws of generation, restriction, subjugation and reverse restriction of the five elements to work out many effective and specific treatment regimens, such as reinforcing earth to generate metal (strengthening the function of the spleen to benefit the lung), replenishing water to nourish wood (nourishing the essence of the kidney to benefit the liver), supporting earth to restrict the wood (supplementing the function of the spleen to treat the hyperactivity of the liver), and strengthening water to control fire (replenishing the essence of the kidney to treat hyperactivity of the heart). Specifically, the property of some herbs is assigned to each of the five Elements for the purposes of guiding the prescription of a TCM recipe.

In TCM, the internal organs of the human body are divided into three groups: five Viscera (the Heart, the Liver, the Spleen, the Lung and the Kidney), Six Bowels (the Gall Bladder, the Stomach, the large Intestine, the Small Intestine, the Urinary Bladder, and the Triple Warmer), the Extraordinary Organs (the Brain, the Medulla, the Bone, the Blood Vessel, the Gall Bladder, and the Uterus). In TCM, the Viscera or the Bowel are not only anatomic units, but are also concepts of physiology and pathology about interactions between different organs. For example, the heart also refers to some of the mental functions and influence functions of blood, hair, tongue and skin. Ying-Yang and the Five Elements influence the interactions among these Viscera, Bowels and Organs. The complexity of interplay of the theories is used to explain the pathology of diseases to which herbs are prescribed, as discussed below.

The prescription of herbal medicine in TCM starts with the diagnosis, which consists of four main items: interrogation, inspection, auscultation and olfaction, pulse taking and palpation. During the interrogation phase, much information is gathered, including the characteristics of the main symptoms. For instance, if the main symptom is characterized by dull pain of epigastric region, which may be relieved by warming and pressing, this suggests the insufficiency of the Spleen-Yang. Soreness and weakness of the loins and knees, intolerance of coldness with cold extremities manifests a weakness of the Kidney-Yang. During inspection, observations are made for vitality, skin color and the general appearance and the condition of the tongue. For example, a pale complexion corresponds internally to the Lung of autumn, whose Qi is dry. This may occur when Yang Qi is lacking and the circulation of Qi and blood is impeded, or when the coldness in the channels and collaterals causes them to contract.

In TCM, it is from Qi, blood and body fluid that come energy needed by the Viscera and Bowels, Channels and Collaterals, tissues and other organs for carrying-out their physiological functions; and on which the formation and metabolism of Qi, blood and body fluid depend. Prescriptions of TCM consider the herbal effects on Qi and blood for treatments.

TCM holds that Channels, Collaterals and their subsidiary parts are distributed over the entire body. It is through them that herbs exert influence on pathological targets and achieve the improvement of sickness. For example, ephedra acts on the Channels of the Lung and Urinary Bladder so as to induce sweat for relieving asthma and promoting diuresis. As noted above, clinical applications of acupuncture are also guided by the theory of Channels and Collaterals.

In summary, while the nature or property of each herb in TCM may be assigned as Yin or Yang, and to one of the Five Elements, they act through Channels and Collaterals and are mediated via Qi, Blood and Fluid to yield therapeutic effects on targets, such as Viscera and Bowels. Pathogenic factors may be disguised as decoy through the very same systems of Channels and Collaterals to adversely affect the functions of Viscera and Bowels and thus cause sickness.

From the foregoing discussion, it is clear that the TCM terminology is as much of a philosophical concept as an anatomical one. For example, the Heart represents a host of tissues, organs or systems in the body that contribute to a function described in TCM. Thus, the concept of the Heart requires a multiple dimension data set to describe each concept of TCM. Once this is accomplished, a molecular holistic medicine can be developed.

U.S. Regulatory Process. In the US, dietary supplements (such as botanical products, vitamins and minerals, amino acids and tissue extracts) are regulated under the Dietary Supplement Health and Education Act of 1994 (the DSHE Act). This Act removed the ingredients of dietary supplements from regulation as food additives under the Federal Food, Drug, and Cosmetic Act: In addition, the DSHE Act requires that The Food and Drug Administration (FDA) bear the burden of proof that a marketed dietary supplement presents a serious or unreasonable risk under the conditions of use on the label or as commonly consumed. Thus, there are currently no federal regulations that establish specific criteria for purity, identification and manufacturing procedures for dietary supplements. In addition, few published papers on herbal quality have resulted from the establishment of the Office of Alternative Medicine by Congress in 1992 (Angell et al., 1998, N. Engl. J. Med. 339:839-841).

At the present time, the FDA must approve each one of the chemical entities in a drug composition or cocktail, and then clinical trials must be undertaken so as to obtain separate FDA approval for marketing the drug. This process is extremely tedious and costly. A molecular holistic medicine may require a less arduous evaluation since the previous use of a particular herbal composition as a botanical drug permits clinical trials with multiple chemicals at the outset (i.e., clinical trials using the herbal composition or specific components of the herbal composition). Recently, the FDA has approved the testing of some herbal medicines in clinical trials as botanical drugs (FDA Guidance on Botanical Drugs, April, 1997). While these events represent a positive development for health care in general, it also raises important issues regarding the formulation, manufacturing and quality control of herbal medicines and dietary supplements, including the traditional Chinese medicines.

The multitude of relevant biological responses induced by the multiple chemicals in herbs are not currently available and will be increasingly important to support marketing approval by the FDA.

Herbal-based industries are coming under increasing pressure to upgrade their current practices (see, e.g., Angell et al., supra). The need to apply scientific testing to the preparation and administration of herbal medicines and food supplements has been highlighted by several recent reports of toxicity resulting from ingesting herb-based formulations. For example, one patient who took an herbal-based dietary supplement experienced digitalis toxicity (Slifman et al., 1998, N. Engl. J. Med. 339:806-811). It was subsequently determined that the herb ingredient labeled as plantain in the supplement was actually contaminated with Digitalis lanata, an herb known to contain at least 60 cardiac glycosides. In another instance, an herbal preparation was found to be the cause of chronic lead intoxication in a patient (Beigel et al., 1998, N. Engl. J. Med. 339:827-830). This is not a completely unexpected occurrence since contamination of traditional Asian herbal remedies by lead and other heavy metals is well documented (Woolf et al., 1994, Ann. Intern. Med. 121:729-735).

Characterization of Botanicals. It is well known that the genetic identity (e.g., genera, species, cultivar, variety, cone), age of herbal growth, harvest time, the specific plant part utilized, processing method, geographical origin, soil type, weather patterns, type and rate of fertilizer, and other growth factors have a great impact on the particular chemical composition of any particular herb “harvested” from any particular area.

Increasing numbers of various types of tests have been instituted to assure the consistent quality of herbs used in medicine and as dietary supplements; including inspections at the macro- and microscopic levels as well as a variety of chemical analyses. Recently, high performance liquid chromatography (HPLC) profile of marker molecules in an herbal extract has become one reference standard. However, there are problems with this approach, including that some of the bioactive molecules may not adsorb UV or the visible lights for HPLC detection, and the amount of a chemical is not necessarily proportional to its biological potency. For these reasons, herbal manufacturers resort to a practice of mixing raw herbs from different sources to minimize chemical variations.

Mass spectrometry (MS) is an analytical method for determining the relative masses and relative abundances of components of a beam of ionized molecules or molecular fragments produced from a sample in a high vacuum. MS, unlike HPLC, is not optical density-dependent. In practice it is used in conjunction with HPLC or capillary electrophoresis (CE): the HPLC separates the chemicals and the MS then can be used to identify what they are. Commercial systems are available which integrate MS and HPLC for biological uses. Mass spectrometry is limited to samples that are gaseous or volatile at low pressure, or that can be so rendered by derivatization.

These steps are no longer adequate. Recent publications report a greater variation in the quality of herbs by specific suppliers, and the difficulty of providing biological equivalence of herbal extracts. Furthermore, the correlation between safety and efficacy and chemicals in an herb is not well defined in most cases. Recently, in response to complaints from consumer groups and regulatory agencies (Federal Register, Feb. 6, 1997, Volume 62, No. 25, Docket No. 96M-0417; cGMP in Manufacturing, Packing or Holding Dietary Supplements, Proposed Rules), some herbal manufacturers have begun to implement Good Manufacturing Practice (GMP) which requires stringent controls at all levels.

Chemical and spectroscopic methods have been used to characterize the components of herbal medicines and food supplements. For example, three new hederagenin-based acetylated saponins were isolated from the fruits of Gliricidia sepium using these two methods (Kojima et al., 1998, Phytochemistry 48(5):885-888). The botanical sources of Chinese herbal drugs in a number of commercial samples were inferred by comparing the contents of some characteristic constituents which were analyzed with high-performance chromatography (HPLC) or capillary electrophoresis (CE) (Shuenn-Jyi Sheu, 1997, Journal of Food and Drug Analysis 5(4):285-294). For example, the ratio of ephedrine/pseudoephedrine was used as a marker to differentiate Ephedra intermedia from other species; total alkaloid contents were used to distinguish between species of Phellodenldron; and the contents of ginsenosides were used to differentiate between species of Panax. However, these methods do not provide a direct measurement of the effect of the various herbs on the molecular, physiological or morphological responses following human treatment with the herbs.

Using gas chromatography-mass spectrometry and atomic-absorption methods, the California Department of Health Sciences, Food and Drug Branch, recently tested Asian medicines obtained from herbal stores for contaminants (R. J. Ko, 1998, N. Engl. J. Med. 339:847). Of the 260 products they tested, at least 83 (32 percent) contained undeclared pharmaceuticals or heavy metals, and 23 had more than one adulterant. Using high-performance liquid chromatography, gas chromatography, and mass spectrometry, a commercially available combination of eight herbs (PC-SPES), was found to contain estrogenic organic compounds (DiPaola et al., 1998, N. Engl. J. Med. 339:785-791). The researchers concluded that PC-SPES has potent estrogenic activity and that prostate cancer patients that took PC-SPES could confound the results of standard therapies and may experience clinically significant adverse effects. Gas chromatography data was also collected for different samples of the traditional Chinese medicine ‘wei ling xian’ and correlated to the antiinflammatory activity of the samples (Wei et al., Study of chemical pattern recognition as applied to quality assessment of the traditional Chinese medicine “wei ling xian,” Yao Hsueh Pao 26(10): 772-772 (1991)). This study did not provide relevant HBR Array data, such as time course, dose dependent response, control samples to substantiate the differential power of the biomarkers, nor it utilize a reiterative type of data construction process to establish a comprehensive database for characterizing effects of the herbal composition.

Changes in protein levels have also been used to characterize the effects of herbal compositions or specific components of herbs. For example, the production of granulocyte colony-stimulating factor (G-CSF) from peripheral blood mononuclear cells was found to vary depending on which specific Chinese herb was added to the culture (Yamashiki et al., 1992, J. Clin. Lab. Immunol. 37(2) 83-90). Expression of interleukin-1 alpha receptors was markedly up regulated in cultured human epidermal keratinocytes treated with Sho-saiko-to, the most commonly used herbal medicine in Japan (Matsumoto et al., 1997, Jpn. J. Pharmacol. 73(4):333-336). The expression of Fc gamma 11/111 receptors and complement receptor 3 of macrophages were increased by treatment with Toki-shakuyakusan (TSS) (J. C. Cyong, 1997, Nippon Yakurigaku Zasshi 110(Suppl. 1):87-92). Tetrandrine, an alkaloid isolated from a natural Chinese herbal medicine, inhibited signal-induced NF-kappa B activation in rat alveolar macrophages (Chen et al., 1997, Biochem. Biophys. Res. Commun. 231(1):99-102). The herbs Sairei-to, alismatis rhizoma (Japanese name “Takusha”) and hoelen (Japanese name “Bukuryou”) inhibited the synthesis and expression of endothelin-1 in rats with anti-glomerular basement membrane nephritis (Hattori et al., 1997, Nippon Jinzo Gakkai Shi 39(2):121-128).

The increase or decrease in mRNA levels has also been used as an indicator of the effect of various herbs and herbal components. Intraperitoneal injection of Qingyangshen (QYS), a traditional Chinese medicine with antiepileptic properties, and diphenylhydantoin sodium reduced alpha- and beta-tublin mRNAs and hippocampal c-fos mRNA induction during kainio acid-induced chronic seizures in rats (Guo et al., 1993, J. Tradit. Chin. Med. 13(4):281-286; Guo et al., 1995, J. Tradit. Chin. Med. 15(4):292-296; Guo et al., 1996, J. Tradit. Chin. Med. 16(1):48-51). Treatment of cultured human umbilical vein endothelial cells (HUVECs) with the saponin astragaloside IV, a component purified from Astragalus membranaceus, decreased plasminogen activator inhibitor type I (PAI-1) specific mRNA expression and increased tissue-type plasminogen activator (t-PA) specific mRNA (Zhang et al., 1997, J. Vasc. Res. 34(4):273-280). One component isolated from the root of Panax ginseng was found to be a potent inducer of interleukin-8 (IL-8) production by human monocytes and by the human monocytic cell line THP-1, with this induction being accompanied by increased IL-8 mRNA expression (Sonoda et al., 1998, Immunopharmacology 38:287-294).

Recent advances in nucleic acid microarray technology enable massive parallel mining of information on gene expression. This process has been used to study cell cycles, biochemical pathways, genome-wide expression in yeast, cell growth, cellular differentiation, cellular responses to a single chemical compound, and genetic diseases, including the onset and progression of the diseases (M. Schena et al., 1998,TIBTECH 16:301). Because cells respond to the micro-environment changes by changing the expression level of specific genes, the identities of genes expressed in a cell determine what the cell is derived of and what biochemical and regulatory systems are involved, among other things (Brown et al. 1999, Nature genet., 21 (1) supplement:33): Thus, cellular gene expression profiles portray the origin, the present differentiation of the cell, and the cellular responses to external stimulants. No researchers to date, if any, have attempted to apply these new technologies to study the molecular effects of whole herbal treatments and supplements.

Some researchers have attempted to characterize the effects of the major active constituents isolated from selected herbs. For example, treatment of HUVECs with notoginsenoside R1 (NR1), purified from Panax notoginseng, resulted in a dose- and time-dependent increase in TPA synthesis (Zhang et al., 1994, Arteriosclerosis and Thromobosis 14(7):1040-1046). Treatment with NR1 did not change urokinase-type plasminogen activator and PAI-1 antigen synthesis, nor did it effect the deposition of PAI-1 in the extracellular matrix. TPA mRNA increased as much as twofold when HUVECs were treated with NR1, whereas expression of PAI-1 -specific mRNA was not significantly affected by NR1. Since most studies on P. notoginseng have involved its mixture with other herbs, the researchers noted that it was difficult to assess how their results relate to the situation in vivo when is used therapeutically in humans (Id., at 1045, second column, first paragraph). In addition, since the researchers only studied one major component of the herb, it is not possible to ascertain the molecular effect of the whole herb or the interactions among components of the herb from this study.

Dobashi et al. (1995, Neuroscience Letters 197:235-238) studied the effect of two of the main components of saiko agents, a Chinese herbal drug used to treat nephrotic syndrome, bronchial asthma and chronic rheumatoid arthritis. Administration of SS-d increased plasma adrenocorticotropin (ACTH) levels; proopiomelanocortin mRNA levels in the anterior pituitary and the CRF mRNA level in the rat hypothalamus in a dose dependent manner. In contrast, treatment with SS-a failed to affect the levels of these molecular markers. While this study indicates that administration of SS-d may have an important role in saiko agents-induced CRF release and CRF gene expression in rat hypothalamus, it fails to address the molecular effect of the herbal medication as a whole.

Kojima et al. (1998, Biol. Pharm. Bull. 4:426-4228) describe the utilization of differential display of mRNA to isolate and identify genes transcriptionally regulated in mouse liver by sho-saiko-to, an herbal medicine used for treating various inflammatory diseases in Japan. These researchers limited their study to the use of mRNA differential display techniques in investigating the molecular mechanisms of herbal medicine. It also failed to address effects in multiple organs of treated animals and did not provide any guidance for quality control, new use, and standardization of effects. In addition, the study failed to analyze the individual components of the herb and compare the individual results with the results obtained using the whole herbal mixture.

Ma Ji et al. (1998; Chinese Medical Journal 111(1): 17-23) investigated the therapeutic effect of the herb Astragali membranaceus on sodium and water retention in rats experiencing aortocaval fistula-caused experimental congestive heart failure. Chronic heart failure rats with and without Astraglia treatment were compared for changes in various morphological characteristics (e.g., body weight, serum sodium concentration); physiological characteristics (e.g., mean arterial pressure, heart rate, hematocrit and plasma osmolality); mRNA expression levels (e.g., hypothalamic, arginine vasopressin (AVP), AVP V₁a receptor, renal AVP V₂ receptor, aquaporin-2 (AX-2)) and protein excretion (e.g., plasma atrial monophosphate peptide (ANP) and urinary cyclic guanidino monophosphate (cGMP)). The researchers found that treatment with Astraglia improved cardiac and renal functions, partially corrected abnormal mRNA expressions of the AVP system and AQP2, and improved the renal reaction to ANP. This study did not address using the collected data to guide the development of new formulations or for elucidating the synergistic or other interactions among various herbs in a formula, or validate the differential power of the effects for quality control purposes.

As shown by the above review of relevant scientific articles, molecular-based technology has not been used to explore and validate cellular and molecular responses in biological systems that are treated or challenged with multiple chemicals at the same time, such as herbal medicines and TCM. Furthermore, these recent advances have not been integrated with other technologies and methods to produce a process for the systematic exploration of biological effects of herbal medicines and TCM.

### SUMMARY OF THE INVENTION

This invention provides the tools and methodologies for creating, maintaining, improving and utilizing Herbal BioResponse Arrays (HBR Arrays), wherein the HBR Arrays constitute data sets associated with particular herbal compositions. The HBR Arrays of the present invention may include information on the plant-related parameters of the herbal constituents, marker information collected following the exposure of a biosystem to the herbal composition, and biological response information collected following the exposure of a biosystem to the herbal composition.

The present invention provides the tools and methodologies necessary for establishing standardized HBR Arrays for particular herbal compositions, wherein the standardized HBR Arrays are used as benchmarks by which to evaluate batches of similar or different herbal compositions. The present invention further provides the tools and methodologies necessary to update and maintain the standardized HBR Arrays. Particular embodiments of the present invention involve iterative processes whereby data for additional batches of the herbal composition, additional plant-related data, additional marker information, and/or additional BioResponse information is periodically added to the standardized HBR Arrays. Thus, the present invention provides the tools and methodologies for creating, maintaining, updating and using HBR Arrays on an on going basis.

The present invention provides the tools and methodologies necessary to guide the standardization of herbal compositions; to determine which specific components of herbal compositions are responsible for particular biological activities; to predict the biological activities of herbal compositions; for the development of improved herbal therapeutics; for adjusting or modifying an herbal composition; for measuring the relatedness of different herbal compositions; for identifying specific molecules in the batch herbal composition which retain the desired biological activity; for determining which herbal components of a known herbal composition can be eliminated from the known herbal composition while maintaining or improving the desired biological activity of the known herbal composition; for identifying new uses and previously unknown biological activities for the batch herbal composition; and for using the predicted biological activity of the batch herbal composition to aid in the design of therapeutics which include herbal components and synthetic chemical drugs, including the design of therapeutics using the methods of combinatorial chemistry.

More specifically, the present invention provides methods of establishing standardized Herbal BioResponse Arrays (HBR Arrays) for herbal compositions, wherein the methods comprise:

a) selecting a characterized herbal composition;

b) exposing a biosystem to a batch of the characterized herbal composition and collecting data on two or more markers, wherein one of the markers is a change in gene expression determined through the use of a nucleic acid microarray, produced by the steps comprising:

i) producing a cell banking system;

ii) profiling the gene expression pattern of cells from the cell banking system before and after exposure to the herbal composition;

iii) selecting as markers those genes whose expression levels are changed by exposure to the herbal composition;

c) storing the marker data of step b) as a standardized HBR array.

The present invention further provides such methods which further comprise exposing a biosystem to one or more batches of the herbal composition, collecting the data on one or more BioResponses, and adding the collected BioResponse data to the standardized HBR Array for that herbal composition.

The present invention provides methods of evaluating herbal compositions, wherein the methods comprise exposing a biosystem to a batch of the herbal composition and collecting data on two or more markers, and comparing the collected marker data with a standardized HBR Array for the same or a substantially same herbal composition as that of the batch herbal compositions.

The present invention provides a system for predicting the biological activity of an herbal composition comprising:

1). a biosystem comprising one or more different types of cells, tissues, organs or in vitro assays;

2). a batch herbal composition;

3). two or more molecular markers;

4). a means for exposing the biosystem to the batch herbal composition and measuring the differential responses of the molecular markers;

5). a computer processor, including memory, for analyzing and storing the differential response measurements of the molecular markers so as to create an Herbal BioResponse Array (HBR Array) data set for the batch herbal composition;

6). a computer processor, including memory, for comparing the HBR Array of the batch herbal composition to one or more previously-stored HBR Arrays so as to predict the biological activity of the batch herbal composition, wherein the biological activities of the herbal compositions used to generate the one or more previously-stored HBR Arrays are known.

### BRIEF DESCRIPTION OF THE DRAWINGS

FIG. 1. FIG. 1 provides a schematic of the basic method steps for constructing a Standardized Herbal BioResponse Array (HBR Array) for any selected herbal composition. The figure is shown in its most basic form for ease of understanding. As discussed herein, each of the pathways of the schematic can be done iteratively. Furthermore, any information contained in one box can be used to guide decisions regarding gathering information for any other box. In this way, numerous feedback loops are also possible throughout the scheme.

FIG. 2. FIG. 2 provides a schematic of the basic method steps for constructing a an Herbal BioResponse Array (HBR-Array) for any batch herbal composition and for comparing this batch HRB Array to a selected subset of information from the Standardized HBR Array. The figure is shown in its most basic form for ease of understanding. As discussed herein, each of the pathways of the schematic can be done iteratively. Furthermore, any information contained in one box can be used to guide decisions regarding gathering information for any other box. In this way, numerous feedback loops are possible throughout the scheme.

FIG. 3. FIG. 3 provides a schematic of the basic method steps for establishing and using a major data set. The figure is shown in its most basic form for ease of understanding. As discussed herein, each of the pathways of the schematic can be done iteratively. Furthermore, any information contained in one box can be used to guide decisions regarding gathering information for any other box. In this way, numerous feedback loops are possible throughout the scheme.

FIG. 4. Western blot for various herbal compositions.

A. No herbal composition.

B. Huang Qing Tang A (HQT A) (0.2 mg/ml).

C. HQT A (4 mg/ml).

D. HQT B (0.2 mg/ml).

E. HQT B (4 mg/ml).

F. Scute (0.2 mg/ml).

G. Scute (4 mg/ml).

FIG. 5. TPLC for Paeonie lactiflora pallus.

FIG. 6. HPLC for Ziziphi fructus.

FIG. 7. FIG. 7 provides a schematic for establishing a bio-response data set for an herbal composition. The data set is based on differentially expressed gene induced by the herbal medicine for more than three different concentrations in a mammalian cell culture.

FIG. 8. FIG. 8 provides a schematic for establishing a characteristic expression profile database or HBR Array for an herbal medicine or a complex herbal preparation.

FIG. 9. FIG. 9 provides a schematic for identifying an unknown herbal composition. The expression profiles induced by the unknown herbal medicine are aligned with the expression profile database and statistical method is employed to score the possible identities of herbal medicines archived in the database.

FIG. 10. FIG. 10 provides a schematic for extracting signature genes for an herbal composition or a complex herbal preparation.

FIG. 11. FIG. 11 provides a schematic for extracting signature genes for individual chemical constituents in an herbal medicine or a complex herbal preparation.

FIG. 12. Clustered display of gene expression data from cells treated with three types of single-element herbal extracts (Cordyceps Sinensis Mycelium(CSM), ST024, ST117) with high and low concentrations (indicated with H and L, respectively).

(A) Cluster analysis was performed by the program “Cluster” (Eisen et al., 1999) with 492 selected genes (see text).

(B) Enlarged image of genes up-regulated by ST117 treatment but down-regulated by other herbal extract treatments. The clone ID and putative gene name are indicated.

(C) The clustering algorithm separated CSM, ST024 and ST117 into 3 distinct clusters. The distance between each cluster as displayed by the hierarchical dendrogram can be viewed as the difference between the expression profiles of the three herbal extracts treated cells.

FIG. 13.

(A) Pseudo-color encoded display of clustering results as calculated based on the selected 492 genes. The boxes in (A) indicate the positions of the three clusters of genes described above.

(B) Enlarged image of genes down-regulated by the CSM but up-regulated by the others.

(C) Genes up-regulated by all kinds of herbal treatments.

(D) Genes down-regulated by CSM and up-regulated by the others. The IMAGE clone ID and putative gene name are indicated.

FIG. 14. Clustered display of expression data from 2 batches of multi-element herbal preparations of the Huang Chin Tang (PHY906-303503 (#11) and PHY906-284003 (#12)) treated cells with high and low concentrations (indicated with H and L, respectively). The data were averaged based on three repeated experiments on three different dates. Cluster analysis was performed based on the selected 500 genes (see text). (B) The clustering algorithm separated #11-L, #11H and (#12-H and #12-L) into 3 distinct clusters. Distance between clusters or resemblance coefficient is indicated by the hierarchical clustering dendrogram.

FIG. 15. Enlarged image of (A) averaged and (B) individual gene expression levels measured by three independent experiments. Box1 encloses genes that were down regulated in #11-L treated cells but up regulated in others, Box2 encloses the genes that were up regulated by all the herbal treatments. Box3 enclosed the genes that showed no response by #11-L treatment but were down regulated by the others. Box4 encloses the genes highly down regulated by low concentration herbal treatments but show mild response at high concentration herbal treatments. The clone ID and putative gene name are indicated beside each gene.

FIG. 16. Classification of gene expression profiles in the cells treated by herbal medicines. Hierarchical clustering of (A) the data sets normalized with the expression data of the untreated control cells and (B) data sets standardized to have zero-mean and unit-variance. (C) The result of a non-hierarchical clustering by the self-organizing maps algorithm.

FIG. 17. Candidate class predictors for the classification of herbal medicines based on the gene expression profiles induced by the medicines. 50 class predictors with their expression profiles for discriminating #11 and #12 herbal preparations are shown in this figure. The IMAGE clone ID and putative gene name are indicated beside each gene.

FIG. 18. The gene expression profiles induced by a batch of a complex herbal preparation of five different concentrations. A 6×4 clustering of expression profiles is shown in (A), and the details of the gene expression profiles for the selected clusters are shown in (B).

FIG. 19. FIG. 19 illustrates how the expression profiles in FIG. 18 are categorized into three different groups for subsequent hamming distance calculation.

FIG. 20. FIG. 20 shows the analysis results of gene expression profiles induced by five batches of a complex herbal preparation. The numbers in the table are hamming distance. The smaller the distance, the more similar are the expression profiles.

FIG. 21. Shown in (A) is a table of integrated peak intensities of 4 chemical constituents in HPLC analyses of five batches of a complex herbal preparation. Two additional parameters, BG+B and BG/B are introduced t6 the table and a 6 parameter radial plot is shown in (B) to illustrate that one batch is more similar to a second batch #18 than to the other batches by the HPLC analysis.

FIG. 22. A display of the signature genes induced by a complex herbal preparation, the Huang Chin Tang, in Jurkat T cells.

FIG. 23. FIG. 23 illustrates the principle of identifying signature genes induced by individual chemical constituents in a mix of herbal medicines. The signature genes are those whose expression levels correlate with the amount of chemical constituents in the herbal medicine and that the correlation coefficient is larger than 0.99 or smaller than −0.99. (A) shows that the R value between the, gene and Glycyrrhizin was 0.998, and (B) shows that the gene whose, expression levels increase with the decrease of Wogonin has an R value of −0.997.

FIG. 24. The signature genes induced by the chemical constituent Albiflorin in a complex herbal preparation, Huang Chin Tang, in Jurkat T cell. (A) show the genes that were positively correlated with Albiflorin, and (B) shows the genes that were negatively correlated with Albiflorin.

FIG. 25. Correlation of gene expression profiles to a control group. (A) is the gene expression profile of a control group, and (B) is the gene expression profile of a sample group. (C) shows the number of genes with a differential expression ratio having greater than 2-fold increase with concentration of herbal treatment.

FIG. 26. Clusters of expression profiles clustered by a non-hierarchical analysis program, wherein the program is based on a self-organizing map (SOM) principle. The X-axis represents the herbal concentration from low to high and the Y-axis is the gene-expression ratio.

FIG. 27. FIG. 27 shows the induced and repressed genes commonly found in two batches of Huang Chin Tang.

FIG. 28. SOM clustering results for two batches of Huang Chin Tang. (A) shows the SOM clustering results for the expression profiles of two batches of Huang Chin Tang. (B) shows that ten genes have similarly responded to the two batches, and (C) shows how the weighing factor decreases as cluster I and cluster j become more different.

FIG. 29. Calculation of S score between pairs of herbal preparations in cluster analysis. (A) is a tabulation of the scores, and (B) is demonstrates how 5 batches of similar herbal preparations are related.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Overview of the Invention

As set forth above, the present invention is directed to tools and methods useful for predicting the biological response of an herbal composition. More particularly, this invention provides methods of creating Herbal BioResponse Array (HBR Array) databases as well as methods for using such databases to improve the design of effective herbal-based therapeutics. The goal of the present invention is the overall design, creation, improvement and use of HBR Arrays for the preparation, testing and administration of herbal compositions, and guide development of new herbal compositions and novel uses of existing herbal compositions.

Phytomics. As used herein, depending on the context in which it is used, “phytomics” refers to using bioinformatics and statistical approaches to address the qualitative and quantitative aspects of the components of herbal compositions or to the actual data bases which are developed for addressing such aspects.

Herbal BioResponse Array. As used herein, an HBR Array constitutes a data set of two or more observations or measurements associated with an herbal composition. The HBR Array may include qualitative and quantitative data on the plants in the composition (plant-related data), marker information obtained after exposure of a biosystem to the herbal composition including a dose dependent study, and a database of BioResponse data obtained after exposure of a biosystem to the herbal composition. The data in any particular HBR Array can be statistically analyzed in either 2- or 3-dimensional space.

HBR Arrays may be designated as batch HBR Arrays and standardized HBR Arrays. Batch HBR Arrays are arrays of data associated with specific batches of an herbal composition. Standardized HBR Arrays are arrays of data associated with a standardized herbal composition.

Major Data Set. As used herein, the term “major data set” refers to the data set which acts as the baseline set of data by which various other sets of data are compared or otherwise analyzed for the same or different herbal compositions. Generally, the major data set is created using biotechnological techniques to ascertain some genetic or protein aspect of the herbal compositions. Thus, the major data set will usually, but not always, be based on a genomic or proteomic set of data. For example, nucleic acid microarray results could be the major data set which is used to compare to other, dependent or minor data sets.

Minor or Dependent Data Set. As used herein, the “minor data set” or “dependent data set” refers to one or more data sets which are used for comparing to the major data set. Generally, but not always, them minor data set will consist of information on an herbal composition which are collected by more traditional methods. For example, the minor, or dependent, data set may consist of a collection of plant-related data obtained by more conventional means. Examples of plant-related data include, but are not limited to, the genus/species of the herb(s) in the herbal composition, the particular plant parts of the herb(s) in the composition and the geographic location where the herb(s) were located. Another example of a minor data set might consist of a set of biological responses of a cell, tissue, organ or organism after treatment with one or more different amounts of the herbal composition. Examples of such biological data or a whole organism may include, but are not limited to, cell toxicity studies, enzyme treatment studies, growth rates, weight gain or loss, changes in motor skills and changes in mental abilities.

Herb. Technically speaking an herb is a small, non-woody (i.e., fleshy stemmed), annual or perennial seed-bearing plant in which all the aerial parts die back at the end of each growing season. Herbs are valued for their medicinal, savory or aromatic qualities. As the word is more generally used and as the word is used herein, an “herb” refers to any plant or plant part which has a food supplement, medicinal, drug, therapeutic or life-enhancing use. Thus, as used herein, an herb is not limited to the botanical definition of an herb but rather to any botanical, plant or plant part used for such purposes, including any plant or plant part of any plant species or subspecies of the Metaphyta kingdom, including herbs, shrubs, subshrubs, and trees. Plant parts used in herbal compositions include, but are not limited to, seeds, leaves, stems, twigs, branches, buds, flowers, bulbs, corms, tubers, rhizomes, runners, roots, fruits, cones, berries, cambium and bark.

Herbal Composition. As used herein, an “herbal composition” refers to any composition which includes herbs, herbal plants or herbal plant parts. Thus, as used herein, an herbal composition is any herbal preparation, including herbal food supplements, herbal medicines, herbal drugs and medical foods. Examples of herbal compositions include, but are not limited to, the following components: a whole plant or a plant part of a single plant species; whole plants or plant parts of multiple plant species; multiple components derived from a single plant species; multiple components derived from multiple plant species; or any combination of these various components. For a thorough review of various herbal compositions, see, for example, Kee Chang Huang, The Pharmacology of Chinese Herbs, CRC Press (1993), herein incorporated in its entirety. Representative examples of various herbal compositions are provided in the following paragraphs.

Herbal compositions which include the bark of the willow tree have been used to treat fever since the mid-eighteenth century in England. The active ingredient in willow bark is a bitter glycoside called salicin, which on hydrolysis yields glucose and salicylic alcohol. Aspirin (acetylsalicylic acid) and aspirin-like drugs (e.g., ibuprofen), all of which are often called nonsteroidal antiinflammatory drugs (NSAIDs), are frequently used to treat pain, fever, and inflammation. Meadowsweet is another herb that contains salicylates. Treatment of arthritic and arthritic-like symptoms with willow bark or meadowsweet requires the consumption of large quantities of herbal teas made from these plants. The entire Populus species (i.e., poplar trees and shrubs) also contains salicylate precursors and poplar-buds have been used in antiinflammatory antipyretic and analgesic medications.

U.S. patents have been issued for herbal compositions used for the treatment of various diseases and other health-related problems afflicting humans and animals. For example, U.S. Pat. No. 5,417,979 discloses a composition comprising a mixture of herbs, including species of Stephania and Glycyrrhiza; as well as their extracts, which is used as an appetite stimulant and for the treatment of pain. Herbal compositions which include Glycyrrhiza uralensis have been found useful for treating eczema, psoriasis, pruritis and inflammatory reactions of the skin (U.S. Pat. No. 5,466,452). U.S. Pat. No. 5,595,743 discloses various herbal compositions which include licorice extract (Glycyrrhiza) and siegesbeckia, sophora, stemona and tetrandra herbs used for the treatment of various mammalian diseases, including inflammation and rheumatoid arthritis. Ocular inflammation can be treated with a pharmaceutical composition containing the plant alkaloid tetrandrine (U.S. Pat. No. 5,627,195).

U.S. Pat. No. 5,683,697 discloses a pharmaceutical composition having anti-inflammatory, anti-fever, expectorant or anti-tussive action, wherein the composition includes plant parts from the species Melia, Angepica, Dendrobium, Impatiens, Citrus, Loranthus, Celosia, Cynanchum and Glehnia. An herbal composition which includes extracts of the roots, rhizomes, and/or vegetation of Alphinia, Smilax, Tinospora, Tribulus, Withania and Zingiber has been found to reduce or alleviate the symptoms associated with rheumatoid arthritis, osteoarthritis, reactive arthritis and for reducing the production of proinflammatory cytokines (U.S. Pat. No. 5,683,698).

Herbal compositions are available in many forms, including capsules, tablets, or coated tablets; pellets; extracts or tinctures powders; fresh or dried plants or plant parts; prepared teas; juices; creams and ointments; essential oils; or, as combinations of any of these forms. Herbal medicines are administered by any one of various methods, including orally, rectally, parenterally, enterally, transdermally, intravenously, via feeding tubes, and topically.

Herbal compositions encompassed by the present invention include herbal compositions which also contain non-herbal components. Examples of such non-herbal components include, but are not limited to, whole insects and insect parts, worms, animal or insect feces, natural or petroleum boils, carbonate of ammonia, salt of tartar, liquor, water, glycerin, steroids, pharmaceuticals, vitamins, nutrient extracts, whey, salts, and gelatin.

For oral administration, the herbal compositions disclosed may take the form of, for example, tablets or capsules prepared by conventional means in admixture with generally acceptable excipients such as binding agents (e.g., pregelatinised maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate); glidants; artificial and natural flavors and sweeteners; artificial or natural colors and dyes; and solubilizers. The herbal compositions may be additionally formulated to release the active agents in a time-release manner as is known in the art and as discussed in U.S. Pat. Nos. 4,690,825 and 5,055,300. The tablets may be coated by methods well known in the art.

Liquid preparations for oral administration may take the form of, for example, solutions, syrups, suspensions, or slurries (such as the liquid nutritional supplements described in Mulchandani et al., 1992 U.S. Pat. No. 5,108,767), or they may be presented as a dry product for reconstitution with water or other suitable vehicles before use. Liquid preparations of folic acid, and other vitamins and minerals may come in the form of a liquid nutritional supplement specifically designed for ESRD patients. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

For topical administration, herbal components may be combined in admixture with at least one other ingredient constituting an acceptable carrier, diluent or excipient in order to provide a composition, such as a cream, gel, solid, paste, salve, powder, lotion, liquid, aerosol treatment, or the like, which is most suitable for topical application. Sterile distilled water alone and simple cream, ointment and gel bases may be employed as carriers of the herbal components. Preservatives and buffers may also be added. The formulation may be applied to a sterile dressing, biodegradable, absorbable patches or dressings for topical application, or to slow release implant systems with a high initial release decaying to slow release.

For a more complete overview and discussion of herbal-based compositions see Earl Mindell, Earl Mindell's Herb Bible. Simon & Schuster (1992); Culpeper's Complete Herbal, W. Foulsham & Co., Ltd. (orginally published in the mid 1600's); and, Rodale's Illustrated Encyclopedia of Herbs, Rodale:Press (1987).

Standardized Herbal Composition. As used herein, a “standardized herbal composition” or a “characterized herbal composition” refers to a particular herbal composition which is chosen as the standard herbal composition for evaluating batch herbal compositions which have the same, similar or different components as the components of the standardized herbal composition. Sometimes herein also referred to as the “master herbal composition.” Standardized herbal compositions are generally herbal compositions which have been well characterized and which demonstrate the desired biological responses in a particular biosystem. Standardized herbal compositions are usually standardized by chemical tests well known to one skilled in the art and are properly stored for long term usage and reference. The standardized herbal composition is used to establish a standardized HBR Array based on observations and measurements for the plants (ie., plant-related data), markers and BioResponses so as to characterize the herbal composition.

Batch Herbal Composition. As used herein, a “batch herbal composition” refers to any test herbal composition which is used to establish a HBR Array based on observations and measurements for the plants and markers so as to characterize the herbal composition. Sometimes herein also referred to as a “test” or “batch” herbal composition. Observations and measurements of BioResponses may or may not be included. The herbal compositions used to establish the standardized herbal composition may also be referred to as “batch herbal compositions” until designated as “standardized herbal compositions.”

Batch. As used herein, a “batch” refers to a particular quantity of an herbal composition which can be identified as to some particular attribute so as to distinguish it from any other particular quantity of that same herbal composition. For example, one batch of an herbal composition may differ from another batch of that same herbal composition in that one of the batches was harvested at a different time or in a different geographical location than the other batch. Other differences that distinguish particular batches may include, but are not limited to, the following; 1) the particular plant part used (e.g., the root of an herb was used in one batch while the leaves of that same herb were used in a different batch); 2) the post-harvest treatment of the individual herbs or herbal composition (e.g., one batch may be processed with distilled water while a different batch may be processed with Hydrogen Chloride to simulate the acidity of the human stomach); and, 3) the relative proportions of the individual herbs in an herbal composition (e.g., one batch may have equal parts by weight or volume of three different herbs while another batch has proportionally more of one herb than the other two).

Biosystem. As used herein, a “biosystem” refers to any biological entity for which biological responses may be observed or measured. Thus, a biosystem includes, but is not limited to, any cell, tissue, organ, whole organism or in vitro assay.

Biological Activity. As used herein, the “biological activity” of an herb refers to the specific biological effect peculiar to an herbal composition on a given biosystem.

Plant-Related Data. As used herein, “Plant-related data” refers to the data collected on the herbal composition including, but not limited to, data about the plants, their growing conditions and the handling of the plants during and after harvesting. The plant-related data also includes the relative proportions of the components in an herbal compositions, wherein the components may be different plant parts, different plant species, other non-plant ingredients (e.g., insect parts, chemical drugs) or any combinations of these variables.

Plant-related data which may be gathered for an herbal composition includes, but is not limited to, the following: 1) the plant species (and, if available, the specific plant variety, cultivar, clone, line, etc.) and specific plant parts used in the composition; 2) the geographic origin of the herbs, including the longitude/latitude and elevation; 3) the growth conditions of the herbs, including fertilizer types and amounts, amounts and times of rainfall and irrigation, average microEinsteins received per day, pesticide usage, including herbicides, insecticides, miticides and fungicides, and tillage methods; 4) methods and conditions used for processing the herbs, including age/maturity of the herbs, soaking times, drying times, extraction methods and grinding methods; and 5), storing methods and conditions for the herbal components and the final herbal composition.

Additionally, the standardized herbal composition may be analyzed chemically. Chemical characterization may be accomplished by any chemical analysis method generally known by one skilled in the art. Examples of applicable chemical analyses include, but are not limited to, HPLC, TLC, chemical fingerprinting, mass spectrophotometer analyses and gas chromatography.

Cell Banking System. As used herein, a “cell banking system” includes a Master Cell Bank (MCB) and a Working Cell Bank (WCB) of cells. The use of a cell banking system minimizes cell variability for herbal medicine testing, and is used for all types of cells in nucleic acid microarray studies.

Bioinformatics. As used herein, “bioinformatics” refers to the use and organization of information of biological interest. Bioinformatics covers, among other things, the following: (1) data acquisition and analysis; (2) database development; (3) integration and links; and (4) further analysis of the resulting database. Nearly all bioinformatics resources were developed as public domain freeware until the early 1990s, and much is still available free over the Internet. Some companies have developed proprietary databases or analytical software.

Genomic or Genomics. As-used herein, the term “genomics” refers to the study of genes and their function. Genomics emphasizes the integration of basic and applied research in comparative gene mapping, molecular cloning, large-scale restriction mapping, and DNA sequencing and computational analysis. Genetic information is extracted using fundamental techniques, such as DNA sequencing, protein sequencing and PCR.

Gene function is determined (1) by analyzing the effects of DNA mutations in genes on normal development and health of the cell, tissue, organ or organism; (2) by analyzing a variety of signals encoded in the DNA sequence; and (3) by studying the proteins produced by a gene or system of related genes.

Proteomic or Proteomics. As used herein, the term “proteomics”, also called “proteome research” or “phenome”, refers to the quantitative protein expression pattern of a genome under defined conditions. As used generally, proteomics refers to methods of high throughput, automated analysis using protein biochemistry.

Conducting proteome research in addition to genome research is necessary for a number of reasons. First, the level of gene expression does not necessarily represent the amount of active protein in a cell. Also, the gene sequence does not describe post-tranlsational modifications which are essential for the function and activity of a protein. In addition, the genome itself does not describe the dynamic cell processes which alter the protein level either up or down.

Proteome programs seek to characterize all the proteins in a cell, identifying at least part of their amino acid sequence of an isolated protein. In general, the proteins are first separated using 2D gels or HPLC and then the peptides or proteins are sequenced using high throughput mass spectrometry. Using a computer, the output of the mass spectrometry can be analyzed so as to link a gene and the particular protein for which it codes. This overall process is sometimes referred to as “functional genomics”. A number of commercial ventures now offer proteomic services (e.g., Pharmaceutical Proteomics™, The ProteinChip™ System from Ciphergen Biosystem, PerSeptive Biosystems).

For general information on proteome research, see, for example, J. S. Fruton, 1999, Proteins, Enzymes, Genes: The Interplay of Chemistry and Biology, Yale Univ. Pr.; Wilkins et al., 1997, Proteome Research: New Frontiers in Functional Genomics (Principles and Practice), Springer Verlag; A. J. Link; 1999, 2-D Proteome Analysis Protocals (Methods in Molecular Biology, 112, Humana Pr. Kamp et al., 1999, Proteome and Protein Analysis, Springer Verlag.

Signal Transduction. As used herein, “signal transduction”, also known as cellular signal transduction refers to the pathways through which cells receive external signals and transmit, amplify and direct them internally. Signaling pathways require intercommunicating chains of proteins that transmit the signal in a stepwise fashion. Protein kinases often participate in this cascade, of reactions, since many signal transductions involve receiving an extracellular chemical signal, which triggers the phosphorylation of cytoplasmic proteins to amplify the signal.

Post-translational-Modification. As used herein, “post-translational modification” is a blanket term used to cover the alterations that happen to a protein after it has been synthesized as a primary polypeptide: Such post-translational modifications include, but are not limited to, glycosylation, removal of the N-terminal methionine (or N-formyl methionine), signal peptide removal, acetylation, formylation, amino acid modifications, internal cleavage of peptide chains to release smaller proteins or peptides, phosphorylation, and modification of methionine.

Array or Microarray. As used herein, an “array” or “microarray” refers to a grid system which has each position or probe cell occupied by a defined nucleic acid fragment. The arrays themselves are sometimes referred to as “chips”, “biochips”, “DNA chips” or “gene chips”. High-density nucleic acid microarrays often have thousands of probe cells in a variety of grid styles.

Once the array is fabricated, DNA or protein molecules derived from a biosystem are added and some form of chemistry occurs between the DNA or protein molecules and the array to give some recognition pattern that is particular to that array and biosystem. Autoradiography of radiolabeled batches is a traditional detection strategy, but other options are available, including fluorescence, colorimetry, and electronic signal transduction.

Markers. As used herein, the term “markers” refers to any biological-based measurement or observation for a particular herbal composition that is characteristic of a particular biosystem which is being exposed to a particular batch of an herbal composition. The term “marker” encompasses both qualitative and qualitative measurements and observations of a biosystem. The Marker database constitutes a data set which characterizes gene expression patterns in response to herbal therapies, wherein the patterns show which genes are turned on, off, up, or down in response to specific herbal compositions. Thus, “markers” refers to any biologically based measurement or observation whose up- and down- or temporal regulations, or qualitative or quantitative changes of expression levels in a biosystem are used to characterize differential biological responses of a biosystem to an herbal composition.

The particular batch of an herbal composition to which the biosystem is exposed may be an unknown herbal composition, a known herbal composition, or a standardized herbal composition. Examples of markers useful in accomplishing the present invention include, but are not limited to, molecular markers, cytogenetic markers, biochemical markers or macromolecular markers. Macromolecular markers include, but are not limited to, enzymes, polypeptides, peptides, sugars, antibodies, DNA, RNA, proteins (both translational proteins and post-translational proteins), nucleic acids, polysaccharides.

Any marker that satisfies the definition of “marker” herein is appropriate for conducting the present invention. The term “markers” includes related, alternative terms, such as “biomarker” or “genetic marker” or “gene marker.” There may be one or more primary markers along with secondary markers, or a hierarchy of markers for achieving the purposes of increasing the discriminating power of a HBR array. Thus, selected molecular markers may be combined with various other molecular, cytogenetic, biochemical or macromolecular markers to enable an even more accurate, extended HBR Array.

A molecular marker comprises one or more microscopic molecules from one or more classes of molecular compounds, such as DNA, RNA, cDNA, nucleic acid fragments, proteins, protein fragments, lipids, fatty acids, carbohydrates, and glycoproteins.

The establishment, generation and use of applicable molecular markers are well known to one skilled in the art. Examples of particularly useful technologies for the characterization of molecular markers include differential display, reverse transcriptase polymerase chain reactions (RT-PCR), large-scale sequencing of expressed sequence tags (ESTs), serial analysis of gene expression (SAGE), Western immunoblot or 2D, 3D study of proteins, and microarray technology. One skilled in the art of molecular marker technology is familiar with the methods and uses of such technology (see, e.g., Bernard R. Glick and Jack J. Pasternak, Molecular Biotechnology, Principles and Applications of Recombinant DNA, Second Edition, ASM Press (1998); Mathew R. Walker and Ralph Rapley, Route Maps in Gene Technology, Blackwell Science (1997); Roe et al., DNA Isolation and Sequencing, John Wiley & Sons (1996)James D. Watson et al., Recombinant DNA. Second Edition, Scientific American Books (1992)).

DNA, RNA and protein isolation and sequencing methods are well known to those skilled in the art. Examples of such well known techniques can be found in Molecular Cloning: A Laboratory Manual 2nd Edition, Sambrook et al., Cold Spring Harbor, N.Y. (1989); Hanspeter Saluz and J. P. Jost, A Laboratory Guide to Genomic Sequencing: The Direct Sequencing of Native Uncloned DNA (Biomethods Vol 1), Birkhauser (1988); and B. Roe et al., DNA Isolation and Sequencing, Wiley (1996). Examples of conventional molecular biology techniques include, but are not limited to, in vitro ligation, restriction endonuclease digestion, PCR, cellular transformation, hybridization, electrophoresis, DNA sequencing, cell culture, and the like. Specific kits and tools available commercially for use in the present invention include, but are not limited to, those useful for RNA isolation, PCR cDNA library construction, retroviral expression libraries, vectors, gene expression analyses, protein antibody purification, cytotoxicity assays, protein expression and purification, and high-throughput plasmid purification (see, e.g., CLONTECHniques product catalog, XIII(3), 1-32 (1998) or www.clontech.com; Atlas™ cDNA Expression Assays product catalog (1998); SIGMA® product catalog (1997)).

For discussions, methodologies and applications of oligonucleotide arrays, microarrays, DNA chips or biochips, ;see, for example, U.S. Pat. Numbers 5,445,934, 5,605,662, 5,631,134, 5,736,257, 5,741,644, 5,744,305, 5,795,714; Schena et al., Parallel human genome analysis: Microarray-based expression monitoring of 1000 genes, Proc. Natl. Acad. Sci. USA 93, 10614-10619 (1996); DeRisi et al., Exploring the Metabolic and Genetic Control of Gene Expression on a Genomic Scale, Science 278, 680-686 (1997); Wodicka, et al., Genome-wide Expression Monitoring in Saccharomyces cerevisiae, Nature Biotechnology 15, 1359-1367 (1997); Pardee, Complete Genome Expression Monitoring: The Human Race, Nature Biotechnology 15, 1343-1344,(1997); Schafer et al., DNA Variation and the Future of Human Genetics, Nature Biotechnology 16, 33-39 (1998); DeRisi et al., Use of a cDNA Microarray to Analyze Gene Expression Patterns in Human Cancer, Nature Genetics 14, 457-460 (1996); Heller et al., Discovery and Analysis of Inflammatory Disease-Related Genes Using cDNA Microarrays, Proc. Natl. Acad. Sci. USA 94, 2150-2155 (1997); Marshall et al., DNA Chips: An Array of Possibilities, Nature Biotechnology 16, 27-31 (1998); Schena et al., Microarrays: Biotechnology's Discovery Platform for Functional Genomics, Tibtech 16, 301-306 (1998); Ramsay, DNA Chips: State-of-the-art, Nature Biotechnology 16, 40-44 (1998); Chee et al., Accessing Genetic Information wit High-Density DNA Arrays, Science 274, 610-614 (1996); and Chen et al., Profiling Expression Patterns and Isolating Differentially Expressed Genes by cDNA Microarray System with Colorimetry Detection, Genomics 50, 1-12 (1998); P. Andrew Outinen et al., Characterization of the stress-inducing effects of homocysteine, Biochem. J. 332, 213-221 (1998); and Gelbert et al., Will genetics really revolutionize the drug discovery process, Curr Opin Biotechol 8(6), 669-674-(1997).

Other, more specific, references applicable to the instant invention include, but are not limited to, those addressing the expression technologies, such as ESTs (see, e.g., Michael R. Fannon, Gene expression in normal and disease states—identification of therapeutic targets, TIBTECH 14, 294-298 (1996)); the generation of protein profiles (see, e.g., Robinson et al., A Tyrosine Kinase Profile of Prostate Carcinoma, Proc. Natl. Acad. Sci. USA 93, 5958-5962 (1996)); chemical and spectroscopic methods for identifying components of herbal compositions (Kojima et al., Saponins from Gliricidia sepium, Phytochemistry 48(5), 885-888 (1998)); the determination of functional antigens (see, e.g., Aris Persidis, Functional antigenics, Nature Biotechnology 16, 305-307 (1998)); HPLCs (see, e.g., Milton T. W. Hearn (Editor), HPLC of Proteins Pepties, and Polynucleotides: Contemporary Topics and Applications (Analytical Techniques in Clinical Chemistry and Laboratory Manual), VCH Pub. (1991); electrophoresis (see, e.g., Westermeier et al., Electrophoresis in Practice: A Guide to Methods and Applications of DNA and Protein Separations, John Wiley & Sons (1997)); and cross-reactivity marker assays (see, e.g., Irving Millman et al., Woodchuck Hepatitis Virus: Experimental Infection and Natural Occurrence, Hepatology 4(5):817-823 (1984)). The use of structural genomics for solving the structures of all the proteins encoded for in completed genomes, wherein the methodology includes high-throughput direct structure determinations and computational methods, is discussed by Terry Gaasterland, Structural genomics: Bioinformatics in the driver's seat, Nature Biotechnology 16, 625-627. For bioinformatics methodologies, see, for example, Andreas Baxevanis (Editor), Bioinformatics: A Practical Guide to the Analysis of Genes and Proteins, John Wiley & Sons (1998) and Luke Alphey, DNA Sequencing: From Experimental Methods to Bioinformatics (Introduction to Biotechniques Series), Springer Verlag (1997).

Cytogenetic parameters include, but are not limited to, karyotype analyses (e.g., relative chromosome lengths, centromere positions, presence or absence of secondary constrictions), ideograms (i.e., a diagrammatic representation of the karyotype of an organism), the behavior of chromosomes during mitosis and meiosis, chromosome staining and banding patterns, DNA-protein interactions (also known as nuclease protection assays), neutron scattering studies, rolling circles (A. M. Diegehlan and E. T. Kool, Nucleic Acids Res 26(13):3235-3241 (1998); Backert et al., Mol. Cell. Biol. 16(11):6285-6294 (1996); Skaliter et al., J. Viol. 70(2):1132-1136 (1996) A. Fire and S. Q. Xu, Proc. Natl. Acad. Sci. USA 92(10):4641-4645 (1995)), and autoradiography of whole nuclei following incubation with radiolabelled ribonucleotides.

Biochemical parameters include, but are not limited to, specific pathway analyses, such as signal transduction, protein synthesis and transport, RNA transcription, cholesterol synthesis and degradation, glucogenesis and glycolysis.

Fingerprinting. As used herein, the term “fingerprinting” as used herein refers to the means of making a characteristic profile of a substance, particularly an herb, in order to identify it. The term “fingerprint” as used herein refers to the display of the result of the particular means employed for the fingerprinting.

Examples of the various types of fingerprinting means include, but are not limited to, DNA fingerprinting, protein finger printing, chemical fingerprinting and footprinting.

DNA fingerprinting, or profiling, refers to a way of making a unique pattern from the DNA of particular biological source (e.g., a particular plant, plant species, genus of plant, plant part or plant tissue). The DNA fingerprint, or profile, can be used to distinguish that particular biological source from a different biological source. The patterns obtained by analyzing a batch using microarrays, oligonucletide arrays, DNA chips or biochips are also referred to as “fingerprints”.

Protein fingerprinting refers to generating a pattern of proteins in a cell, tissue, organ or organism, such as a plant, which provides a completely characteristic “fingerprint” of that cell, tissue, organ or organism at that time.

Chemical fingerprinting refers to the analysis of the low molecular weight chemicals in a cell and the resulting pattern used to identify a cell, tissue, organ or organism, such as a plant. The analysis is usually done using Gas Chromatography (GC), HPLC or mass spectrometry.

Footprinting refers to a method of finding how two molecules stick together. In the case of DNA, a protein is bound to a labeled piece of DNA, and then the DNA is broken down, by enzymes or by chemical attack. This process produces a “ladder” of fragments of all sizes. Where the DNA is protected by the bound protein it is degraded less, and so the “ladder” appears fainter. Footprinting is a common technique for homing in on where the proteins that regulate gene activity actually bind to the DNA.

The means, or methods, used to accomplish each type of fingerprinting are described in detail elsewhere herein.

BioResponses. As used herein, a “BioResponse” refers to any observation or measurement of a biological response of a biosystem following exposure to an herbal composition. Sometimes herein also referred to as a “biological effect.” A BioResponse is a qualitative or quantitative data point for the biological activity of a particular herbal composition. BioResponse data includes both dosage and temporal information, wherein such information is well known to one skilled in the art of measuring responses of biosystems to various treatments. Thus, BioResponse data includes information on the specific biological response of a specific biosystem to a specific dosage of herbal composition administered in a particular manner for a specific period of time.

BioResponses include, but are not limited to, physiological responses, morphological responses, cognitive responses, motivational responses, autonomic responses and post-transitional modification's, such as signal transduction measurements. Many herbal compositions demonstrate more than one BioResponse (see, e.g., Kee Chang Huang, The Pharmacology of Chinese Herbs, CRC Press (1993)). Some particular BioResponses may be included in more than one of the delineated groups or have aspects or components of the response that encompass more than one group. BioResponses applicable to the instant invention are well known to one skilled in the art. The following references are representative of the state of art in the field: Kee Chang Huang, The Pharmacology of Chinese Herbs, CRC Press (1993); Earl Mindell, Earl Mindell's Herb Bible, Simon & Schuster (1992); Goodman & Gilman's The Pharmacological Basis of Therapeutics, Ninth Edition, Joel G. Hardman, et. al. (eds.), McGraw Hill, Health Professions Division (1996); P. J. Bentley, Elements of pharmacology. A primer on drug action, Cambridge University Press (1981); P. T. Marshall and G. M. Hughes, Physiology of mammals and other vertebrates. Second Edition, Cambridge University Press (1980); Report of the Committee on Infectious Diseases, American Academy of Pediatrics (1991); Knut Schmidt-Nielsen, Animal Physiology: Adaptation and Environment, 5th Edition, Cambridge University Press (1997); Elain N. Marieb, Human Anatomy & Physiology, Addison-Wessey Pub. Co. (1997); William P. Ganong, Review of Medical Physiology (18th Ed), Appleton & Lange (1997); Arthur C. Guyton and John E. Hall, Textbook of Medical Physiology, W. B. Saunders Co. (1995).

A “physiological response” refers to any characteristic related to the physiology, or functioning, of a biosystem. Physiological responses on a cellular, tissue or organ level include, but are not limited to, temperature, blood flow rate, pulse rate, oxygen concentration, bioelectric potential, pH value, cholesterol levels, infection state (e.g., viral, bacterial) and ion flux. Physiological responses on a whole organism basis include gastrointestinal functioning (e.g., ulcers, upset stomach, indigestion, heartburn), reproductive tract functioning (e.g., physiologically-based impotence, uterine cramping, menstrual cramps), excretory functions (e.g., urinary tract problems, kidney ailments, diarrhea, constipation), blood circulation (e.g., hypertension, heart disorders), oxygen consumption, skeletal health (e.g., osteoporosis), condition of the cartilage and connective tissues (e.g., joint pain and inflammation), locomotion, eyesight (e.g., myopia, blindness), muscle tone (e.g. wasting syndrome, muscle strains), presence or absence of pain, epidermal and dermal health (e.g., skin irritation, itching, skin wounds), functioning of the endocrine system, cardiac functioning, nervous coordination, head-related health (e.g., headaches, dizziness), age (e.g., life span, longevity) and respiration (e.g., congestion, respiratory ailments).

A “morphological response” refers to any characteristic related to the morphology, or the form and structure, of a biosystem following exposure to an herbal composition. Morphological responses, regardless of the type of biosystem, include, but are not limited to, size, weight, height, width, color, degree of inflammation, general appearance (e.g., opaqueness, transparency, paleness), degree of wetness or dryness, presence or absence of cancerous growths, and the presence or lack of parasites or pests (e.g., mice, lice, fleas). Morphological responses on a whole organism basis include, but are not limited to, the amount and location of hair growth (e.g., hirsutism, baldness), presence or absence of wrinkles, type and degree of nail and skin growth, degree of blot clotting, presence or absence of sores or wounds, and presence or absence of hemorrhoids.

A “cognitive response” refers to any characteristic related to the cognitive, or mental state, of a biosystem following exposure to an herbal composition. Cognitive responses include, but are not limited to, perceiving, recognizing, conceiving, judging, memory, reasoning and imagining.

A “motivational response” refers to any characteristic related to the motivation, or induces action, of a biosystem following exposure to an herbal composition. Motivational responses include, but are not limited to, emotion (e.g., cheerfulness), desire, learned drive, particular physiological needs (e.g., appetite, sexual drive) or similar impulses that act as incitements to action (e.g., stamina, sex drive).

An “autonomic response” refers to any characteristic related to autonomic responses of a biosystem following exposure to an herbal composition. Autonomic responses are related to the autonomic nervous system of the biosystem. Examples of autonomic responses include, but art not limited to, involuntary functioning (e.g., nervousness, panic attacks), or physiological needs (e.g., respiration, cardiac rhythm, hormone release, immune responses, insomnia, narcolepsy).

BioResponses of cells, tissues, organs and whole organisms treated with various herbal compositions or herbal components are well known in the herbal arts. For example, the herbal compositions Sairei-to (TJ-114), alismatis rhizoma (Japanese name ‘Takusha’) and hoelen (Japanese name ‘Bukuryou’) were each found to inhibit the synthesis and expression of endothelin-1 in rats (Hattori et al., Sairei-to may inhibit the synthesis of endothelin-1 in nephritic glomeruli, Nippon Jinzo Gakkai Shi 39(2), 121-128 (1997)). Interleukin (L)-1 alpha production was significantly promoted by treatment of cultured human epidermal keratinocytes with the herbal medicine Sho-saiko-to (Matsumoto et al., Enhancement of interleukin-1 alpha mediated autocrine growth of cultured human keratinocytes by sho-saiko-to, Jpn J. Pharmacol 73(4), 333-336 (1997). Adding Sho-saiko-to to a culture of peripheral blood mononuclear cells obtained from healthy volunteers resulted in a dose-dependent increase in the production of granulocyte colony-stimulating factor (G-CSF) (Yamashiki et al., Herbal medicine “sho-saiko-to” induces in vitro granulocyte colony-stimulating factor production on peripheral blood mononuclear cells, J Clin Lab Immunol 37(2), 83-90 (1992)). These researchers concluded that the administration of Sho-saiko-to may be useful for the treatment of chronic liver disease, malignant diseases and acute infectious diseases where G-CSF is efficacious. Plasminogen activator inhibitor type 1 .(PAI-1)-specific mRNA expression decreased and tissue-type plasminogen activator (t-PA)-specific mRNA increased after treatment of human umbilical vein endothelial cells (HUVECs) with the saponin astragaloside IV (AS-IV) purified from the Chinese herb Astragalus membranaceus (Zhang et al., Regulation of the fibrinolytic potential of cultured human umbilical vein endothelial cells: astragaloside IV down regulates plasminogen activator inhibitor-1 and up regulates tissue-type plasminogen activator expression, J Vasc Res 34(4),273-280(1997)). One component out of four components isolated from the roots of Panax ginseng was found to be a potent inducer of IL-8 production by human monocytes and THP-1 cells, and this induction was accompanied by increased IL-8 mRNA expression (Sonoda et al., Stimulation of interleukin-8 production by acidic polysaccharides from the root of panax ginseng, Immunopharmacology 38(3), 287-294 (1998)). By flow cytometric analysis, the expression of Fc gamma 11/111 receptors and complement receptor 3(CR3) on macrophages were found to be increased by treatment with the Kampo-herbal medicine Toki-shakuyakusan (TSS) (Cyong, New BRM from kampo-herbal medicine, Nippon Yakurigaku Zasshi 110 Suppl 1, 87P-92P (1997)). Using computer image analysis, Chen et al. (Image analysis for intercellular adhesion molecule-1 expression in MRI/1pr mice: effects of Chinese herb medicine, Chung Hua I Hsueh Tsa Chih 75(4), 204-206 (1995)) found that the distribution intensity of intercellular adhesion molecule-1 (ICAM-1), immunoglobulins and C3 were significantly decreased in MRL/1pr mice after treatment with the Chinese herb stragalin. Western blot analysis showed that tetradrine, isolated from a natural Chinese herbal medicine, inhibited signal-induced NF-kappa B activation in rat alveolar macrophages (Chen et al., Tetrandrine inhibits signal-induced NF-kappa B activation in rat alveolar macrophages, Biochem Biophys Res Commun 231(1), 99-102 (1997)).

Algorithm. As used herein, an “algorithm” refers to a step-by-step problem-solving procedure, especially an established recursive computational procedure with a finite number of steps. Appropriate algorithms for two- and three-dimensional analyses of the plant-related, marker and BioResponse data sets are well known to one skilled in the computational arts. Such algorithms are useful in constructing the Herbal BioResponse Arrays of the present invention. For general information on algorithms, see, for example, Jerrod H. Zar, Biostatistical Analysis, second edition, Prentice Hall (1984); Robert A. Schowengerdt, Techniques for image processing and classification in remote sensing, Academic Press (1983); Steven Gold et al., New Algorithms for 2D and 3D Point Matching: Pose Estimation and Correspondence, Pattern Recognition, 31(8):1019-1031 (1998); Berc Rustem, Algorithms for Nonlinear Programming and Multiple-Objective Decisions, Wiley-Interscience Series in Systems and Optimization, John Wiley & Sons (1998); Jeffrey H. Kingston, Algorithms and Data Structures: Design, Correctness, Analysis, International Computer Science Series, Addison-Wesley Pub. Co (1997); Steven S. Skiena, The Algorithm Design Manual, Springer Verlag (1997); and Marcel F. Neuts, Algorithm Probability: A Collection of Problems (Stochastic Modeling), Chapman & Hall (1995). For information more specific to the application of algorithms to genetic-based data, see, for example, Dan Gusfield, Algorithms on Strings, Trees, and Sequences: Computer Science and Computational Biology, Cambridge University Press (1997); Melanie Mitchell, An Introduction to Genetic Algorithms (Complex Adaptive Systems), MIT Press (1996); David E. Goldberg, Genetic Algorithms in Search, Optimization and Machine Learning, Addison-Wessley Pub. Co. (1989); Zbigniew Michalewicz, Genetic Algorithms+Data Structures=Evolution Programs, Springer Verlag (1996); Andre G. Uitterlinden and Jan Vijg, Two-Dimensional DNA Typing: A Parallel Approach to Genome Analysis, Ellis Horwood Series in Molecular Biology, Ellis Horwood Ltd. (1994); and Pierre Baldi and Soren Brunak, Bioinformatics: The Machine Learning Approach (Adaptive Computation and Machine Learning), MIT Press (1998).

Combinatorial Chemistry. As used herein, “combinatorial chemistry” refers to the numerous technologies used to, create hundreds or thousands of chemical compounds, wherein each of the chemical compounds differ for one or more features, such as their shape, charge, and/or hydrophobic characteristics. Combinatorial chemistry can be utilized to generate compounds which are chemical variations of herbs or herbal components. Such compounds can be evaluated using the methods of the present invention.

Basic combinatorial chemist concepts are well known to one of ordinary skill in the chemical arts and can also be found in Nicholas K, Terrett, Combinatorial Chemistry (Oxford Chemistry, Masters), Oxford Univ. Press (1998); Anthony W. Czarnik and Sheila Hobbs Dewitt (Editors), A Practical Guide to Combinatorial Chemistry, Amer. Chemical Society (1997); Stephen R, Wilson (Editor) and Anthony W. Czarnik (Contributor), Combinatorial Chemistry: Synthesis and Application, John Wiley & Sons (1997); Eric M. Gordon and James F. Kerwin (Editors), Combinatorial Chemistry and Molecular Diversity in Drug Discovery, Wiley-Liss (1998); Shmuel Cabilly (Editor), Combinatorial Peptide Library Protocols(Methods in Molecular Biology), Human Press (1997); John P. Devlin, High Throughput Screening, Marcel Dekker (1998); Larry Gold and Joseph Alper, Keeping pace with genomics through combinatorial chemistry; Nature Biotechnology 15, 297 (1997); Aris Persidis, Combinatorial chemistry, Nature Biotechnology 16, 691-693 (1998).

### EXAMPLES

### Example 1

Establishing a Standardized HBR Array for Selected Herbal Compositions.

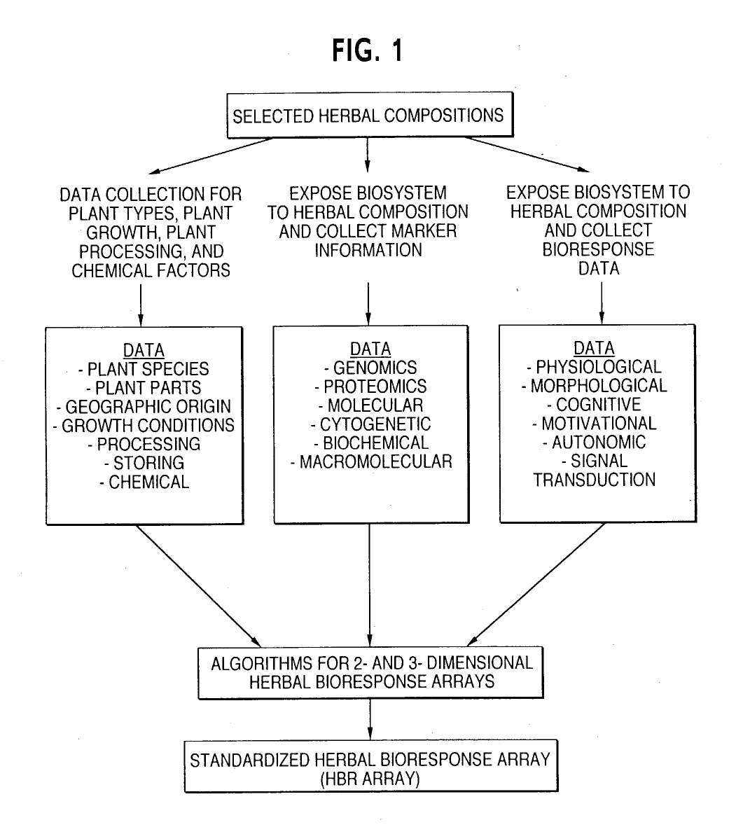

The basic scheme for establishing a Standardized HBR Array is provided in FIG. 1. Definitions of each component of the schematic are provided above.

Following selection of an herbal composition of interest, data is collected for various traits associated with the herbal composition, including, but not limited to plant-related characteristics and marker and BioResponse information.

Plant-related data includes, but is not limited to, the plant species, specific plant parts, geographic origin of the plants in the herbal composition, the growth conditions of the plants, the processing methods used to prepare the herbal components, storage methods and conditions, and various chemical analyses of the herbal composition. Marker information includes qualitative and quantitative data for markers collected after exposure of a biosystem to the herbal compost. Applicable markers include, but are not limited to, molecular markers, cytogenetic markers, biochemical markers and macromolecular markers. BioResponse information includes qualitative and quantitative data for biological responses collected after exposure of a biosystem to the herbal composition.

Each type of data (e.g., chemical, marker, BioResponse) can be obtained using one or more assays on the same, similar, substantially similar, or different batches of the herbal composition of interest. Such different assays can be conducted at the same or different times. In addition, data can be collected for the same or different markers at the same or different times. Similarly, BioResponse data can be collected for the same or different biological responses at the same or different times. Thus, collection of the data for the HBR Array is either collected at one time or collected on an on-going basis. Where a biosystem is exposed to an herbal composition so as to collect data, information is recorded on the administered dosages of the herbal composition as well as treatment times. BioResponse data may also consist of post-translational modifications, such as measurements of signal transduction.

After collection of two or more types of data (e.g., data for two or more markers and a BioResponse; data for plant-related traits and data for a BioResponse), the data is analyzed using algorithms so as to create 2- and/or 3-dimensional Herbal BioResponse Arrays.

Various statistical parameters may be calculated for the HBR Array and may become part of the HBR Array data set. These statistical parameters may include, but are not limited to, means, standard deviations, correlation or match (or mismatch) matrices, ratios, regression coefficients, and transformed values (e.g., arcsin percentage transformations of the raw data). Thus, the HBR Array may consist of the raw data as well as certain calculations, distributions, graphical presentations and other data manipulations associated with the raw data. Particular examples of such information include, but are not limited to, digital images, scatter graphs, cluster analyses and large scale gene expression profiles for marker data.

The total accumulated data and resultant analyses constitute a standardized HBR Array for the particular herbal composition used to establish the HBR Array data set. Due to the iterative nature of the process used to establish and maintain an HBR Array for an herbal composition, such arrays can be viewed as either static at any one point in time or dynamic over time.

The resulting analyses can identify subsets of the standardized HBR Arrays which are correlated (positively or negatively) or associated (i.e., showing a general trend) with one or more specific biological activities of any particular herbal composition.

### Example2

Establishing a Batch HBR Array for Batch Herbal Compositions.

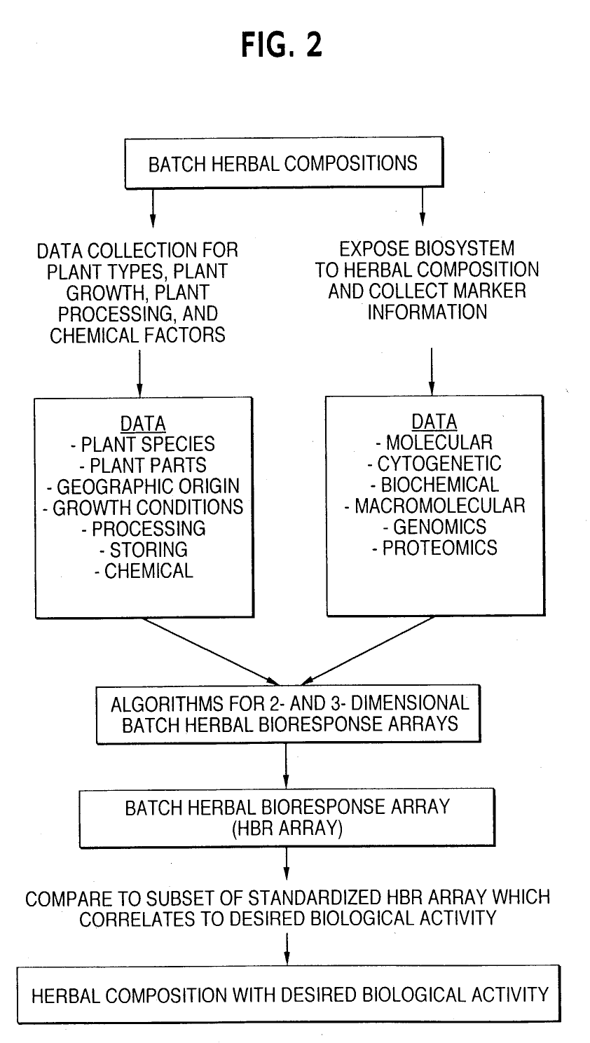

The basic scheme for establishing a HBR Array for a batch of an herbal composition is provided in FIG. 2. Definitions of each component of the schematic are provided above. The procedure for establishing such an array is the same as that set forth immediately above for the standardized HBR Array.

Generally, the amount of data collected for a batch HBR Array will be less than that collected to establish a standardized HBR Array. However, data collected for a batch herbal composition may be added to an established HBR Array or used to establish a new standardized HBR Array.

Generally, the only data collected for a batch herbal composition is that data which has been found to be highly correlated or associated with the desired biological activities of the herbal composition being tested. For example, if it has been determined that a particular subset of plant-related and marker data is highly correlated to a desired biological activity of a particular herbal composition (based on the standardized HBR Array data and analyses discussed above), it is only necessary to test the batch herbal composition for that subset of traits in order to determine whether or not the batch has the desired biological activity. By comparing the data obtained for that subset of traits obtained from the batch (i.e., the batch HBR Array) with the standardized HBR Array for that particular herbal composition, one skilled in the art can determine whether or not that particular batch has the desired biological activity.

### Example 3

Establishing and Using a Major Data Set.

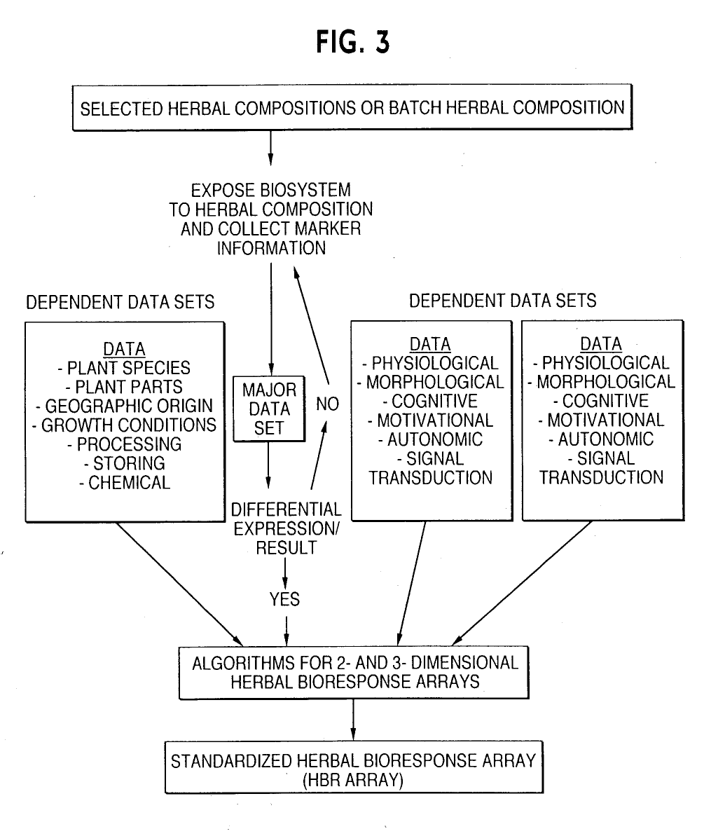

The basic scheme for establishing and using a major data set for an herbal composition is provided in FIG. 3. Definitions of each component of the schematic are provided above.

The first step, is the establishment of a major data set for a selected herbal composition or batch herbal composition. This is accomplished by exposing a biosystem to the herbal composition and collecting the resultant marker information which will constitute the major data set. In most, but not instances, the major data set will consist of genomics and/or proteomics data in the form of an array, such as an array obtained with a DNA biochip.

Next, the major data set is analyzed to see if differential expression/results have been obtained for the tested herbal composition. Differential expression/results are necessary in order to generate meaningful algorithms in the next step. Examples of such differential expression/results include, but are not limited to, indications that certain genes are up- or down-regulated in response to exposure to the herbal composition or that the levels of certain proteins have been increased or decreased in response to the exposure.

If no meaningful or useful differential expression/results are obtained, then it is necessary to repeat the exposure and marker collection step. If it is believed that experimental error lead to the lack of adequate result the first time then the exposure/data collection step can be repeated with all of the variables the same as the first time (e.g., same biosystem, same marker set, same experimental protocol, etc.). However, it may be necessary to vary the biosystem sampling (e.g., type of cell utilized, stage of cell growth), use a different marker set and/or change the experimental protocol in order to get differential expression/result.

### Example 4

Using HBR Array Information.

The HBR Array information discussed herein can be used for many different purposes including, but not limited to, the following: 1) evaluating the components of an herbal composition; 2) predicting the BioResponse of an herbal composition; 3) determining which marker information is most highly correlated with a particular BioResponse of an herbal composition; 3) determining what data set of information (i.e., plant-related data, marker data, and BioResponse data) is most correlated with a particular BioResponse of an herbal compost; 4) determining which type of-biosystem is best for evaluating the biological activity of an herbal composition; 5) adjusting or changing the components of a herbal composition so that the HBR Array of that herbal composition corresponds to a standardized HBR Array for the same or substantially the same herbal composition; 6) adjusting or changing the components of an herbal composition so that the herbal composition will have the desired biological activity; 7) measuring the relatedness of different herbal compositions; 8) creating and updating standardized HBR Arrays; 9) identifying specific components (e.g., plant parts, proteins, molecules) which retain the desired biological activity of an herbal composition; 10) determining which components of an herbal composition can be eliminated while maintaining or improving the desired biological activity of the herbal composition; 11) identifying one or more previously unknown biological activities for an herbal composition; 12) aiding in the design of therapeutics which include herbal and non-herbal components, such as chemically-synthesized drugs or pharmaceuticals and 13) utilizing the HBR Array information to complement combinatorial chemistry methods of designing therapeutics. Each of these embodiments of the present invention can be accomplished by one skilled in the applicable art using the methods and tools provided herein.

### Example 5

Quality Control.

The HBR Array technology of the present invention is used to correlate or to determine a substantial equivalence of a specific batch of an herbal composition (single herb or multiple herbs of a formula) to a standardized, or master, batch of a same or substantial similar herbal composition. The HBR Arrays utilized in this process include the acceptable range of quantitative variation for each of the biological effects (i.e., BioResponse), and possibly a global score composed of weighted values assigned to each of the biological effects, which may consist of markers from multiple biochemical pathways of a biosystem.

“Data mining” refers to a process used to determine or select which subset of biological effects is the minimum number of biological effects required in any specific HBR Array. The information for data mining results from exposing a biosystem (e.g., a cell line) in a dose dependent manner to a standardized herbal composition to establish a standardized HBR Array. This standardized HBR Array can then be compared to various HBR Arrays established for test herbal compositions. These test herbal compositions include, but are not limited to, different batches prepared at different dates; different batches prepared from raw herbs collected at different times; and different batches prepared from raw herbs collected at different locations.

### Example 6

Improving an Herbal Composition or Identifying New Uses for an Herbal Composition.

HBR Arrays are generated by exposing biosystems to either extracts from individual herbs of a formula, or to extracts from the whole formula, and examining the biological effects of the extracts. The observed biological effects can be from multiple biochemical pathways of a biosystem and/or from multiple tissues of an animal, wherein various markers are evaluated for their corresponding qualitative and or quantitative changes. The resulting HBR Arrays can be compared to novel HBR Arrays or to similar HBR Arrays from different herbal compositions or herbal compositions prepared by different processes. This procedure is useful for selecting a given set of biological effects and the minimum number of markers required to predict that a given batch herbal composition has the given set of biological effects.

In order to construct HBR Arrays, one skilled in the art utilizes various data mining tools including, but are not limited to statistical analyses, artificial intelligence, and database research on neural work. The statistical methods of choice include, but are not limited to, basic exploratory data analysis (EDA), graphic EDA (such as bushing) and multivariate exploratory techniques (e.g., cluster analysis, discriminating factor analyses, stepwise linear on non-linear regression, classification, tree (see, e.g., STATISTICA™, software packages from StatSoft, Tulsa, Okla. 74104; Tel: 918-749H1119; Fax: 918-749-2217; www.statsoft.com).

Data mining tools are used to explore large amounts of HBR Array data in search of constructing an HBR Array and consistent pattern within, between or among various HBR Arrays. The procedure consists of exploration, construction of an HBR array, and validation. This procedure is typically repeated iteratively until a robust HBR Array, or standardized HBR Array, is identified.

### Example 7

Establishing a Standardized HBR Array for Ginseng Recipes.

For the purposes of this example, standard ginseng is chosen to be Panax Ginseng C. A. Meyer G115 grown either in Manchuria or in Korea. The climate for growth is between −10 to +10° C. with an annual rainfall of 50-100 cm (see Huang in The Pharmacology of Chinese Herbs, (1993) pp 21-45, CRC Press, Boca Raton, Fla., fully incorporated by reference). Ginseng batches will first be characterized by geographic origin, species, plant part (e.g., rhizome, root, leaf skin, seed, bud and flower); growth conditions, processing methods and storage conditions both before and after processing. Verification of chemical content for these batches will be performed by qualitative HPLC analysis for determination of ginsenoside saponins (e.g., Ro, Ra1, Ra2, Rb1, Rb2, Rb3, Rc, Rg1, Rg2, Rd, Re, Rf, Rh1, Rh2, NG-R2 and Z-R1), including TLC qualitative analysis for lipophilic constituents (see, Elkin et al., Chumg Kuo Yao Li Hsueh Pao (1993) 14: 97-100 and Yoshikawa et al., Yakugaku Zasshi (1993) 113: 460-467). The saponin content of different herbs should be between 2.1 and 20.6% (by weight) depending on the species (see Table 1). These data will then be stored, preferably in the memory of a computer processor, for further manipulation.

TABLE 1
Saponin Content of Different Ginseng Herbs.*

| Species | Total saponins (% by weight) |
|---|---|
| Panax ginseng C. A. Meyer | 2.1-4.4% |
| Panax quiquefolius | 4.9% |
| Panax notoginseng and Panax japonica | 13.6-20.6% |
| Panax japonica var. major | 9.34% |

*from Huang in The Pharmacology of Chinese Herbs, (1993) page 29, CRC Press, Boca Raton, FL.

Expression biomarkers for standard ginseng (i.e., G115) include the following: IL-8, 1L-2, GM-CSF, NFκB, ICAM-1, interferon gamma, choline acetyl transferase, trk A, nerve growth factor (Kim et al., Planta Med (1998) 64: 110-115; Sonoda et al., Immunopharmacology (1998) 38: 287-294; Baum et al., Eur J Appl Physiol (1997) 76: 165-169; Iwangawa et al., Free Radic Biol Med (1998) 24:1256-1268; Rhind et al., Eur J Appl Physiol (1996) 74: 348-360). Alternatively, for a broader batch size, the 400,000 oligonucleotide group/1.6 cm² chip of Affymetrix can be used (U.S. Pat. No.5,556,752). The expression biomarkers for standard ginseng will be prepared by nucleic acid microarray technology using either photolithography, mechanical microspotting or ink jet application (see Schena et al., TIBTECH (1998) 16: 301-306). Selected sets of cells will be contacted with standard ginseng for varying periods of time, under varying conditions to generate multiple microarray sets. The microarray sets will then be analyzed by hybridization-based expression monitoring of biochemical extracts via deduction of steady state mRNA levels from fluorescence intensity at each position on the microarrays (Schena et al., Science (1995) 270: 467-470; Schena et al., Proc Natl Acad Sci USA (1996) 93: 10614-10619; Lockhart et al. Nat Biotechnol (1996) 14: 1675-1680; DeRisi et al., Nat Genet (1996) 14: 457-460; Heller et al., Proc Natl Acad Sci USA (1997) 94: 2150-2155). The array data sets are then input into algorithms to generate statistical expression biomarker values for standard ginseng. Biochemical biomarkers for standard ginseng include quantitative analysis for increases in cycloheximide sensitive.[³H]-leucine incorporation proportional to protein synthesis and [³H]-thymidine incorporation reflective of mitosis. (see Yamamoto et al., Arzneimittelforschung (1977) 27: 1169-1:173). For biochemical biomarkers, bone marrow cells will be contacted with standard ginseng for varying time periods under varying conditions in the presence of [³H]-thymidine (for DNA synthesis) or in the presence and absence of cycloheximide and [³H]-leucine (for protein synthesis) to perform multiple quantitative analysis of biochemical biomarkers (i.e., BBM sets) The BBM sets are then input into algorithms to generate statistical biochemical biomarker values for standard ginseng. Statistical data will then be stored, preferably in the memory of a computer processor, for further manipulation.

Biological response of a biosystem (i.e., BioResponses) will be determined using cells and whole animals. For cells, ginseng batches will be exposed to specific cell types, including, but not limited to, fibroblasts, macrophages, monocytes, PMNL, LAK cells, B16-F10 melanoma cells, THP-1 cells and hippocampal neurons at a concentration of 0.5 mg/ml to 100 mg/ml. For animal treatments, 0.5-100 mg/kg of ginseng herbal extract will be administered orally, by intraperitoneal injection or subcutaneous injection.

To determine a biological response of a biosystem to standardized ginseng, human ovarian cancer cells will be inoculated into nude mice, which results in the formation of palpable tumors. After tumor formation the mice will be treated by co-administration of cis-diamminecichloroplatinum and standard ginseng. Mice will be examined for tumor growth inhibition, increase in survival time and lowered adverse side-effects on hematocrit values and body weight (Nakata et al., Jpn J Cancer Res (1998) 89:733-740). The assay will be repeated using various concentrations of standard ginseng to generate measures of central tendency, dispersion and variability for each variable.

The data collected will then be subjected to multidimensional analysis to generate multivariant normal distribution sets as a means of determining a baseline correlation between biological activity and standard ginseng (see Zar, J. H., in Biostatistical Analysis, 2nd ed. (1984), pp 328-360, Prentice Hall, Englewood Cliffs, N.J.). A second independent determination of a biological response of a biosystem to standard ginseng will be the effect of standard ginseng on physical performance during exercise. Rats will be treated for 4 days with standard ginseng at various concentrations (between 0.5-100 mg/kg/day) and animals will be tested for increased plasma free fatty acid level and maintenance of glucose level during exercise at approximately 70% VO2max (see Wang et al, Planta Med (1998) 64130-133). The data generated will be collected and then subjected to multidimensional analysis to generate multivariant normal distribution sets as a means of determining a baseline correlation between biological activity and standard ginseng (see Zar, J. H., in Biostatistical Analysis. 2nd ed. (1984), pp 328-360, Prentice Hall, Englewood Cliffs, N.J., Herein, fully incorporated by reference). The distribution sets for each BioResponse are then put into algorithms to generate statistical values for standard ginseng. Statistical data will then be stored, preferably in a memory of a computer processor, for further manipulation.

Each of these steps (ie., chemical analysis, generation of biomarker information and determination of responses of a biosystem) is reiterated to generate a large database of statistical values. These values are compiled and input into an algorithm to generate 2- and 3-dimensional Herbal Response Arrays (HBR Array) for standardized ginseng. Through reiteration, the resulting arrays (i.e., Standardized Arrays) display the highest correlation between composition (including growth conditions), biomarker information and biological response for standardized ginseng. By determining two or more known associated variables for composition and biomarker information values via display on an HBR Array for a test batch, the values for biological response variables can be predicted for the test batch by comparing test values against Standardized HBR Array values for standardized ginseng. The resulting prediction will be used to evaluate the quality of a given ginseng batch without necessitating the use of an observed biological response of a biosystem (see Example 2).

### Example 8

Evaluation of a Selected Herbal Composition of Ginseng Using a Subset of Variables Correlated with a Specific Biological Response.

To evaluate the quality of a test batch herbal composition, data is first collected concerning the plant-related parameters for the herbs in the selected herbal composition (e.g., plant species, plant parts, geographic origin, growth conditions, processing methods and storage conditions). The selected herbal composition is then manipulated such that chemical analysis can be performed to determine the chemical content of the herb (see Elkin et al., Chumg Kuo Yao Li Hsueh Pao (1993) 14: 97-100 and Yoshikawa et al., Yakugaku Zasshi (1993) 113: 460-467). Previously obtained ginseng data has demonstrated a strong correlation between oxygen consumption during aerobic exercise performance and the presence of a subset of saponin components, especially Rg1 and Rb1 (Wang et al., Planta Med (1998) 64: 130-133).

The test batch is then exposed to test cells including, but not limited to, fibroblasts, macrophages, monocytes, PMNL, LAK cells, B16-F10 melanoma cells, THP-1 cells and hippocampal neurons at a concentration of 0.5 mg/ml to 100 mg/ml to determine expression biomarker values. mRNA is isolated from exposed cells which is subsequently manipulated to serve as a substrate for hybridization-based expression monitoring of biochemical extracts using microarrays comprising IL-8,-IL-2 and Interferon gamma cDNA (Schena et al., Science (1995) 270: 467-470; Schena et al., Proc Natl Acad Sci USA (1996) 93: 10614-10619; Lockhart et al., Nat Biotechnol (1996) 14: 1675-1680; DeRisi et al., Nat Genet (1996) 14: 457-460; Heller et al., Proc Natl Acad Sci USA (1997) 94: 2150-2155). Previously obtained ginseng data has demonstrated a strong correlation between oxygen consumption during aerobic exercise performance and the induction of the expression biomarkers IL-8, IL-2 and Interferon gamma in test cells (Venkatraman et al., Med Sci Sports Exerc (1997) 29: 333-344 and Wang et al., Planta Med (1998) 64: 130-133). For biochemical biomarkers, rat bone marrow cells will then be exposed to the test batch and assayed for [³H]-thymidine incorporation reflective of mitosis. Previously obtained ginseng data has demonstrated that Rb1 and Rg1 show a strong correlation with DNA synthesis in rat bone marrow cells (Yamamoto et al., Arzneimittelforschung (1978) 28: 2238-2241).

After reiterative analysis, data from each assay will be input into an algorithm to generate a test HBR array for the selected herbal composition based on the enumerated plant-related data, including chemical analyses, and data concerning the subset of biomarkers. The quality of a test batch will be determined by comparing test HBR and standard ginseng Standardized HBR Array variables directed toward analysis of the above observations and subsets, wherein the demonstration of the induction of IL-2, IL-8 and INF gamma mRNA in vitro and an increase in [³H]-thymidine incorporation in rat bone marrow cells (including data collected on growth conditions, origin, and verification of the saponins Rg1 and Rb1) is predictive of an equivalent BioResponse effect of the test batch on oxygen consumption as that exhibited by standard ginseng. Based on this procedure it can be determined whether or not the test batch is of a similar or different quality than that of the standard for the given biological response or biological response of interest.

### Example 9

Establishing a Standardized HBR Array for Huang Ling (HL) Recipes.

For the purposes of this example, standard huang ling (HL) is chosen to be Coptis chinesis France, from southwest Asia, wherein growth conditions are well known to one skilled in the art (see Huang in The Pharmacology of Chinese Herbs, (1993), pp 69 and 287-288, CRC Press, Boca Raton, Fla.). Dried rhizomes of Coptis chinesis France will be verified for chemical content by quantitative chemical analysis for determination arsenic, berberine, caeruleic acid, columbamine, copsine, coptine, coptiside-I, coptiside-II, coptisine, coreximine, epiberberine, ferulic acid, greenlandicine, isocoptisine, lumicaerulic acid, magnoflorine, oxybererine, thalifendine, umbellatine, urbenine, worenine, palmatine, jatrorrhizine and colubamine (see also Zhu M., Chung Yao Tung Pao (1984) 9: 63-64). Content of the alkaloid berberine of different herbs should be between 7-9% (by weight). These data will be stored, preferably in the memory of a computer processor, for further manipulation.

Expression biomarkers for standard HL include the following: NfκB; bcl-2 analog, A1; zinc finger protein, A20; IL-2receptor; cell cycle probes; c-Ki-ras2; growth regulators probes and glucocorticoid receptor dependent apoptosis probes (see Chi et al., Life Sci (1994) 54: 2099-2107; Yang et al., Naunyn Schmiedebergs Arch Pharmacol (1996) 354: 102-108; Miura et al., Biochem Pharmacol (1997) 53; Chang K. S., J Formos Med Assoc (1991) 90: 10-14). Alternatively, for a broader batch size, the 400,000 oligonucleotide group/1.6 cm² chip of Affymetrix can be used (U.S. Pat. No.5,556,752). The expression biomarkers for standard HL will be prepared by microarray technology as described in Example 1, including analysis and statistical data generation. Biochemical biomarkers for standard HL include increase in glucocorticoid receptor and inhibition of alpha-fetoprotein secretion in HL exposed HepG2 cells (see Chi et al., Life Sci (1994) 54: 2099-2107). BBM sets are generated and analyzed as described in Example 1. Statistical data will then be stored, preferably in the memory of a computer processor, for further manipulation.

Biological response of a biosystem will be determined using cells and whole animals. Batches of the selected herbal composition will be exposed to specific cell types, including but not limited to, human HepG2 hepatoma cells, human embryonal carcinoma cells and thymocytes at concentrations from 0.1-100 mg/ml. For animal treatments 0.1 mg-2 g/kg of coptic herbal composition (i e., HL) will be administered orally, by intraperitoneal injection or subcutaneous injection. To determine a biological response of a biosystem to standardized HL, human embryonal carcinoma clone, NT2/D1 is exposed to various concentrations of standard HL and cells will be examined for differentiation into cells with neuronal-like cell morphology (Chang K. S., J Formos Med Assoc (1991) 90: 10-14). The assay will be repeated to generate measures and analysis will be performed as described for ginseng in Example 1. A second independent determination of a biological response of a biosystem to standard HL will be the effect of standard HL on diarrhea due to enterotoxigenic Escherichia coli (ETEC). Patients with active diarrhea due to ETEC will be treated with various concentrations of HL (e.g., 2 g/kg) and stool volumes will be determined (see, e.g., Rabbani G. H., Dan Med Bull (1996) 43:173-185). The assay will be repeated to generate measures and analysis will be performed as described for ginseng in Example 1. The distribution sets for each biological system are then put into algorithms to generate statistical values for standard HL. Statistical data will then be stored, preferably in the memory of a computer processor, for further manipulation.

Lastly, as in Example 1, the steps are reiterated to generate HBR arrays for standardized HL, wherein the resulting HBR arrays will then be used to predict biological activity and evaluate batch quality. Using this method, a Standardized HBR Array can be generated and updated periodically.

### Example 10

Evaluation of a Selected Herbal Composition of Huang Ling Using a Subset of Variables Correlated with a Specific Biological Response.

To evaluate the quality of a selected test batch of an herbal composition of Huang Ling, data is first collected concerning the plant-related characteristics (e.g., plant species, plant parts, geographic origin, growth conditions, processing methods and storage conditions). The herbal composition is then manipulated such that chemical analysis can be performed to determine the chemical content of the composition (see also Zhu M., Chung Yao Tung Pao (1984) 9: 63-64).

Previously obtained HL data has demonstrated terminal differentiation of human embryonal carcinoma clones into neuronal-like cells is strongly correlated with the presence of berberine (see Chang K. S., J Formos Med Assoc (1991) 90: 10-14). The test batch is then exposed to test cells including human embryonal carcinoma clone, NT2/D1 at a concentration starting at a non-toxic concentration (determination of which is within the skill of the ordinary artisan). mRNA is isolated from exposed cells which is subsequently manipulated to serve as substrate for hybridization based expression monitoring of biochemical extracts using microarrays comprising IL-2 receptor and NFκB; (see Chi et al., Life Sci (1994) 54: 2099-2107; Yang et al., Naunyn Schmiedebergs Arch Pharmacol (1996) 354:102-108; Miura et al., Biochem Pharmacol (1997) 53; Chang K. S., J Formos Med Assoc (1991) 90: 10-14; U.S. Pat. No. 5,556,752), and which can be used to determine down regulation of c-Ki-ras2 gene expression in said cells. Previously obtained HL data has demonstrated terminal differentiation of human embryonal carcinoma clones into neuronal-like cells is strongly correlated with induction of mitogen probes and down regulation of c-Ki-ras2 gene expression (see Chang K. S., J Formos Med Assoc (199i) 90: 10-14).

For biochemical markers, HepG2 cells are exposed to the test composition and cells are assayed for increase in glucocorticoid receptor and inhibition of alpha-fetoprotein secretion (see Chi et al., Life Sci (1994) 54:-2099-2107). Previously obtained HL data has demonstrated that inhibition of glucocorticoid induced apoptosis is strongly correlated with berberine-type alkaloids (see Miura et al., Biochem Pharmacol (1997) 53: 1315-1322). After reiterative analysis, data from each assay will be input into an algorithm to generate a test HBR array based on the enumerated observational data, chemical data and data concerning the subset of biomarkers.

The quality of a test batch will be determined by comparing test HBR and standard HL HBR Array variables directed toward analysis of the above observations and subsets, wherein the demonstration of the induction of IL-2 receptor and NFB, the down regulation of c-Ki-ras2 gene expression, an increase in glucocorticoid receptor and inhibition of alpha-fetoprotein secretion for HepG2 cells (to including data collected on growth conditions, origin, and verification of berberine alkaloid) is predictive of an equivalent BioResponse effect of the test batch on terminal differentiation of human embryonal carcinoma clones into neuronal-like cells and inhibition of dexamethasone induced apoptosis as that exhibited by standard HL. Based on this procedure it can be determined whether or not the test batch is of a similar or different quality than that of the standard.

### Example 11

Evaluation of Xiao Chai Hu Tang (Sho-Saiko-To) Using Two Bioassays.

To evaluate the quality of thee sources of Xiao Chai Hu Tang, two bioassays were used: 1) cell growth inhibition and 2) hepatitis B virus secretion from infected cells. The Xiao Chai Hu Tang composition is made from a mixture of 6-7 herbal plants (Radix Bupeuri, Rhizoma Pinelliae, Rhizoma Zingiberis, Radix Scutellariae, Fructus Ziziphi, Codonopsis Pilosula, Radix Ginseng and Radix Glycyrrhizae, see Table 2 for relative amounts, by weight).

TABLE 2
Composition of Xiao Chai Hu Tang.

| | Plant Species | | | | | | | |
|---|---|---|---|---|---|---|---|---|
| | Radix | Rhizoma | Rhizoma | Radix | Fructus | Codonopsis | Radix | Radix |
| | bupleuri | pinelliae | zingiberis | scutellariae | ziziphi | pilosula | ginseng | glycyrrhizae |
| Source | Relative Amount by Weight | | | | | | | |
| Singapore | 1 | 1 | 0.375 | 0.375 | 0.375 | — | 0.375 | 0.375 |
| Korea | 1 | 0.717 | 0.571 | 0.492 | — | 0.429 | — | 0.288 |
| Taiwan | 1 | 0.25 | 0.375 | 0.375 | 0.25 | — | 0.375 | 0.375 |

The three “recipes” originate in either Singapore, Korea or Taiwan. Batches were evaluated for toxicity and for the ability to inhibit hepatitis B virus as detected by DNA quantitation or detection of hepatitis B surface antigen (HbsAg) (see Dong et al., Proc Natl Acad Sci USA (1991) 88: 8495-8499).

Briefly, one gram of preparation was added with 10 ml of water. The mixture was boiled for 30 minutes. The supernatant was collected after centrifugation and filtered through a 0.22 μm filter. Two cell types were used: a) 2.2.15 cells which secrete hepatitis B virons (kindly provided by Professor G. Ace; see Ace et al. Proc Natl Acad Sci USA (1987) 84: 1005-1009) and b) HepG2 cells (ATCC cat #HB-8065). One to fifty dilutions were used for each assay. The cell growth inhibition assay was performed for 72 hours. All other procedures were performed as described by Dong et al., Proc Natl Acad Sci USA (1991) 88: 8495-8499. The results of the assays using the three batches is displayed in Table 3. Based on these data, the Taiwan source would be selected as a standard herbal composition because of its low toxicity combined with its effectiveness in reducing secretion HbsAG (which is proportional to viral release) by more than half.

TABLE 3
Bioassay of Xiao Chai Hu Tang (Sho-saiko-to).

| | |
|---|---|
| | Hepatitis B |
| Cell Growth | Virus (secreted) |
| Inhibition (%) | % Inhibition |

| Source | HepG2 | 2.2.15 | DNA | HbsAG |
|---|---|---|---|---|
| Singapore | 73 | 100 | 65 | 38 |
| Korea | 13 | 60 | 20 | 42 |
| Taiwan | 0 | 42 | 0 | 47 |

The data presented in Tables 2 and 3 for the Taiwan herbal composition constitute the initial data for the standardized HBR Array for this herbal composition. Therefore, this data set would initially include the source of the herbal composition, the plant species and relative amounts of each the herbal composition, and two BioResponses (i.e., cell growth inhibition and hepatitis B virus secretion from infected cells).

Using the procedures set forth in the schematic of FIG. 1 and in Examples 1 and 3, additional data can be collected on plant-related data, markers and BioResponses for the standard herbal composition. This additional data is added to the initial standardized HBR Array to generate an expanded standardized HBR Array. Appropriate analyses of the resulting database can be conducted as set forth in the detailed description and the examples in order to ascertain the subset of variables which is most highly correlated or associated with the BioResponse of interest. Batch HBR Arrays may be determined using the methods depicted in FIG. 2 and in the procedures of Examples 2 and 4.

The resultant batch HBR Array can be compared to the standardized HBR Array so as to predict the BioResponse of the batch herbal compositions.

### Example 12

Herbal Preparation

The standardized protocol for the herbal extract preparation was as follows: The ingredients of herbal raw materials with proper ratios were placed in a jacketed reactor and extracted with water at an elevated constant temperature with mixing. The solid was separated from the liquid with a 120-mesh screen. The resultant filtrate was collected and then concentrated by evaporating the water under reduced pressure. The concentrated liquor was spray dried at elevated temperature to yield granulated powder. This bulk substance was then formulated into the desired dosage form.

### Example 13

Evaluation of Huang Qing Tang

Huang Qing Tang (HQT) is an ancient Chinese botanical formula composed of four distinct herbs: Scutellariae (scute), Glycyrrhizae (licorice), Paeonie lactiflora pallus (white peony root), and Fructus zizipho (date). (Table 4). This herbal formula has been long used in Asia to treat a variety of gastrointestinal ailments since 300 AD.

TABLE 4
Herbal Ingredients of TCM Formula HQT

| Scientific Name | Common Name | Traditional Use |
|---|---|---|
| Scutellariae | Scute Baical | Used to reduce capillary |
| Radix | Skullcap root | permeability: to reduce inflammation: |
| | | to treat enteritis and |
| | | dysentery: increase the |
| | | secretion of bile to treat |
| | | jaundice: to relieve muscle |
| | | spasms to treat coughing: |
| | | to expel parasites. |
| Glycyrrhizae | Licorice Root | Used to moisten the |
| Radix | | lungs and stop coughs: to relax spasm |
| (Gancao) | | and stop pain: to moderate |
| | | the action of herbs; to |
| | | reduce fire and release toxins. |
| Fructus Ziziphi | Date | Has diuretic and |
| | | strengthening effects. |
| Paeonie lactiflora | White Peony | Used to suppress and soothe |
| pallus radix | Root | pain; to soothe ligaments |
| | | and purify the blood. |

Biological and Enzyme Assays

TABLE 5
Batch Properties (HQT)

| Property | Batch A | Batch B | Batch C |
|---|---|---|---|
| Origin | Taiwan, Sun-Ten | Taiwan, Sun-Ten | Taiwan, Sun-Ten |
| Preparation | Standard | Standard | Boiled 30 min. |
| method | | | |
| Plant part | Root | Root | — |

Briefly, one gram of each batch of Huang Qing Tang (HQT) was added with 10 ml of water (1 mg/ml). The mixture was treated as outlined in Table 5. The supernatant was collected after centrifugation and filtered through a 0.22 μm filter. Two cell types were used to test for biological effects of each batch of HQT: a) Jurkat T cells (ATCC cat #TIB-152) and b) HepG2 cells (ATCC cat #HB-8065). One to fifty dilutions were used for each assay. Frozen cells (10⁷/ml) were quickly thawed in a water bath at 37° C. The cells were then diluted in 10 ml of pre-warmed media (see Life Technologies, Inc., Catalogue and Reference Guide, 1998-1999, Cell Culture section) followed by centrifugation at 1500 rpm for 5 min. The supernatant was then discarded and the cells were cultured in 100 ml media at 37° C., 5% CO₂. After 2 days, the cells were counted (approximately 8×10⁵/ml, total 100 ml).

Batches were also evaluated for the ability to inhibit hepatitis B virus as detected by DNA quantitation (see Dong et al., Proc Natl Acad Sci USA (1991) 88: 8495-8499). Briefly, one gram of preparation was added with 10 ml of water. The mixture was boiled for 30 minutes. The supernatant was collected after centrifugation and filtered through a 0.22 μm filter. HepG2.2.15 cells which secrete hepatitis B virons (kindly provided by Professor G. Ace, see Ace et al. Proc Natl Acad Sci USA (1987) 84: 1005-1009) were used in this assay. One to fifty dilutions were used for each assay. The cell growth inhibition assay was performed for 72 hours. All other procedures were performed as described by Dong et al., Proc Natl Acad Sci USA (1991) 88: 8495-8499.

β-glucuronidase was assayed as HQT is known for its anti-diarrhea properties. Different HQT extracts were added to triplicate wells of a 96-well plate which contained 0.1 mM phenolphthalein glucuronidate, 70 mM Tris-HCl (pH 6.8) and 0.8 ng of dialyzed β-glucuronidase (from E. Coli purchased from Sigma™) to a final volume of 80 μl. After 2 hr incubation at 37° C., the reactions were terminated with 200 μl of stopping solution which contained 0.2 M Glycine and 0.2 M NaCl (pH 10.4), and the OD was monitored with a kinetic microplate reader at 540 nm.

The results of the assays using the three batches are displayed in Table 6. Based on these data, HQT sources A and B have relatively low toxicities combined with higher inhibitory activity relative to batch HQT C (i.e., approximately 5 fold greater toxicity toward HepG2 cells and 3.3 fold less inhibitory activity against β-glucuronidase than either HQT A or B, see Table 6).

TABLE 6
Biological Assay of Three Preparations of HQT*

| | E. Coli | | | HBV‡ |
|---|---|---|---|---|
| | β-Glucuronidase | HepG2 | Jurkat | DNA |
| HQT A | 0.6 | 1.50 | 0.76 | None |
| HQT B | 0.7 | 1.6 | 0.81 | ND |
| HQT C | 2.2 | 0.32 | ND | ND |

*Values represent IC₅₀ values.
‡% of Control
ND, not determined.

Evaluation of HQT Effects on Protein Expression

HepG2 cells (1×10⁶) were seeded in 25 cm² flasks in 3.0 ml of RPMI-1640 medium (see Life Technologies, Inc., Catalogue and Reference Guide, 1998-1999, Cell Culture section) 24 hr before the drug addition. The cells were treated with or without herbal medicine, where the former is added at two final concentrations of 0.2 mg/ml or 4 mg/ml, respectively, and incubated at 37° C. for 24 hours. The medium was removed and the cells were washed twice with cold PBS. The cells were harvested into 1 ml of PBS and centrifuged at 10,000 rpm for 2 minutes, extracted on ice with a buffer containing 50 mM Tris-Cl (pH 7.5), 0.2 mM PMSF and 10% glycerol, followed by three freeze-thaw cycles. Potassium chloride was added to the cell lysate at a final concentration of 0.15 M prior to centrifugation. The protein concentration was determined and the cell extract was electrophoresed according to the method of Laemmli (Nature (1970) 227:680-685). Western blots were performed by standard techniques known in the art, see for example Sambrook, et al (1989). The antibodies used were directed to the following proteins: Topo I, Stat (20707); Cyclin B1; MAPK (Ab2) and Nm 23 H1.

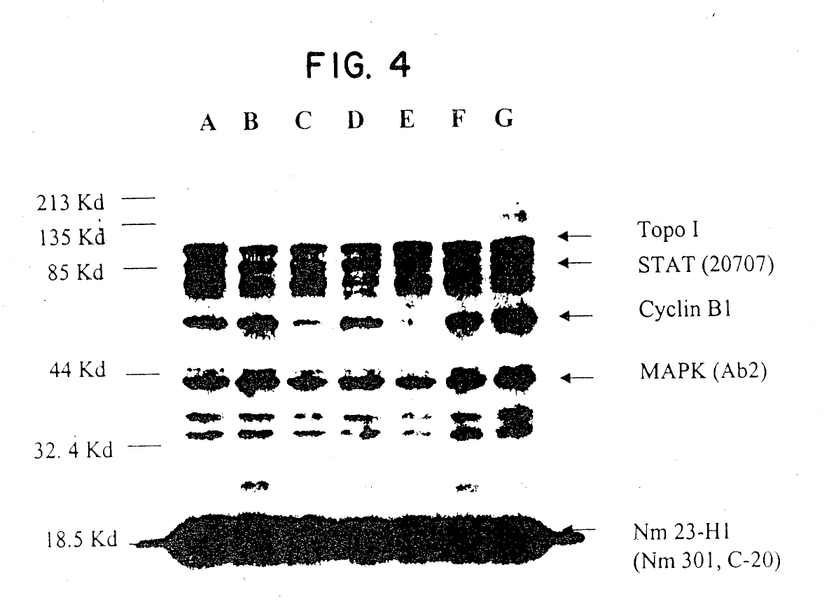

FIG. 4 demonstrates that the higher concentrations of HQT A or HQT B differentially effects the expression of cyclin B1 polypeptide.

HPLC Analysis

The herbal batches were analyzed by HPLC with a Beckman ODS Ultrasphere™ column (5 micron particles, 4.6 mm×25 cm) and detected with an UV spectrophotometer (Perkin Elmer). The wavelengths for UV detection were monitored at 280 nm and 340 nm. The mobile phase was pumped at 1 ml/min and consisted of Solvent A: H₂O and Solvent B: 20% MeOH with the following gradient: 1) the solvent was 100% solvent A for the first 5 minutes; 2) the solvent composition was changed to 10% solvent A/90% solvent B for the next 10 minutes; and 3) the solvent was changed to 10% solvent A/90% solvent B for the next 40 minutes. This was followed by the addition of 100 % solvent A for 5 minutes. The HPLC markers are baicalin and baicalein.

Mass Spectrometry

The herbal extract was analyzed by Mariner™ ESI-TOF Mass Spectrometry (MS) from PE Biosystems. Control tracings were generated using baicalein and baicalin, two known active ingredients in HQT.

HQT samples in water and acid treated batches were been analyzed by HPLC and Mass Spectrometry. While water treated HQT batches A and B had distinct TPLC and MS tracings, acid treated batches gave almost identical patterns (data not shown).

Algorithm

The data collected form part of the multidimensional analysis used to generate multivariant normal distribution sets as a means of determining a baseline correlation between biological activity and standard HQT chemical (HPLC and Mass Spec), and origin/growth characteristics.

### Example 14

Individual Components

A. Licorice.

Evaluation of Glycyrrhizae Radix (licorice)

Licorice is useful for moistening the lungs and reducing coughs, helps to relax spasm and pain. The properties of the licorice batches used in this example are presented in Table 7.

TABLE 7
Batch Properties (licorice)

| Property | Batch A | Batch B | Batch C | Batch D |
|---|---|---|---|---|
| Plant Name | Glycyrrhizae | Glycyrrhizae | Glycyrrhizae | Glycyrrhizae |
| | Radix | Radix | Radix | Radix |
| Origin | Inner | Inner | U.S., Kin | U.S., Kin Man |
| | Mongolia | Mongolia | Man Herb | Herb Center |
| | | | Center | |
| Preparation | Standard | Standard | Boiled | Warm H₂O, |
| method | | | 30 min. | 30 min. |
| Plant part | Root | Root | — | — |

Biological and Enzyme Assays

To assay the quality, of herbal sources, each herbal extract supernatant was assayed and the analysis was repeated three times. For a given sample to be assayed, 1 gram of herbal powder was dissolved in 10 ml of 80° C. deionized water (neutral pH) in a polypropylene tube. The tube was then incubated as outlined in Table 7, then centrifuged to obtain the supernatant. Batches of licorice were tested against either HepG2 cells (ATCC cat #HB-8065) or Jurkat T cells (ATCC cat #TIB-152) or both. Cells were cultured for 24 hours as described above.

Batches were also evaluated for the ability to inhibit hepatitis B virus as detected by DNA quantitation (see Dong et.al., Proc Natl Acad Sci USA (1991) 88: 8495-8499). Briefly, one gram of preparation was added with 10 ml of water. The mixture was boiled for 30 minutes. The supernatant was collected after centrifugation and filtered through a 0.22 μm filter.2.2.15 cells which secrete hepatitis B virons (kindly provided by Professor G. Ace; see Ace et al. Proc Natl Acad Sci USA (1987) 84: 1005-1009) were used in this assay. One to fifty dilutions were used for each assay. The cell growth inhibition assay was performed for 72 hours. All other procedures were performed as described by Dong et al., Proc Natl Acad Sci USA (1991) 88: 8495-8499.

Again, β-glucuronidase was assayed. Different licorice extracts were added to triplicate wells of a 96-well plate which contained 0.1 mM phenolphthalein glucuronidate, 70 mM Tris-HCl (pH 6.8) and 0.8 ng of dialyzed beta-glucuronidase (from E. Coli, purchased from Sigma) to a final volume of 80 μl assayed as above.

The results of the assays using the two batches is displayed in Table 8. Based on these data, licorice batch A was much more toxic to Jurkat cells than batches B (approximately 9 fold) and a more effective inhibitor of β-glucuronidase (see Table 8).

TABLE 8
Biological Assay of Four Preparations of Licorice*

| | E. Coli | | | HBV‡ |
|---|---|---|---|---|
| | β-Glucuronidase | HepG2 | Jurkat | DNA |
| Licorice A | 1.1 | 1.07 | 0.41 | None |
| Licorice B | ND | ND | 3.6 | ND |
| Licorice C | 2.1 | ND | ND | ND |
| Licorice D | ND | ND | >2.0 | 53.8 |

*Values represent IC₅₀ values.
‡% of Control
ND, not determined.

Expression Assay

In order to assay gene expression, Jurkat T cells were treated with herbal extract as follows: Jurkat cells (10⁷/ml) were quickly thawed in a water bath at 37° C. The cells were then diluted in 10 ml of pre-warmed media (see Life Technologies, Inc., Catalogue and Reference Guide, 1998-1999, Cell Culture section) followed by centrifugation at 1500 rpm for 5 min. The supernatant was then discarded and the cells were cultured in 100 ml media at 37° C., 5% CO₂. After2 days,,the cells were counted (approximately 8×10⁵/ml, total 100 ml).

The herbal extract solution was prepared as outlined above (e.g., 2 g of an herbal powder to obtain 20 ml of sterile solution (0.1 g/ml). The cells were divided into 3 flasks at a density of 2.5×10⁵/ml, 100 ml/each flask. Assays were carried out with control (no extract), and 10 ml of extract at 10 mg/ml, and 1 mg/ml. Again, toxicity results were used to determine the “high” and “low” concentrations for any given extract. After extract addition, cell cultures were incubated for 24 hours under conditions as outlined above. The cells were counted and subsequently collected in 50 centrifuge tubes. The resulting cell pellet was treated with an RNA isolation means to extract mRNA (see, for example, Sambrook et al., 1989 at pages 7.3-7.39).

Microarray

Microarray printing was carried out as follows:

Human gene clones were obtained from the IMAGE Consortium libraries through its distributors and comprise genes from various tissues. Most clones have been partially sequenced and the sequences were available as expressed sequence tags in the dbEST database of GenBank. Clones were cultured and amplified using commercially available primers prior to application on nylon membranes (Chen et al., Genomics (1998) 51:313-324). Approximately 10 ng of each amplified target was applied on a positively charged nylon membrane using a PC (personal computer) controlled arraying system. The arraying system allows high density spotting and is capable of depositing 31,000 spots on a piece of nylon membrane measuring 18 by 27 mm using a 24-pin flaying tool.

cDNA Probe and Membrane Hybridization

Two microgram of each mRNA sample (mRNA was isolated as outlined above) was labeled with biotin and/or digoxigenin using random primed reverse transcription. The labeled samples were treated, with alkali and the resulting labeled nucleic acids were precipitated prior to use in hybridization. Membrane hybridization and washing were carried out using the labeled probes as disclosed in Chen et al. (1998). To detect the spots on the membrane in dual color mode (i.e., both biotin and digoxigenin), β-galactosidase-conjugated streptavidin (Strept-Gal) and alkaline phosphatase-conjugated digoxigenin antibody (anti-Dig-AP) were employed. After color development, image digitization using an imaging means was employed (e.g., a flatbed scanner or digital camera). Quantitative measurements were determined by computer analysis which uses a program that measures the integrated density of the primary color components of each spot, performs regression analysis of the integrated density data and locates statistical outliers as differentially expressed genes.

Gene Expression Data for Samples 1, 2 and Licorice (ST117)

Extract 1, 2 and 6 corresponding to extract of Cordyceps sinensis, Poria cocos (ST 027) and licorice, respectively, were assayed by the following method: Batches were evaluated for toxicity using Jurkat T cells.

The extracts were prepared as outlined in Example 6. The cells were divided into 24 well culture plates by adding 1 ml of Jurkat cells at a density of 5×10⁵/ml. Assays were carried out with control (no extract), and 5 concentrations of extracts as described (see Table 9). The high and low concentrations for the cell culture assays were varied between 10 mg/ml and 0.05 mg/ml (ie., mg dry weight of herbal extract per ml) depending on the toxicity of the extract to cells. For certain samples the toxicities at 10 mg/ml were such that “high” and “low” concentrations were adjusted downward, nevertheless, at least one order of magnitude between extremes was maintained. For example, for licorice (ST117) the “high” was 0.5 mg/ml and the “low” was 0.05 mg/ml (see Table 9). After extract addition, cell cultures were incubated for 24 hours under conditions as outlined in Example 6. The cells were counted and the resulting data tabulated to demonstrate extract toxicity. The resulting data is shown in Table 9.

TABLE 9
Survival Cell Number at Different
Concentration of Herbal Extract Solution

| | | | | | | | experiment | |
|---|---|---|---|---|---|---|---|---|
| | | | | | | | concentration | |
| | 10 | | | | | | High conc | Low conc. |
| no. × 10⁵ ml | mg/ml | 5 mg/ml | 2 mg/ml | 1 mg/ml | 0.5 mg/ml | No drug | (mg/ml) | (mg/ml) |
| 1 Cordyceps | 8.4 | 11.9 | 11.5 | 9.2 | 9.0 | 12.4 | 10 | 1 |
| sinensis | | | | | | | | |
| mycelium | | | | | | | | |
| 2 ST027 | 4.2 | 8.7 | 10 | 7.5 | 10 | 10 | 10 | 1 |
| 3 ST0-44 | — | 5.9 | 8.4 | 9.9 | 9.4 | | 5 | 0.5 |
| 4 ST051 | — | — | — | 1.7 | 5.4 | | 0.5 | 0.05 |
| 5 ST093 | — | — | 1.9 | 3.8 | 4.4 | | 0.5 | 0.05 |
| 6 ST117 | — | 1.6 | 3.6 | 4.6 | 5.8 | | 0.5 | 0.05 |
| 7 ST123 | 3.4 | 6.4 | 8 | 9.3 | 7.8 | | 5 | 0.5 |
| 8 ST128 | 3.5 | 7.7 | 7.9 | 7.7 | 8.3 | | 5 | 0.5 |
| 9 ST134 | 2.9 | 6.1 | 11.2 | 9.6 | 9.8 | | 5 | 0.5 |
| 10 ST237 | — | — | 2.5 | 6.6 | 8.7 | | 1 | 0.1 |

Note:
original cell number is 5 × 10⁵/ml and the number to 10 × 10⁵/ml after 24 h incubation.
“—” describes all dead cells.

Protocol:

1. Add 1 ml of 5×10⁵/ml Jurkat cells into 24 well culture plates.

2. Prepare 12 kinds of herbal extract solutions and sterilize.

3. Test 5 concentration per sample. 10 mg/ml, 5 mg/ml, 2 mg/ml, 1 mg/ml, 0.5 mg/ml

4. Culture the cells for 24 h in 37C with 5% CO₂ incubator.

In each analysis, 144×96 genes (i.e., 13,824 genes) were analyzed (data not shown) and about 100 genes showed significant differences in comparison with that of control (Table 10). Some of the genes were up-regulated and others were down-regulated. The magnitude of the difference with the control sometimes varied depending on the relative amount of the herbal composition to which the particular cells were exposed. Numbers under C1 (control treatment) and H or L (herbs) represent intensities of mRNA expressed after subtraction of background (Table 10). The gene designation is encoded in Array AD, which can be traced to a specific GenBank clone. The level of expression was determined by H or L divided by C. Only a fraction of 13,824 genes in each herb treated samples showed significant changes, namely, up, down or unchanged (see Table 10).

| | The value show in colume K,L,M,N,O,P represent | | | | | | | | | | | | | | |
|---|---|---|---|---|---|---|---|---|---|---|---|---|---|---|---|
| | the ratio of mRNA express level for #1, #2, #6 herbs treatment | | | | | | (H, L for high conc. And low conc) compared with untreated cell (C1) | | | | | | | | |
| | Title | | | | | | 6H | 6L | | | | | | | |
| rem | TRANSDUCIN- | C1 | 1H | 1L | 2h | 2L | 6H | 6L | 1H | 1L | 2h | 2L | Sg- | Sg- | |
| ArrayAD | LIKE ENHANCER PROTEIN 1 | Sg-Bk | Sg-Bk | Sg-Bk | Sg-Bk | Sg-Bk | Bk | Bk | COMPARE TO CONTROL | | | | | | CloneID |
| <94,030> | Adenylosuccinate lyase | 55.29 | 8.55 | 7.15 | 46.74 | 38.07 | 29.21 | 30.82 | 0.154639 | 0.129318 | 0.845361 | 0.688551 | 0.528305 | 0.557424 | 504540 |
| <91,097> | Neurotrophic tyrosine kinase, | 89.55 | 98.29 | 70.26 | 84.83 | 93.83 | 36.02 | 89.63 | 1.097599 | 0.78459 | 0.947292 | 1.047795 | 0.402233 | 1.000893 | 1013392 |
| | receptor, type 1 | | | | | | | | | | | | | | |
| <89,083> | MHC class I protein HLA-A (HLA-A28,-B40, -Cw3) | 80.68 | 61.91 | 46.13 | 59.86 | 29.64 | 46.16 | 32.35 | 0.767353 | 0.571765 | 0.741943 | 0.367377 | 0.572137 | 0.400967 | 510048 |
| <87,083> | | 85.33 | 50.96 | 37.75 | 53.25 | 25.54 | 37.5 | 49.82 | 0.597211 | 0.4424 | 0.624048 | 0.299309 | 0.43947 | 0.583851 | 1188706 |
| <86,131> | ESTs, Highly similar to HELIX-LOOP-HELIX PROTEIN | 99.48 | 109.46 | 105.62 | 56.68 | 67.54 | 35.65 | 75.78 | 1.100322 | 1.061721 | 0.569763 | 0.67893 | 0.358363 | 0.761761 | 72215 |
| <85,129> | INTEGRAL MEMBRANE PROTEIN E16 | 90.64 | 72.12 | 92.75 | 37.97 | 70.08 | 48.47 | 59.36 | 0.795675 | 1.023279 | 0.41891 | 0.773169 | 0.534753 | 0.654898 | 118548 |
| <85,112> | | 35.23 | 60.82 | 48.87 | 44.04 | 29.71 | 28.25 | 59.33 | 1.72637 | 1.38717 | 1.250071 | 0.843315 | 0.801873 | 1.684076 | 115134 |
| <84,075> | | 89.56 | 77.54 | 77.9 | 66.1 | 59.44 | 43.37 | 42.97 | 0.865788 | 0.869808 | 0.738053 | 0.663689 | 0.484256 | 0.47979 | 172767 |
| <83,129> | | 118.83 | 108.15 | 118.05 | 75.84 | 64.09 | 55.8 | 66.27 | 0.910124 | 0.993436 | 0.638223 | 0.539342 | 0.469578 | 0.557687 | 1184183 |
| <82,082> | Homo sapiens androgen receptor | 60.23 | 47.87 | 34.96 | 37.41 | 9.84 | 44.8 | 46.5 | 0.794787 | 0.580442 | 0.621119 | 0.163374 | 0.743815 | 0.772041 | 118235 |
| | associated protein 24 (A [symbol] | | | | | | | | | | | | | | |
| <82,027> | | 65.11 | 90.01 | 66.74 | 66.3 | 64.44 | 39.3 | 78.37 | 1.38243 | 1.025035 | 1.018277 | 0.98971 | 0.603594 | 1.203655 | 171864 |
| <80,129> | | 96.27 | 99.96 | 109.3 | 81.88 | 87.66 | 48.32 | 53.87 | 1.03833 | 1.135348 | 0.850525 | 0.910564 | 0.501922 | 0.559572 | 1172135 |
| <80,035> | Human small GTP | 44.91 | 46.23 | 25.19 | 78.21 | 40.75 | 37.48 | 42.49 | 1.029392 | 0.5609 | 1.741483 | 0.90737 | 0.834558 | 0.946114 | 1172266 |
| | binding protein Rab7 mRNA, complete | | | | | | | | | | | | | | |
| <74,108> | | 65.2 | 77.25 | 68.09 | 60.2 | 66.21 | 51.52 | 70.19 | 1.184816 | 1.044325 | 0.923313 | 1.015491 | 0.790184 | 1.076534 | 37502 |
| <74,012> | | 34.68 | 60.92 | 57.73 | 24.61 | 48.93 | 42.34 | 65.02 | 1.756632 | 1.664648 | 0.709631 | 1.4109 | 1.220877 | 1.874856 | 45672 |
| <73,132> | Human mRNA for KIAA0078 gene, | 109.86 | 103.38 | 82.35 | 85.97 | 69.15 | 61.91 | 32.44 | 0.941016 | 0.74959 | 0.783511 | 0.629437 | 0.563535 | 0.295285 | 79342 |
| | complete cds | | | | | | | | | | | | | | |
| <72,101> | Human trans-Golgi p230 mRNA, | 46.96 | 25.76 | 18.35 | 10.54 | 17.87 | 1.27 | 17.51 | 0.548552 | 0.390758 | 0.224446 | 0.380537 | 0.027044 | 0.372871 | 563992 |
| | complete cds | | | | | | | | | | | | | | |
| <72,014> | Chlordecone reductase | 4.37 | 15.08 | 13.31 | 42.87 | 22.76 | 20.64 | 30.11 | 3.450801 | 3.045767 | 9.810069 | 5.208238 | 4.723112 | 6.89016 | 21759 |
| <70,101> | | 60.86 | 13.55 | 35.23 | 33.39 | 40.65 | 16.36 | 18.7 | 0.222642 | 0.57887 | 0.548636 | 0.667926 | 0.268814 | 0.307263 | 564469 |
| <69,107> | | 37.92 | 31.41 | 26.12 | 82.82 | 28.64 | 36.95 | 50.55 | 0.828323 | 0.688819 | 2.184072 | 0.755274 | 0.97442 | 1.33307 | 29009 |
| <68,064> | Human mRNA for KIAA0034 gene, | 2.24 | 1.27 | 5.31 | 0.21 | 2.5 | 4.2 | 41.64 | 0.566964 | 2.370536 | 0.09375 | 1.116071 | 1.875 | 18.58929 | 51927 |
| | complete cds | | | | | | | | | | | | | | |
| <62,117> | | 106.71 | 94.67 | 83.98 | 81.39 | 61.45 | 77 | 41.8 | 0.887171 | 0.786993 | 0.762721 | 0.57586 | 0.721582 | 0.391716 | 241351 |
| <62,084> | NA | 1.13 | 0 | 0 | 13.55 | 0.58 | 32.81 | 17.71 | 0 | 0 | 11.99115 | 0.513274 | 29.0354 | 15.67257 | 28012 |
| <62,001> | Heterogeneous nuclear ribonucleoprotein A1 | 97.11 | 71.92 | 73.16 | 42.01 | 78.94 | 83.94 | 96.87 | 0.740603 | 0.753372 | 0.432602 | 0.812893 | 0.864381 | 0.997529 | 236388 |
| <61,118> | NA | 89.52 | 59.97 | 54.11 | 66.86 | 43.05 | 65.65 | 34.92 | 0.669906 | 0.604446 | 0.746872 | 0.480898 | 0.733356 | 0.39008 | 240018 |
| <59,142> | ATPase, Na+/K+ transporting, | 13.03 | 15.48 | 47.75 | 27.71 | 27.86 | 23.22 | 16.58 | 1.188028 | 3.66462 | 2.126631 | 2.138143 | 1.782041 | 1.272448 | 121270 |
| | alpha 1 polypeptide | | | | | | | | | | | | | | |
| <59,135> | | 60.85 | 90.23 | 39.83 | 93.01 | 38.17 | 86.39 | 31.2 | 1.482827 | 0.65456 | 1.528513 | 0.62728 | 1.419721 | 0.512736 | 510863 |
| <58,087> | ESTs, Weakly similar | 77.72 | 46.86 | 76.33 | 64.43 | 22.52 | 56.78 | 63.4 | 0.602934 | 0.982115 | 0.829002 | 0.289758 | 0.730571 | 0.815749 | 645239 |
| | to KIAA0062 [H. sapiens] | | | | | | | | | | | | | | |
| <57,016> | Neuroblastoma RAS | 71.71 | 16.47 | 44.61 | 39.51 | 66.89 | 50.43 | 35.52 | 0.229675 | 0.622089 | 0.550969 | 0.932785 | 0.703249 | 0.495328 | 47526 |
| | viral (v-ras) oncogene homolog | | | | | | | | | | | | | | |
| <56,088> | 3-HYDROXY-3- | 56.77 | 52.66 | 59.53 | 59.6 | 16.89 | 72.87 | 60.28 | 0.927603 | 1.048617 | 1.04985 | 0.297516 | 1.2836 | 1.061828 | 509520 |
| | METHYLGLUTARYL-COENZYME A | | | | | | | | | | | | | | |
| <56,060> | Translation elongation factor 1-alpha-1 | 81.26 | 92.28 | 92.08 | 36.86 | 79.09 | 71.74 | 70.47 | 1.135614 | 1.133153 | 0.453606 | 0.973296 | 0.882845 | 0.867216 | 31027 |
| <55,040> | | 0 | 43.58 | 39.47 | 53.67 | 46.42 | 40 | 65.93 | | | | | | | 328351 |
| <54,134> | CARBONIC ANHYDRASE III | 0 | 0 | 4.38 | 43.47 | 8.23 | 32.61 | 9.74 | | | | | | | 287006 |
| <53,048> | H. sapiens mRNA for | 0.25 | 0 | 0 | 33.47 | 0 | 0 | 0.53 | 0 | 0 | 133.88 | 0 | 0 | 2.12 | 116915 |
| | interferon regulatory factor 3 | | | | | | | | | | | | | | |
| <53,023> | Human mRNA for proteasome | 67.76 | 14.98 | 42.33 | 38.48 | 66.52 | 27.24 | 37.24 | 0.221074 | 0.624705 | 0.567887 | 0.9817 | 0.402007 | 0.549587 | 221285 |
| | subunit HsC7-I, complete c | | | | | | | | | | | | | | |
| <52,135> | | 49.26 | 46.81 | 44.4 | 72.95 | 55.5 | 63.21 | 30.35 | 0.950264 | 0.90134 | 1.480918 | 1.126675 | 1.283191 | 0.616119 | 286222 |
| <51,131> | ATP citrate lyase | 2.02 | 0 | 52.06 | 39.33 | 6.03 | 22.36 | 5.6 | 0 | 25.77228 | 19.4703 | 2.985149 | 11.06931 | 2.772277 | 624420 |
| <51,101> | POLYADENYLATE-BINDING PROTEIN | 8.65 | 0.17 | 0.45 | 38.99 | 14.31 | 38.25 | 11.64 | 0.019653 | 0.052023 | 4.507514 | 1.654335 | 4.421965 | 1.345665 | 529138 |
| <50,035> | SERUM ALBUMIN PRECURSOR | 0.88 | 0 | 10.21 | 44.14 | 0 | 0.79 | 0.01 | 0 | 11.60227 | 50.15909 | 0 | 0.897727 | 0.011364 | 510245 |
| <47,055> | ESTs | 38.53 | 31.69 | 19.45 | 43.8 | 56.1 | 59.36 | 53.2 | 0.822476 | 0.504801 | 1.136777 | 1.456008 | 1.540618 | 1.380742 | 26801 |
| <46,097> | | 34.26 | 35.95 | 32.49 | 39.81 | 63.19 | 30.86 | 51.76 | 1.049329 | 0.948336 | 1.161996 | 1.844425 | 0.900759 | 1.5108 | 511591 |
| <45,100> | Ribosomal protein L5 | 26.15 | 33.6 | 26.3 | 55.86 | 48.59 | 31.26 | 43.29 | 1.284895 | 1.005736 | 2.136138 | 1.858126 | 1.195411 | 1.655449 | 511410 |
| <44,103> | NA | 72.01 | 54.36 | 70.27 | 75.43 | 91.53 | 70.5 | 80.86 | 0.754895 | 0.975837 | 1.047493 | 1.271073 | 0.979031 | 1.1229 | 26099 |
| <43,039> | | 55.86 | 18.02 | 20.83 | 11.68 | 17.8 | 11.82 | 41.06 | 0.322592 | 0.372897 | 0.209094 | 0.318654 | 0.2116 | 0.735052 | 592919 |
| <43,035> | 14-3-3 PROTEIN TAU | 86.86 | 23.18 | 36.23 | 18.14 | 12.76 | 12.99 | 42.28 | 0.266866 | 0.417108 | 0.208842 | 0.146903 | 0.149551 | 0.48676 | 120011 |
| <43,019> | | 72.48 | 10.4 | 23.38 | 27.59 | 12.83 | 3.47 | 15.99 | 0.143488 | 0.322572 | 0.380657 | 0.177014 | 0.047875 | 0.220613 | 488154 |
| <42,099> | | 30.25 | 33.73 | 36.83 | 61 | 51.56 | 40.36 | 48.43 | 1.115041 | 1.217521 | 2.016529 | 1.704463 | 1.334215 | 1.600992 | 86102 |
| <40,100> | ESTs | 0 | 0 | 0.2 | 38.37 | 12.78 | 2.59 | 7.47 | | | | | | | 84335 |
| <39,118> | Thioredoxin | 4.26 | 0.34 | 2.83 | 37.28 | 8.03 | 2.25 | 9.67 | 0.079812 | 0.664319 | 8.751174 | 1.884977 | 0.528169 | 2.269953 | 123764 |
| <39,007> | Pre-alpha (globulin) inhibitor, | 9.46 | 24.06 | 15.82 | 26.08 | 25.48 | 22.8 | 44.36 | 2.54334 | 1.672304 | 2.756871 | 2.693446 | 2.410148 | 4.689218 | 23643 |
| | H3 polypeptide | | | | | | | | | | | | | | |
| <38,097> | NA | 11.81 | 6.94 | 23.82 | 80.14 | 16.65 | 6.13 | 18.51 | 0.587638 | 2.016935 | 6.785775 | 1.409822 | 0.519052 | 1.567316 | 81665 |
| <38,081> | Human GP36b glycoprotein mRNA, | 101.51 | 82.61 | 89.86 | 22.88 | 71.4 | 89.93 | 79.82 | 0.813811 | 0.885233 | 0.225397 | 0.703379 | 0.885923 | 0.786326 | 1185901 |
| | complete cds | | | | | | | | | | | | | | |
| <35,096> | | 47.35 | 24.52 | 0 | 23.2 | 32.88 | 37.19 | 35.29 | 0.517846 | 0 | 0.489968 | 0.694403 | 0.785428 | 0.745301 | 723779 |
| <34,143> | | 61.23 | 2.99 | 25.05 | 30.61 | 16.93 | 27.03 | 40.4 | 0.048832 | 0.409113 | 0.499918 | 0.276498 | 0.44145 | 0.659807 | 712549 |
| <32,118> | ESTs, Weakly similar to | 21.98 | 8.91 | 14.11 | 53.54 | 24.01 | 17.46 | 22.99 | 0.405369 | 0.641947 | 2.435851 | 1.092357 | 0.794359 | 1.045951 | 265480 |
| | CASEIN KINASE I HOMOLOG | | | | | | | | | | | | | | |
| <32,034> | Pyruvate kinase, muscle | 78.41 | 54.15 | 79.36 | 29.56 | 40.5 | 28.52 | 61.59 | 0.690601 | 1.012116 | 0.376993 | 0.516516 | 0.363729 | 0.785487 | 1170289 |
| <30,102> | RETINOBLASTOMA BINDING | 27.3 | 23.1 | 16.06 | 62.35 | 34.39 | 16.52 | 47.49 | 0.846154 | 0.588278 | 2.283883 | 1.259707 | 0.605128 | 1.73956 | 612365 |
| | PROTEIN P48 | | | | | | | | | | | | | | |
| <30,033> | | 70.49 | 30.32 | 65.13 | 17.18 | 33.65 | 49.07 | 36.52 | 0.430132 | 0.923961 | 0.243723 | 0.477373 | 0.696127 | 0.518088 | 82236 |
| <29,058> | NA | 25.77 | 16.66 | 0 | 59.85 | 33.24 | 27.05 | 18.54 | 0.646488 | 0 | 2.322468 | 1.289872 | 1.04967 | 0.719441 | 38798 |
| <29,035> | | 60.19 | 57.12 | 86.66 | 46.47 | 56.59 | 61.01 | 58.47 | 0.948995 | 1.439774 | 0.772055 | 0.940189 | 1.013624 | 0.971424 | 81491 |
| <28,104> | Ribosomal protein S13 | 0 | 0 | 0 | 37.19 | 1.37 | 0 | 5.97 | | | | | | | 595769 |
| <28,035> | | 71.46 | 35.93 | 51.86 | 11.27 | 30.75 | 40.12 | 36.54 | 0.502799 | 0.725721 | 0.157711 | 0.430311 | 0.561433 | 0.511335 | 994662 |
| <27,119> | | 0 | 0 | 0 | 48.27 | 1.66 | 0 | 1.1 | | | | | | | 114662 |
| <27,097> | ESTs, Moderately similar | 16.06 | 24.53 | 9.26 | 76.01 | 24.34 | 24.16 | 30.01 | 1.527397 | 0.576588 | 4.732877 | 1.515567 | 1.504359 | 1.868618 | 68744 |
| | to Etr-3 [H. sapiens] | | | | | | | | | | | | | | |
| <25,126> | ESTs | 42.93 | 22.03 | 29.88 | 67.76 | 40.39 | 24.86 | 30.53 | 0.513161 | 0.696017 | 1.578383 | 0.940834 | 0.579082 | 0.711158 | 427843 |
| <25,106> | | 61.9 | 48.38 | 43.41 | 91.26 | 58.89 | 39.88 | 51.83 | 0.781583 | 0.701292 | 1.474313 | 0.951373 | 0.644265 | 0.837318 | 42714 |
| <24,098> | Human splicesomal protein | 24.26 | 25.43 | 21.8 | 56.18 | 37.28 | 30.59 | 46.77 | 1.048228 | 0.898599 | 2.315746 | 1.536686 | 1.260923 | 1.927865 | 274769 |
| | (SAP 61) mRNA, complete cd | | | | | | | | | | | | | | |
| <24,071> | HEAT SHOCK 70 KD PROTEIN 1 | 63.92 | 58.19 | 61.1 | 50.86 | 66.27 | 80.57 | 68.86 | 0.910357 | 0.955882 | 0.795682 | 1.036765 | 1.260482 | 1.077284 | 255134 |
| <23,126> | | 5.18 | 1.1 | 10.08 | 54.71 | 12.11 | 2.9 | 10.53 | 0.212355 | 1.945946 | 10.56178 | 2.337838 | 0.559846 | 2.032819 | 416946 |
| <23,099> | Ribosomal protein S3A | 5.13 | 1.93 | 1.25 | 38.99 | 14.35 | 4.51 | 18.73 | 0.376218 | 0.243665 | 7.60039 | 2.797271 | 0.879142 | 3.651072 | 298187 |
| <22,013> | EUKARYOTIC INITIATION FACTOR 4B | 61.8 | 49.95 | 41.52 | 22.99 | 49.11 | 42.7 | 20.04 | 0.808252 | 0.671845 | 0.372006 | 0.79466 | 0.690939 | 0.324272 | 51894 |
| <22,006> | 3-HYDROXY-3- | 83.17 | 57.84 | 39.8 | 40.04 | 44.33 | 46.33 | 35.49 | 0.695443 | 0.478538 | 0.481424 | 0.533005 | 0.557052 | 0.426716 | 563866 |
| | METHYLGLUTARYL-COENZYME A | | | | | | | | | | | | | | |
| <20,126> | MITOCHONDRIAL STRESS-70 | 27.02 | 22.19 | 33.86 | 56.87 | 32.97 | 27.24 | 33.86 | 0.821244 | 1.253146 | 2.104737 | 1.220207 | 1.008142 | 1.253146 | 382766 |
| | PROTEIN PRECURSO | | | | | | | | | | | | | | |
| <20,104> | Human eukaryotic translation | 22.09 | 15.17 | 17.33 | 74.98 | 19.17 | 8.3 | 35.84 | 0.686736 | 0.784518 | 3.394296 | 0.867813 | 0.375736 | 1.622454 | 562992 |
| | initiation factor (eIF3) mRN | | | | | | | | | | | | | | |
| <20,103> | Laminin receptor (2H5 epitope) | 1.45 | 1.26 | 0 | 39.66 | 8.67 | 0.73 | 9.03 | 0.868966 | 0 | 27.35172 | 5.97931 | 0.503448 | 6.227586 | 562928 |
| <19,098> | Human semaphorin (CD100) mRNA, | 17.79 | 14.38 | 10.79 | 52.94 | 28.97 | 12.5 | 31.25 | 0.808319 | 0.606521 | 2.975829 | 1.628443 | 0.702642 | 1.756605 | 248687 |
| | complete cds | | | | | | | | | | | | | | |
| <18,103> | Ribosomal protein L5 | 31.47 | 21.13 | 30.07 | 78.6 | 27.18 | 27.47 | 43.29 | 0.671433 | 0.955513 | 2.497617 | 0.86368 | 0.872895 | 1.375596 | 549382 |
| <18,084> | ESTs | 54.61 | 75.17 | 46.16 | 79.5 | 60.01 | 74.37 | 56.43 | 1.376488 | 0.845266 | 1.455777 | 1.098883 | 1.361838 | 1.033327 | 512336 |
| <17,103> | NA | 0 | 0 | 0 | 33.96 | 2.87 | 3.64 | 9.87 | | | | | | | 549224 |
| <17,097> | NA | 1.52 | 2.1 | 1.21 | 37.77 | 11.59 | 3.43 | 14.35 | 1.381579 | 0.796053 | 24.84868 | 7.625 | 2.256579 | 9.440789 | 327474 |
| <17,062> | | 92.72 | 73.27 | 103.64 | 69.79 | 69.35 | 68.85 | 83.14 | 0.790229 | 1.117774 | 0.752696 | 0.747951 | 0.742558 | 0.896678 | 50753 |
| <16,103> | Integrin, beta 1 (fibronectin | 0 | 0.92 | 0 | 41.3 | 1.84 | 6.67 | 11.18 | | | | | | | 547624 |
| | receptor, beta polypeptide, an | | | | | | | | | | | | | | |
| <16,100> | Homo sapiens E1B | 0.79 | 0 | 0.7 | 38.06 | 8.28 | 0.89 | 16.63 | 0 | 0.886076 | 48.17722 | 10.48101 | 1.126582 | 21.05063 | 308754 |
| | 19K/Bcl-2-binding protein Nip3 mRN | | | | | | | | | | | | | | |
| <15,112> | | 16.47 | 11.65 | 11.87 | 46.52 | 22.98 | 28.22 | 22.43 | 0.707347 | 0.720704 | 2.824529 | 1.395264 | 1.713418 | 1.36187 | 49967 |
| <14,103> | NA | 27.61 | 34.26 | 30.18 | 55.67 | 38.45 | 35.52 | 50.43 | 1.240855 | 1.093082 | 2.016298 | 1.392611 | 1.28649 | 1.826512 | 546545 |
| <14,011> | NA | 47.42 | 34.87 | 68.28 | 38.71 | 53.6 | 50.03 | 52.24 | 0.735344 | 1.439899 | 0.816322 | 1.130325 | 1.05504 | 1.101645 | 32672 |
| <13,114> | Homo sapiens clone 24689 mRNA sequence | 20.64 | 13.87 | 19.2 | 51.26 | 32.34 | 20.27 | 27.7 | 0.671996 | 0.930233 | 2.483527 | 1.56686 | 0.982074 | 1.342054 | 144001 |
| <12,144> | Homo sapiens NADH: ubiquinone | 76.78 | 67.2 | 76.11 | 64.5 | 50.04 | 53.7 | 6.92 | 0.875228 | 0.991274 | 0.840063 | 0.651732 | 0.699401 | 0.090128 | 325580 |
| | oxidoreductase NDUFS | | | | | | | | | | | | | | |
| <12,111> | | 40.04 | 55.04 | 46.34 | 77.78 | 46.07 | 69.51 | 55.69 | 1.374625 | 1.157343 | 1.942557 | 1.150599 | 1.736014 | 1.390859 | 49373 |
| <11,135> | | 90.34 | 114.29 | 87.88 | 80.49 | 87.22 | 60.79 | 87.28 | 1.26511 | 0.97277 | 0.890967 | 0.965464 | 0.672902 | 0.966128 | 510620 |
| <11,105> | Ribosomal protein L3 | 8.27 | 14.41 | 15.59 | 36.82 | 25.87 | 31.97 | 47.1 | 1.742443 | 1.885127 | 4.452237 | 3.128174 | 3.86578 | 5.695284 | 31866 |
| <11,053> | Calpain, large polypeptide L2 | 10.48 | 23.58 | 14.8 | 15.69 | 8.87 | 47.63 | 28.91 | 2.25 | 1.412214 | 1.497137 | 0.846374 | 4.544847 | 2.758588 | 544957 |
| <10,136> | Malic enzyme 2, mitochondrial | 74.34 | 81.63 | 90.94 | 83.18 | 74.85 | 54.35 | 65.41 | 1.098063 | 1.223298 | 1.118913 | 1.00686 | 0.7311 | 0.879876 | 510323 |
| <10,101> | ESTs | 13.65 | 16.34 | 13.97 | 47.57 | 26.68 | 36.05 | 40.14 | 1.19707 | 1.023443 | 3.484982 | 1.954579 | 2.641026 | 2.940659 | 613442 |
| <09,136> | NA | 59.82 | 95 | 87.16 | 77.34 | 83.51 | 69.24 | 71.68 | 1.588098 | 1.457038 | 1.292879 | 1.396021 | 1.157472 | 1.198261 | 509836 |
| <09,110> | ER LUMEN PROTEIN | 17.3 | 41.73 | 48.92 | 55.32 | 35.56 | 45.73 | 60.03 | 2.412139 | 2.827746 | 3.197688 | 2.055491 | 2.643353 | 3.469942 | 47800 |
| | RETAINING RECEPTOR 1 | | | | | | | | | | | | | | |
| <09,085> | Human phospholipid transfer | 80.14 | 88.83 | 96.22 | 80.4 | 77.44 | 76.05 | 82.81 | 1.108435 | 1.200649 | 1.003244 | 0.966309 | 0.948964 | 1.033317 | 613511 |
| | protein mRNA, complete cds | | | | | | | | | | | | | | |
| <08,138> | | 41.57 | 60.4 | 69.53 | 73.27 | 81.26 | 59.49 | 66.73 | 1.452971 | 1.6726 | 1.762569 | 1.954775 | 1.43108 | 1.605244 | 632480 |
| <08,132> | | 67.22 | 102.84 | 103.65 | 90.56 | 88.43 | 76.85 | 81.15 | 1.529902 | 1.541952 | 1.347218 | 1.315531 | 1.143261 | 1.20723 | 66427 |
| <08,122> | Homo sapiens mRNA for | 32.01 | 23.21 | 47.74 | 63.02 | 35.64 | 32.48 | 46.85 | 0.725086 | 1.491409 | 1.96876 | 1.113402 | 1.014683 | 1.463605 | 308580 |
| | DRAK1, complete cds | | | | | | | | | | | | | | |
| <08,105> | | 69.58 | 58.7 | 84.49 | 90.5 | 68.91 | 89.98 | 94.96 | 0.843633 | 1.214286 | 1.300661 | 0.990371 | 1.293188 | 1.36476 | 30607 |
| <08,055> | | 19.5 | 20.01 | 22.83 | 27.87 | 34.66 | 48.86 | 50.43 | 1.026154 | 1.170769 | 1.429231 | 1.777436 | 2.505641 | 2.586154 | 611114 |
| <07,111> | | 30.8 | 44.67 | 48.75 | 73.84 | 51.31 | 59.76 | 68.29 | 1.450325 | 1.582792 | 2.397403 | 1.665909 | 1.94026 | 2.217208 | 46880 |
| <07,107> | ESTs | 4.57 | 3.23 | 9.01 | 41.32 | 8.83 | 22.9 | 23.56 | 0.706783 | 1.971554 | 9.041575 | 1.932166 | 5.010941 | 5.155361 | 29970 |
| <06,135> | | 71.42 | 112.68 | 128.81 | 102.8 | 101.48 | 93.72 | 94.2 | 1.577709 | 1.803556 | 1.439373 | 1.420891 | 1.312237 | 1.318958 | 286790 |
| <06,102> | ESTs | 39.82 | 36.03 | 32.57 | 67.74 | 46.04 | 59.07 | 57.13 | 0.904822 | 0.817931 | 1.701155 | 1.156203 | 1.483425 | 1.434706 | 530700 |
| <06,101> | Intercellular adhesion molecule 2 | 4.43 | 2.95 | 4.21 | 38.95 | 16.6 | 15.85 | 32.59 | 0.665914 | 0.950339 | 8.792325 | 3.747178 | 3.577878 | 7.356659 | 530665 |
| <06,034> | Ribosomal protein L5 | 0 | 0 | 0 | 0 | 33.93 | 0 | 0 | | | | | | | 666094 |
| <06,006> | Human fragile X mental | 0 | 31.71 | 0 | 0 | 0 | 0 | 0 | | | | | | | 530672 |
| | retardation syndrome related protein | | | | | | | | | | | | | | |
| <04,134> | | 39.03 | 54.62 | 46.61 | 71.8 | 58.82 | 64.83 | 64.43 | 1.399436 | 1.19421 | 1.839611 | 1.507046 | 1.66103 | 1.650781 | 285979 |
| <04,106> | Ferritin, light polypeptide | 15.74 | 23.81 | 21.94 | 46.74 | 21.94 | 56.63 | 55.12 | 1.512706 | 1.393901 | 2.969504 | 1.393901 | 3.59784 | 3.501906 | 31309 |
| <03,140> | | 56.08 | 88.33 | 61.29 | 93.1 | 74.04 | 74.42 | 81.16 | 1.575071 | 1.092903 | 1.660128 | 1.320257 | 1.327033 | 1.447218 | 624595 |
| <03,099> | ESTs | 22.97 | 18.57 | 12.1 | 52.83 | 23.48 | 53.26 | 41.55 | 0.808446 | 0.526774 | 2.299956 | 1.022203 | 2.318677 | 1.808881 | 42281 |
| <02,111> | ESTs, Highly similar to | 34.89 | 23.63 | 25 | 77.41 | 28.84 | 56.6 | 60.36 | 0.677271 | 0.716538 | 2.218687 | 0.826598 | 1.622241 | 1.730009 | 45327 |
| | HYPOTHETICAL 64.5 KD PRO | | | | | | | | | | | | | | |
| <01,136> | Homo sapiens mRNA for | 21.36 | 34.84 | 23.95 | 53.14 | 45.66 | 40.67 | 51.53 | 1.631086 | 1.121255 | 2.487828 | 2.13764 | 1.904026 | 2.412453 | 567287 |
| | 5-aminoimidazole-4-carboxami [symbol] | | | | | | | | | | | | | | |
| <01,058> | Acyl-Coenzyme | 60.68 | 53.35 | 73.26 | 62.56 | 56.58 | 72.81 | 91.71 | 0.879202 | 1.207317 | 1.030982 | 0.932432 | 1.199901 | 1.511371 | 30921 |
| Total 116 | A dehydrogenase, C-4 to C-12 straight ch [symbol] | | | | | | | | | | | | | | |

In this manner, we are able to correlate specific gene expression with the exposure of a cell to no, low (L) or high (H) amounts of an herbal composition. Many of the genes identified in this way code for proteins important in known metabolic or biochemical pathways. Many of these proteins have direct and indirect effects on certain physiological, morphological and psychological parameters. Thus, this method permits the association of a particular genetic fingerprint of an herbal composition with its array biological effects. Such associations can be used to profile or characterize an herbal composition for the purposes of Quality Control and Quality Assurance and evaluating pharmacological or toxicological properties. The role of primary and secondary herbs in an herbal formula can also be assessed by this approach.

HPLC Analysis

The herbal batches were analyzed by HPLC with a Beckman ODS Ultrasphere™ column (5 micron particles, 4.6 mm×25 cm) and detected with an UV spectrophotometer (Perkin Elmer). The wavelengths for UV detection were monitored at 280 nm and 340 nm. The mobile phase was pumped at 1 ml/min and consisted of Solvent A: H₂O and Solvent B: 20% MeOH with the following gradient: 1) the solvent was 100% solvent A for the first 5 minutes; 2) the solvent composition was changed to 10% solvent A/90% solvent B for the next 10 minutes; and 3) the solvent was changed to 10% solvent A/90% solvent B for the next 40 minutes. This was followed by the addition of 100 % solvent A for 5 minutes. The HPLC marker is glycyrrhizin.

Algorithm

The data collected form part of the multidimensional analysis used to generate multivariant normal distribution sets as a means of determining a baseline correlation between biological activity and standard licorice molecular, chemical (HPLC and Mass Spec), and origin/growth characteristics.

B. Scute

Evaluation of Radix Scutellariae (Scute)

Scute has been found to be useful in reducing capillary permeability and inflammation. It can also be used treat enteritis and dysentery, increases the secretion of bile to treat jaundice; to relieve muscle spasms, to treat coughing and to expel parasites. The properties of the scute batches used in this example are presented in Table 11.

TABLE 11
Batch Properties (Scute)

| Property | Batch A | Batch B | Batch C | Batch D |
|---|---|---|---|---|
| Plant Name | Scutellariae | Scutellariae | Scutellariae | Scutellariae |
| | radix | radix | radix | radix |
| Origin | Sanxi | U.S., Kin | U.S., Kin | U.S., Boston |
| | Province. | Man Herb | Man Herb | |
| | | Center | Center | |
| Preparation | Standard | Boiled, | Warm H₂O, | Boiled, 2 hours |
| method | | 30 min | 30 min. | |
| Plant part | Root | — | — | — |

Biological and Enzyme Assays

Briefly, one gram of each preparation of scute extract was added with 10 ml of water (1 mg/ml). The mixture was treated as Outlined in Table 11. The supernatant was collected after centrifugation and filtered through a 0.22 μm filter. Batches of scute were tested against either HepG2 cells (ATCC cat #HB8065) or Jurkat T cells (ATCC cat #TIB-152) or both. One to fifty dilutions were used for each assay. Cells were cultured for 24 hours as described above.

Batches were also evaluated for the ability to inhibit hepatitis B virus as detected by DNA quantitation (see Dong et al. Proc Natl Acad Sci USA (1991) 88: 8495-8499). Briefly, one gram of preparation was added with 10 ml of water. The mixture was treated as outlined in Table 11. The supernatant was collected after centrifugation and filtered through a 0.22 μm filter.2.2.15 cells which secrete hepatitis B virons (kindly provided by Professor G. Ace; see Ace et al. Proc Natl Acad Sci USA (1987) 84: 1005-1009) were used in this assay. One to fifty dilutions were used for each assay. The cell growth inhibition assay was performed for 72 hours. All other procedures were performed as described by Dong et al., Proc Natl Acad Sci USA (1991) 88: 8495-8499.

For β-glucuronidase, different scute extracts were added to triplicate wells of a 96-well plate which contained 0.1 mM phenolphthalein glucuronidate, 70 mM Tris-HCl (pH 6.8) and 0.8 ng of dialyzed β-glucuronidase (from E. Coli, purchased from Sigma) to a final volume of 80 μl. After 2 hr incubation at 37° C., the reactions were terminated with 200 μl of stopping solution which contained 0.2 M Glycine and 0.2 M NaCl (pH 10.4), and the OD was monitored with a kinetic microplate reader at 540 nm.

The results of the assays using the three batches is displayed in Table 12.

TABLE 12
Biological Assay of Four Preparations of Scute*

| | E. Coli | | | |
|---|---|---|---|---|
| | β-Glucuronidase | HepG2 | Jurkat | HBV‡ DNA |
| Scute A | 1.5 | 0.33 | 0.45 | None |
| Scute B | 1.8 | ND | ND | ND |
| Scute C | 0.3 | ND | ND | ND |
| Scute D | ND | 0.65 | ND | 27.5 |

*Values represent IC₅₀ values.
‡% of Control
ND, not determined.

Evaluation of Scute Effects on Protein Expression

HepG2 cells (1×10⁶) were seeded in 25 cm² flasks in 3.0 ml of RPMI-1640 medium (see Life Technologies, Inc., Catalogue and Reference Guide, 1998-1999, Cell Culture section) 24 hr before the extract addition. The cells were treated with or without herbal medicine, where the former is added at two final concentrations of 0.2 mg/ml or 4 mg/ml, respectively, and incubated at 37° C. for 24 hours. The medium was removed and the cells were washed twice with cold PBS. The cells were harvested into 1 ml of PBS and centrifuged at 10,000 rpm for 2 minutes, extracted on ice with a buffer containing 50 MM Tris-Cl (pH 7.5), 0.2 mM PMSF and 10% glycerol, followed by three freeze-thaw cycles. Potassium chloride was added to the cell lysate at a final concentration of 0.15 M prior to centrifugation. The protein concentration was determined and the cell extract was electrophoresed according to the method of Laemmli U. K. (Nature (1970) 227: 680-685); Western blots were performed by standard techniques known in the art, see for example Sambrook, et al (1989). The antibodies used were directed to the following proteins: Topo I, Stat (20707); Cyclin B1; MAPK (Ab2) and Nm 23 H1.

FIG. 4 demonstrates that scute batches A and B do not differentially affect the expression of the polypeptides-resolved on Western blots.

HPLC Analysis

The herbal batches were analyzed by HPLC with a Beckman ODS Ultrasphere™ column (5 micron particles, 4.6 mm×25 cm) and detected with an UV spectrophotometer (Perkin Elmer). The wavelengths for UV detection were monitored at 280 nm and 340 nm. The mobile phase was pumped at 1 ml/min and consisted of Solvent A: H₂O and Solvent B: 20% MeOH with the follow gradient: 1)the solvent was 100% solvent A for the first 5 minutes; 2) the solvent composition was changed to 10% solvent A/90% solvent B for the next 10 minutes; and 3) the solvent was changed to 10% solvent A/90% solvent B for the next 40 minutes. This was followed by the addition of 100 % solvent A for 5 minutes. The HPLC markers are baicalin and baicalein.

Scute batches in water and acid treated samples were analyzed by HPLC. Water and acid treated batches were virtually indistinguishable.

Algorithm

The data collected form part of the multidimensional analysis used to generate multivariant normal distribution sets as a means of determining a baseline correlation between biological activity and standard scute chemical (HPLC), and origin/growth characteristics.

C. White Peony Root

Evaluation of Paeonie lactiflora pallus radix (Peony)

Peony is used to suppress and soothe pain. It is also known to soothe ligaments and purify the blood. The properties of the peony batches used in this example are presented in Table 13.

TABLE 13
Batch Properties (Peony)

| Property | Batch A | Batch B |
|---|---|---|
| Plant Name | Paeonie lactiflora pallus | Paeonie lactiflora pallus |
| Origin | Anwey Province | U.S., Boston |
| Preparation | Standard | Boiled 2 hours. |
| method | | |
| Plant part | Root | Root |

Biological and Enzyme Assays

Briefly, one gram of each preparation of scute extract was added with 10 ml of water (1 mg/ml). The mixture was treated as outlined in Table 13. The supernatant was collected after centrifugation and filtered through a 0.22 μm filter. Batches of peony were tested against either HepG2 cells (ATCC cat #HB-8065) or Jurkat T cells (ATCC cat #TIB-152) or both. One to fifty dilutions were used for each assay. Cells were cultured for 24 hours as described above.

Batches were also evaluated for the ability to inhibit hepatitis B virus as detected by DNA quantitation (see Dong et al., Proc Natl Acad Sci USA (1991) 88: 8495-8499).

Briefly, one gram of preparation was added with 10 ml of water. The mixture was treated as outlined in Table 13. The supernatant was collected after centrifugation and filtered through a 0.22 μm filter. 2.2.15 cells which secrete hepatitis B virons (kindly provided by Professor G. Ace; see Ace et al. Proc Natl Acad Sci USA (1987) 84: 1005-1009) were used in this assay. One to fifty dilutions were used for each assay. The cell growth inhibition assay was performed for 72 hours. All other procedures were performed as described by Dong et al., Proc Natl Acad Sci USA (1991) 88: 8495-8499.

Different peony extracts were added to triplicate wells of a 96-well plate which contained 0.1 mM phenolphthalein glucuronidate, 70 mM Tris-HCl (pH 6.8) and 0.8 ng of dialyzed beta-glucuronidase (from E. coli, purchased from Sigma) to a final volume of 80 μl. After 2 hr incubation at 37° C., the reactions were terminated with 200 μl of stopping solution which contained 0.2 M Glycine and 0.2 M NaCl (pH 10.4), and the OD was monitored with a kinetic microplate reader at 540 nm. Results are shown in Table 14.

TABLE 14
Biological Assay of Two Preparations of Peony*

| | E. Coli | | | |
|---|---|---|---|---|
| | β-Glucuronidase | HepG2 | Jurkat | HBV‡ |
| Peony A | 2.8 | >1.5 | 1.1 | None |
| Peony B | >2.5 | ND | ND | ND |

*Values represent IC₅₀ values.
‡% of Control
ND, not determined.

HPLC Analysis

The herbal batches were analyzed by HPLC with a Beckman ODS Ultrasphere column (5 micron particles, 4.6 mm×25 cm) and detected with an UV spectrophotometer (Perkin Elmer). The wavelengths for UV detection were monitored at 280 nm and 340 nm. The mobile phase was pumped at 1 ml/min and consisted of Solvent A: H₂O and Solvent B: 20% MeOH with the following gradient: :1) the solvent was 100% solvent A for the first 5 minutes; 2) the solvent composition was changed to 10% solvent A/90% solvent B for the next 10 minutes; and 3) the solvent was changed to 10% solvent A/90% solvent B for the next 40 minutes. This was followed by the addition of 100% solvent A for 5 minutes. HPLC marker is paeoniflorin.

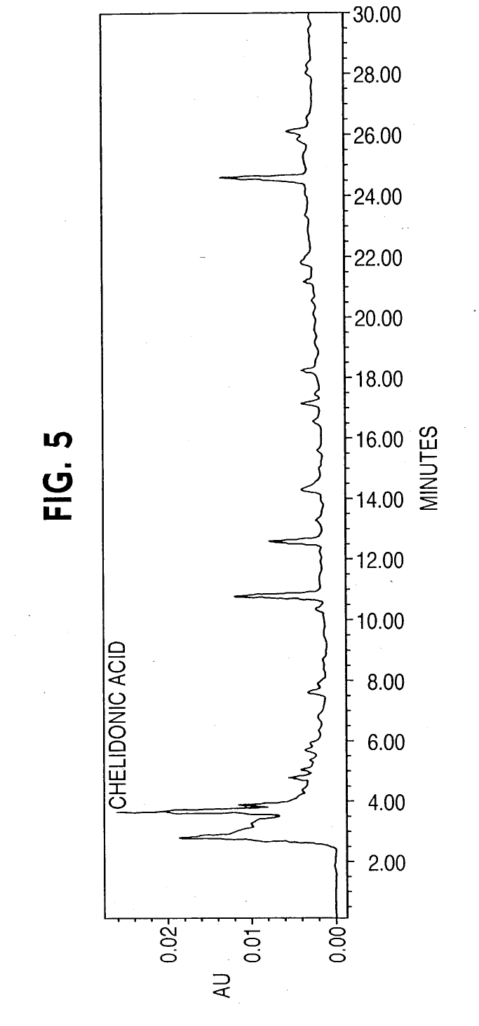

Peony batches were analyzed by HPLC as shown in FIG. 5.

Algorithm

The data collected form part of the multidimensional analysis used to generate multivariant normal distribution sets as a means of determining a baseline correlation between biological activity and standard peony chemical (HPLC), and origin/growth characteristics.

D. Date

Evaluation of Ziziphi Fructus (Date)

Date has been used for diuretic properties and strengthening effects. The properties of the date batches used in this example are presented in Table 15.

TABLE 15
Batch Properties (Date)

| Property | Batch A | Batch B | Batch C |
|---|---|---|---|
| Plant Name | Ziziphi Fructus | Ziziphi Fructus | Ziziphi Fructus |
| Origin | Hebei | U.S., Kin Man Herb | U.S., Kin Man Herb |
| | Province. | Center | Center |
| Preparation | Standard | Boiled, 30 min | Warm H₂O, 30 min. |
| method | | | |
| Plant part | Fruit | — | — |

Biological and Enzyme Assays

Briefly, one gram of each batch of scute extract was added with 10 ml of water (1 mg/ml). The mixture was treated as outlined in Table 15. The supernatant was collected after centrifugation and filtered through a 0.22 μm filter. Batches of date were tested against either HepG2 cells (ATCC cat #HB-8065) or Jurkat T cells (ATCC cat #TIB-152) or both. One to fifty dilutions were used for each assay. Cells were cultured for 24 hours as described above.

Batches were also evaluated for the ability to inhibit hepatitis B virus as detected by DNA quantitation (see Dong et al., Proc Natl Acad Sci USA (1991) 88: 8495-8499). Briefly, one gram of preparation was added with 10 ml of water. The mixture was treated as outlined in Table 15. The supernatant was collected after centrifugation and filtered through a 0.22 μm filter. HepG2.2.15 cells which secrete hepatitis B virons (kindly provided by Professor G. Ace; see Ace et al. Proc Natl Acad Sci USA (1987) 84: 1005-1009) were used in this assay. One to fifty dilutions were used for each assay. The cell growth inhibition assay was performed for 72 hours. All other procedures were performed as described by Dong et al., Proc Natl Acad Sci USA (1991) 88: 8495-8499.

Different peony extracts were added to triplicate wells of a 96-well plate which contained 0.1 mM phenolphthalein glucuronidate, 70 mM Tris-HCl (pH 6.8) and 0.8 ng of dialyzed beta-glucuronidase (from E. coli, purchased from Sigma) to a final volume of 80 μl. After 2 hr incubation at 37° C., the reactions were terminated with 200 μl of stopping solution which contained 0.2 M Glycine and 0.2 M NaCl (pH 10.4), and the OD was monitored with a kinetic microplate reader at 540nm. Results are shown in Table 16.

TABLE 16
Biological Assay of Three Preparations of Date*

| | E. Coli | | | |
|---|---|---|---|---|
| | β-Glucuronidase | HepG2 | Jurkat | HBV‡ DNA |
| Date A | 1.2 | 1.5 | 5.1 | None |
| Date B | ND | >2.0 | ND | 52.3 |
| Date C | 2.5 | ND | ND | ND |

*Values represent IC₅₀ values.
‡% of Control
ND, not determined.

HPLC Analysis

The herbal batches were analyzed by HPLC with a Beckman ODS Ultrasphere column (5 micron particles, 4.6 mm×25 cm) and detected with an UV spectrophotometer (Perkin Elmer). The wavelengths for UV detection were monitored at 280 nm and 340 nm. The mobile phase was pumped at 1 ml/min and consist of Solvent A: H₂O and Solvent B: 20% MeOH with the following gradient: 1) the solvent was 100% solvent A for the first 5 minutes; 2) the solvent composition was changed to 10% solvent A/90% solvent B for the next 10 minutes; and 3) the solvent was changed to 10% solvent A/90% solvent B for the next 40 minutes. This was followed by the addtion of 100 % solvent A for 5 minutes. HPLC markers for date are chelidonic acid and cAMP.

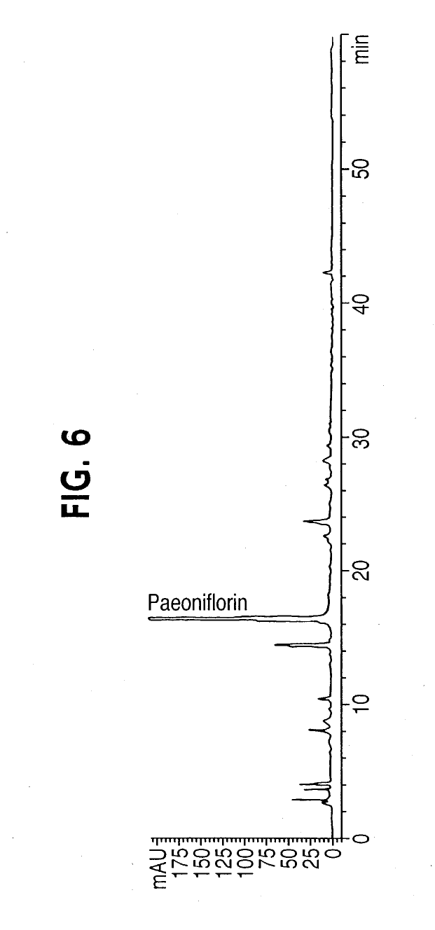

Date batches samples were analyzed by HPLC as shown in FIG. 6.

Algorithm

The data collected form part of the multidimensional analysis used to generate multivariant normal distribution sets as a means of determining a baseline correlation between biological activity and standard peal (HPLC), and origin/growth characteristics.

### Example 15

Characterizing Herbal Medicines by Nucleic Acid Microarray Analysis.

Introduction.

The rapid development of nucleic acid microarray technology has led to an explosion of gene expression data (Lander, 1999, Duggan et al., 1999). Four characteristics of the gene expression account for the great value of using nucleic acid microarrays to study the gene expression profiles. (i) Nucleic acid microarray makes it easier to measure the transcripts of thousands of genes at once. (ii) Close association between the function of a gene product and its expression pattern makes gene function predictable. (iii) Cells respond to the micro-environmental changes by changing the expression level of specific genes. (iv) The sets of genes expressed in a cell determine what the cell is derived of, what biochemical and regulatory systems are involved and so on (Brown and Botstein, 1999). By using a microarray system, the above features can be studied in an ensemble manner.

The expression of any desired number of genes can be detected using the nucleic acid microarray technology. For example, up to about 20,000 genes may be placed on a single array. We have developed a nucleic acid microarray with colorimetric detection system (Microarray/CD) (Chen et al., 1998). Gene expression profiles of different cell lines were studied using microarray filter membranes (2.7 cm×1.8 cm) with about 10,000 cDNA representing approximately 10,000 distinct human transcripts. The sensitivity and detection limits of the microarray/CD system have been characterized and are comparable to the system with radioactive detection or the system with laser induced fluorescence detection (Bertucci et al., 1999).

As previously described, cellular gene expression profiles portray the origin, the present differentiation of the cell, and the cellular responses to external stimulants. In other words, the gene expression profiles reveal the state of the cell and microarray is a perfect tool for the rendering purpose. In the present studies, we apply the microarray/CD system to characterize cellular responses to external stimulants, in this case, the Chinese herbal medicines. Conversely, we also based on the stimulated gene expression profiles to classify different herbal medicines.

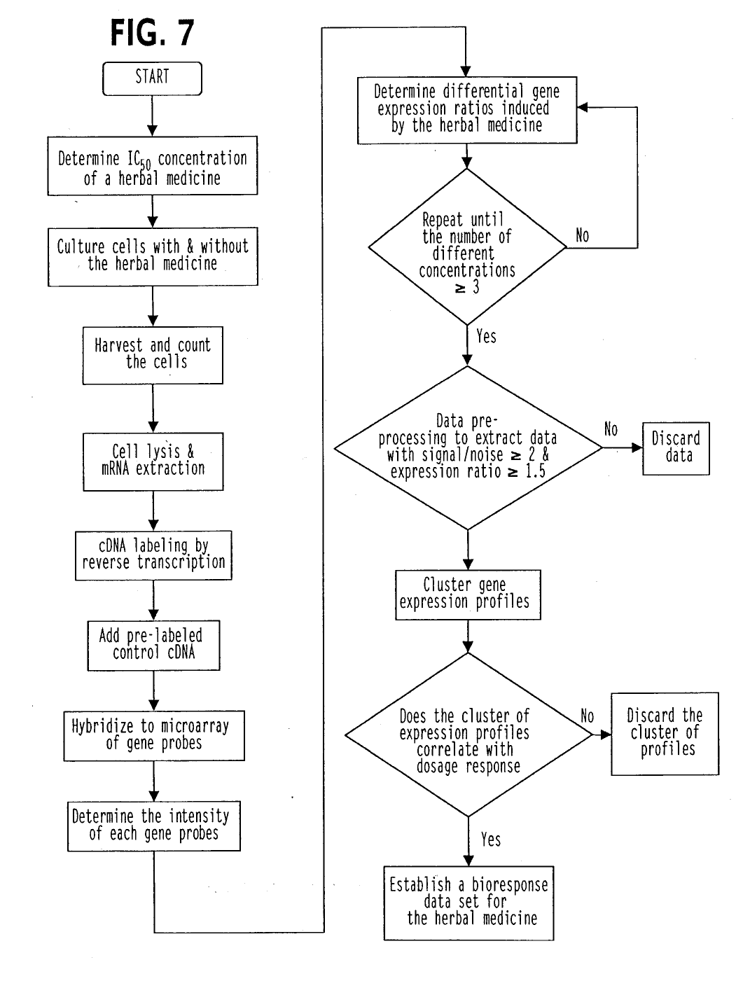

FIG. 7 is a flowchart depicting a general method that may be used for establishing an expression response data set for cells treated with an herbal composition. The method comprises the steps of:

(a) Determine the IC₅₀ concentration of an herbal composition by incubating various concentrations of the herbal medicine in mammalian cell cultures and identify the concentration that leaves 50% of survival cells after a predetermined time.

(b) Incubate the mammalian cell cultures with herbal extracts of various fractions of IC₅₀ concentrations.

(c) Harvest and count the cultured cells after a predetermined culture time.

(d) Immediately lyse the cells after they are removed from the incubator and extract mRNA from cell lysate.

(e) Label the mRNA by reverse transcription reaction to turn mRNA into labeled cDNA.

(f) Mix the labeled cDNA with control cDNA of plant origin and perform hybridization to a microarray of mammalian gene probes.

(g) Measure expression level of genes by analyzing digitized images of the microarray hybridization results.

(h) Perform data pre-processing to select data for statistical analysis.

(i) Acquired expression data generated by microarray experiments of an herbal composition with various concentrations.

(j) Data pre-processing to select the genes with statistical significance in cells treated with different concentrations of the herbal medicine.

(k) Categorize expression profiles into clusters by statistical methods such as the self-organizing-map algorithm.

(l) Deduce the characteristic expression profiles for the herbal medicine based on the expression profile clusters.

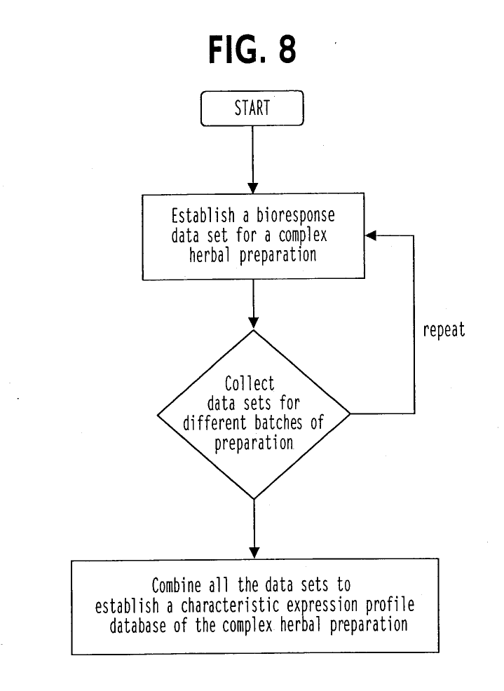

FIG. 8 is a flowchart demonstrating how data sets of expression data for various batches of the herbal composition are integrated to make an expression profile database for the particular herbal composition. The expression profile database then becomes part of the HBR Array.

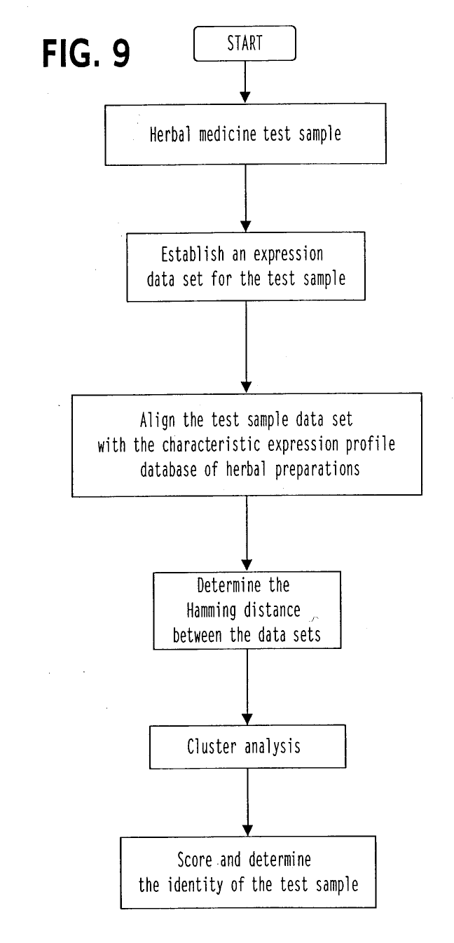

HBR Arrays containing expression profiles may also be used to identify an unknown herbal composition. FIG. 9 is a flowchart depicting a general method for identifying an unknown herbal composition the method comprising the steps of:

(a) Construct an HBR Array containing characteristic expression profiles for an herbal medicine or a collection of expression profiles of various herbal medicines by the aforementioned steps.

(b) Obtain the characteristic expression profile data set of the unknown herbal composition.

(c) Compare the HBR Array containing the characteristic expression profile induced by the said unknown herbal composition with a standardized HBR Array containing expression data by algorithms such as the Hamming distance algorithm.

(d) Score possible alignments to identify the most probable herbal composition whose characteristic expression profiles are archived in the said HBR Array.

Scoring possible alignments of HBR Arrays containing expression profiles may be performed using hierarchical cluster analysis of the Hamming distance matrix. Use of hierarchical cluster analysis for the Hamming distance matrix is well known in the art.

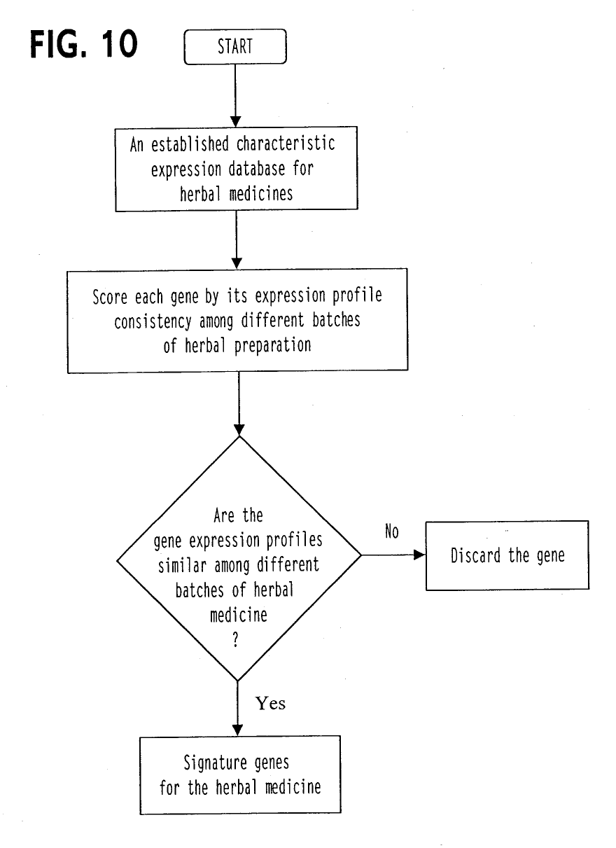

The gene expression profiles may also be incorporated into the standardized HBR Array. As has been already discussed, the standardized HBR Array containing such gene expression profiles induced by an herbal composition can be used for studying the pharmacological mechanisms of the herbal composition, for discovering new application of the herbal composition, and for designing optimized formulation of a complex herbal preparation. As can be seen from the flowchart of FIG. 10, the method may be generally outlined as comprising the steps of:

(a) Construct a data set containing the characteristic gene expression profiles for an herbal composition.

(b) Score each gene by the consistency of its expression profiles in the data set using known statistical parameters, such as the coefficient of variation.

(c) Based on the statistical scoring, gene expression profiles for an herbal medicine are selected to be incorporated into the standardized HBR Array.

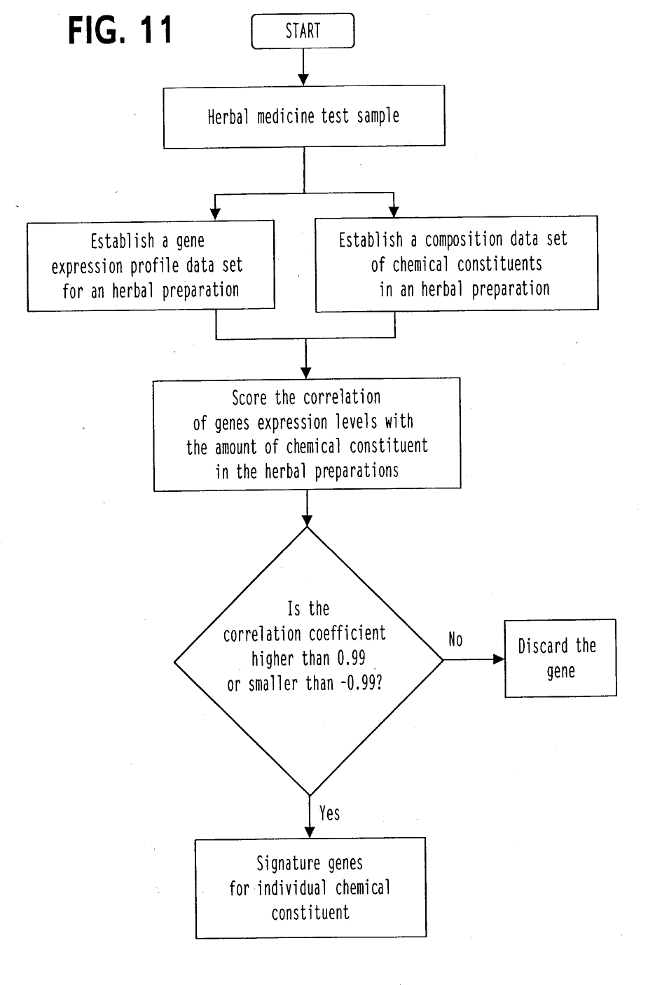

HBR Arrays containing gene expression profiles may also be used to identify signature gene expression profiles induced by individual chemical constituents in an herbal composition consisting of complex chemical constituents, as outlined in the flowchart of FIG. 11. The method comprises the steps of:

(a) Construct a HBR Array containing characteristic gene expression profiles for an herbal composition by the aforementioned steps.

(b) Determine the composition of chemical constituents in an herbal medicine by high performance liquid chromatography (HPLC) or liquid chromatography mass spectrometry (LC-MASS).

(c) Repeat the step (b) for various batches of herbal medicine preparations.

(d) Score the correlation coefficients between the expression levels of each gene with the amount of individual chemical constituent in an herbal preparation.

(e) The signature gene expression profiles for individual chemical constituent are selected with a Pearson correlation coefficient exceeding 0.99 or smaller than −0.99.

Any herbal composition can then be characterized through the use of gene expression profiles generated through the use of nucleic acid microarrays. Moreover, one can choose any number of genes that are differentially expressed to be included in the data set represented the gene expression profiles. For example one may choose about 10 genes, about 100 genes, about 500 genes, about1000 genes, about 1500 genes, abut 2000 genes, about 2500 genes or more, or any number in between.

The prescription of the Chinese herbal medicine Scute and Licorice combination (Huang Chin Tang) stops diarrhea, relieves spasms and clears fever. The ingredients of Huang Chin Tang are Scute, Peony, Licorice and Jujube. This recipe has been used for more than 1000 years but the chemical and biomedical studies on the prescription have not been carried out until recent decades. In this study we used the nucleic acid microarray technology to study the gene expression profiles of herbal medicines treated cells. Our aims are to demonstrate the feasibility of using the microarray/CD system for classification of different herbal compositions or different preparations and to find the predictor genes (marker genes) for the Huang Chin Tang prescription. The long-term goals are to find the correlation of the biochemical ingredients in each herbal composition with the gene expression profiles of various treated cells and to decipher the molecular pharmacological mechanisms of the Chinese herbal medicines in a rational fashion.

Materials and Methods.

1. Development of a Cell Banking System.

Purpose:

Microarray system is a sensitive detection method to monitor gene expression patterns of cells. It is necessary to build a Cell Banking System with a Master Cell Bank (MCB) and a Working Cell Bank (WCB) to minimize cell variability for herbal medicine testing.

Scope:

The Cell Bank System is used for all types of cells in microarray studies.

Apparatus:

CO₂ Air-Jacketed Incubator (NUAIRE™ DH autoflow)

Centrifuge (KUBOTA 2100)

Freezing vial (Corning Costar, Cat. #430659)

Tissue culture flask 750 ml (Falcon, Cat. #3045)

Tissue culture dish 150×25 mm (Falcon, Cat. #3025)

Cell:

Jurkat T cell from Dr. Alexandra Ho

Reagents:

RPMI Medium 1640 (GIBCO BRL, Cat. #31800-014)

Dimethyl Sulphoxide (DMSO) (Sigma, Cat. #D-2650)

Fetal Bovine Serum (HyClone, Cat. #SH30070.03, Lot #AGL7258)

2-mercaptoethanol (GIBCO BRL, Cat. #21985-023, 5×10⁻² M)

Media:

I. Culture medium: 90% RPM+10% fetal bovine serum+2-mercaptoethanol (5×10⁻⁵ M)

II. Freezing medium: 90% RPM 1640+10% DMSO

Procedure:

A. Master Cell Bank

1. Follow the standard sterile procedure of cell culture.

2. Seed Jurkat T cells in culture medium in flask at 37° C. with 5% CO₂ incubator.

3. After incubation for two days, count the cell number and spread the cells to two flasks. Note. Cell density is kept about 5×10⁴-2×10⁶ per ml.

4. Culture and count the cell number until the number reaches 2×10⁷.

5. Collect the cells in 50 ml centrifuge tubes and spin at 1300 rpm (300×g) for 5 min.

6. Discard the supernatant and re-suspend the cell pellet with chilled freezing media. The cell number in each vial is about 1×10⁶ per ml.

7. Slowly freeze the cells by the following temperature profile: −20° C. for 2 hr, −80° C. for 24 hr and then place the cells in liquid N₂ storage. Store a total of 20 frozen vials in MCB.

B. Working Cell Bank

1. Retrieve one vial of cell from MCB in liquid N₂ tank and quickly thaw at 37° C. water bath.

2. Transfer the cells into 10 ml of warm culture medium.

3. Spin down the cells at 1300 rpm (300×g) for 5 min. Discard the supernatant. Culture the cells with 20 ml medium in a flask.

4. Sub-culture the cells to 2 flasks.

5. Seed 5×10⁷ cells with 500 ml culture medium in each flask with stirring for a total of 2 flasks. Culture the cells for 2 days.

6. Culture until the cell density reaches 1×10⁶/ml and a total volume of 1 L.

7. Prepare freezing media by adding 100 ml of fetal bovine serum and 10 ml of DMSO.

8. Centrifuge, discard the supernatant and re-suspend the cells to 110 ml of freezing medium.

9. Dispense 1 mil to every freezing vial (10 million cells per vial) for a total of 100 vials. Slowly freeze the cells by the temperature profile described above.

2. Determination of Growth Inhibition Concentration of Herbal Extract in Cell Cultures.

Purpose:

Most drugs are toxic to cells. This experiment is designed to examine the toxicity of herbal extracts in Jurkat T cells and to determine the growth inhibition concentration of herbal extracts that keeps the cells alive.

Scope:

This assay can be used in all kinds of herbal extracts to examine the toxicity.

Apparatus:

CO₂ Air-Jacketed Incubator (NUAIRE™ DH autoflow)

Counting chamber (Hemacytometer, Reichert, USA)

Microscope (Zeiss, Axiovert 100)

Cell:

Jurkat T cell

Reagents:

RPMI Medium 1640 (GIBCO BRL, Cat. #31800-014)

Fetal Bovine-Serum (HyClone, Cat. #SH30070.03, Lot #AGL7258)

2-mercaptoethanol (GIBCO BRL, Cat. #21985-023, 5×10⁻² M)

Culture media: 90% RPMI+10% fetal bovine serum+2-mercaptoethanol (5×10⁻⁵ M)

Disposable sterile syringe filters (0.2 m, Corning, Cat. #21052-25)

Herbal extracts:

1. Cordyceps Sinensis Mycelium

2. ST 024:

3. ST 044:

4. ST 051:

5. ST 093:

6. ST 117:

7. ST 123:

8. ST 128:

9. ST 134:

10. ST 237:

11. PHY906-303503: Complex-mix composed of 4, 6, 7, 10

12. PHY906-284003: Complex mix composed of 4, 6, 7, 10

Procedure:

A. Herbal Extract Preparation

1. Dissolve 1 gram of herbal powder in 10 ml of 80° C. deionized water (neutral pH) in a polypropylene tube.

2. Incubate the tube at80° C. water bath for 30 minutes with gentle shaking then centrifuge at 4000 rpm (1500×g) for 5 min to obtain the supernatant.

3. Centrifuge at 11000 rpm (14000×g) for 10 min to collect the supernatant.

4. Using the disposable sterile syringe filter to filter the supernatant.

B. Cell Survival Test

1. Culture Jurkat T cells as described above.

2. Dispense 1ml of 5×10⁵/ml cells per well to 24-well culture plates.

3. Prepare 12 kinds of herbal extract solutions. The extract solutions must be freshly prepared and used immediately.

4. Add 100, 50, 20, 10, 5 μl of each herbal extract solution into the 24 well culture plates to get the five different concentrations: 10, 5, 2, 1, 0.5 mg/ml.

5. Culture the cells for24 h at 37° C. in an incubator filled with 5% CO₂.

6. Count the number of cells per well. Mix 10 μl of cell solution with 10 μl of Trypan blue dye and load into cell counting chamber.

7. Count the four major square areas to calculate the cell number. (number of cells in 4 areas)/4×10⁴×dilution factor=number of cells per ml

3. Profiling Gene Expression Patterns of Jurkat T Cells Treated With Herbal Extracts.

Purpose:

Profile the gene expression patterns of Jurkat T cells treated with herbal extracts. A high-density nucleic acid microarray with colorimetric detection system is used.

Apparatus:

Heat block (Boekel, Model 110002)

Spectrophotometer (Beckman, DV640)

Centrifuge (KUBOTA 1910)

Water bath (SLM AMINCO, Model 800)

Hybridization incubator (YIH DER OH-800)

Heat sealer (TISH-300, TEW)

Reagents: RNAzol™ B (Tel-Test, Cat. #CS-104)

Oligotex mRNA Midi Kit (Qiagen, Hilden, Germany)

Hybridization Bags (GIBCO BRL, Cat. #18278-010)

EasiSeal (Hybaid, Cat: #HBOSSSEZ1E)

Glass slides (Matsunami, S2214, Japan)

Aerosol Resistant Tips (ART tip) (molecular BIO-Products, Cat. #2139)

Random hexamer primer (GIBCO BRL, Cat. #48190-011)

Reverse transcriptase and 5× buffer (GIBCO BRL, Cat. #18064-014)

RNase inhibitor (GIBCO BRL, Cat. #10777-019)

Biotin-16-dUTP (Boehringer Mannheim, Cat. #1093070)

Dig-11-dUTP (Boehringer Mannheim, Cat. #1558706,)

Blocking powder for hybridization (Boehringer Mannheim, Cat. #1096176)

Bovine serum albumin (Sigma, Cat. #A2153)

20×SSC (Amresco, Cat.#0918S-2-20XPTM5L)

SDS (Merck, Cat. #113760)

Dextran Sulfate (Sigma, Cat. #D6001)

Streptavidin-β-galactosidase (GIBCO BRL, Cat. #19536-010)

Anti-dioxigenin-AP Fab fragments (Boehringer Mannheim, Cat. #1093274,)

X-gal (GIBCO BRL, Cat. #15520-018)

Maleic acid (Sigma, Cat. #M1125)

N-lauroylsarcosin (Sigma, Cat. #L5777)

Fast red TR/AS-MX substrate kit (PIERCE, Cat. #34034)

Polyethylene glycol (Sigma, Cat.#P2139)

Reagent Preparation:

1 × hybridization buffer (4X SSC,
0.1% N-lauroylsarcosine, 0.02% SDS, 1% BM
blocking reagent)

| | |
|---|---|
| 20 × SSC | 16 m |
| 1% N-lauroylsarcosine | 8 ml |
| 10% SDS | 160 μl |
| BM blocking powder | 0.8 g |
| H₂O | 51 ml |
| total | 80 ml |

Heat to 65° C. to dissolve the powder then store at −20° C.

50% PEG-8000 (Polyethylene Glycol)

| | |
|---|---|
| PEG-8000 | 10 g |
| H₂O | up to 20 ml |

Heat to 65° C. to dissolve then autoclave. Aliquot and store at −20° C.

| | |
|---|---|
| 10 × TBS (100 mM Tris, | |
| 1.5 M NaCl, pH 7.4) | |
| Tris base | 12.1 g |
| NaCl | 87.6 g |
| H₂O | up to 1000 ml |
| 120 mM X-gal | |
| X-gal | 100 mg |
| DMF | 2 ml |

Store at −20° C.

| | |
|---|---|
| X-gal Substrate Buffer (1 mM MgCl₂, | 500 ml |
| 3 mM K₃Fe(CN)₆, | |
| 3 mM K₄Fe(CN)₆ in 1X TBS | |
| buffer) | |
| 10X TBS/pH7.4 | 50 ml |
| Potassium Ferrocyanide | 633.5 mg |
| Potassium Ferricyanide | 493.9 mg |
| MgCl₂ | 101.6 mg |

Filter and store at −20° C.

BM Blocking Dilution Buffer/pH7.5
(0.1 M maleic acid, 0.15 M NaCl)

| | |
|---|---|
| 1M Maleic Acid | 100 ml |
| 5M NaCl | 30 ml |
| Solid NaOH | 7.5 g |
| H₂O | up to 1000 ml |
| 10% Blocking Reagent | 100 ml |
| Blocking Powder | 10 g |
| Blocking Dilution Buffer | 100 ml |
| (no tween 20) | |

Heat to 70° C. then autoclave. Store at 4° C.

20% Dextran Sulfate

| | |
|---|---|
| Dextran Sulfate | 2 g |
| H₂O | up to 8 ml |

Autoclave then store at −20° C.

| | |
|---|---|
| DEPC-treated water | 800 ml |
| Diethyl Pyrocarbonate (store at 4° C.) | 400 μl |
| H₂O | 800 ml |

Put in 37° C. shaking water bath for 4 hrs and then in 37° C. warm room for overnight. Autoclave the solution for 45 minutes or 25 minutes each for two times.

Procedure:

A. Preparation of Jurkat T Cells

1. Thaw one vial of Jurkat T cells. Transfer to 10 ml growth medium. The number of vials to thaw depends on the number of test to perform. In general, one vial of cells is needed for 2 herbal extract tests.

2. Re-suspend the cells in 50 ml of culture medium.

3. Incubate the cells for one day. Add 150 ml of culture medium and divide into two flasks with 100 ml each.

4. Culture the cells for 3 days.

5. Change the medium and distribute 4 flasks with 100 ml of medium each.

6. Culture the cells for 2 days.

7. Count the cell number. Collect the cells and spin down. Re-suspend the cell pellet with culture medium to 5×10⁵ cells per ml and 100 ml per flask.

8. Culture the cells for 3 hrs before adding the herbal extract.

B. Herbal extract treatment

1. Prepare the herbal extract (see herbal extract preparation).

2. According to the growth inhibition concentration of herbal extract determined by the cell survival experiments, calculate the 50% growth inhibition concentration of each herbal extract.

3. Define the 50% growth inhibition concentration as H. Treat the cell with serial dilution of herbal extracts with the following concentrations: H, H/2.5, H/5, H/10, H/20.

4. Culture the cell for 24hrs.

5. Collect cells and count, the number of cells. Centrifuge to obtain cell pellet.

6. Wash the cell pellet with 1× PBS once.

7. Discard the supernatant. The cell pellet is ready for total RNA isolation.

C. Isolation of total RNA

1. Add 1 ml of RNAzol™ B per 10⁷ cells. Homogenize the cell pellet but do not vortex.

2. Add 0.1 ml chloroform per ml of homogenate, cover the samples tightly, shake vigorously for 1 min (do not vortex). Place on ice for 15 min.

3. Centrifuge at 12000 rpm (13500×g) at 4° C. for 15 min.

4. After centrifugation, the homogenate develops two phases: a lower blue phenol-chloroform phase and a colorless upper aqueous phase. DNA and proteins are in the interphase and the organic phase. Transfer the aqueous phase to a new tube, add an equal volume of isopropanol and store the samples at −80° C. Note. The range of isopropanol addition is from 0.7 to 1 volume of the aqueous phase solution.

5. Keep the samples at −80° C. until use. Let the sample completely thaw before centrifuging and mix 2 to 3 times by inverting the tube. Centrifuge samples for 15 min at 13000 rpm (15000×g).

6. Remove the supernatant and wash the RNA pellet once with 1 ml of 75% ethanol. Centrifuge for 3 min at 13000 rpm (15000×g) and at 4° C.

7. Discard the supernatant. Dry the pellet under vacuum for 1 min. Note. Do not let the RNA pellet dry completely. It will greatly decrease its solubility.

8. Dissolve the RNA-pellet in 50-100 μl of diethylpyrocarbonate (DEPC)—treated water by pipetting. Note. If the pellet is hard to dissolve, incubating the pellet for 10-15 min at 60° C. may help.

9. Measure absorbance at 260 nm (A₂₆₀) and 280 nm (A₂₈₀) with a spectrophotometer. Concentration analysis OD₂₆₀×40 ng/μl×dilution factor=total RNA (ng/μl).

D. Isolation of poly-A+ mRNA from total RNA

1. Determine the amount of starting RNA and the appropriate volume of Buffer OBB and Oligotex suspension solution to be added in the RNA solution according to the Table 17.

TABLE 17
Buffer amounts for Oligotex mRNA Spin-Column Protocol.

| Total | | Add RNase-free | Buffer OBB | Oligotex | Prep |
|---|---|---|---|---|---|
| RNA | | water to: (μl) | (μl) | Suspension (μl) | size |
| 20 | μg | 100 | 100 | 6 | Mini |
| 0.25 | mg | 250 | 250 | 15 | Mini |
| 0.25-0.5 | mg | 500 | 500 | 30 | Midi |
| 0.5-0.75 | mg | 500 | 500 | 45 | Midi |
| 0.75-1 | mg | 500 | 500 | 55 | Midi |

The following procedures are based on using 500 μg total RNA as an example.

2. Add 500 μl of 2× Binding Buffer and 30 μl Oligotex Suspension to the total RNA sample. Mix the contents thoroughly by flipping the tube.

3. Incubate the sample for 10 min at 70° C.

4. Incubate for 20 min at room temperature.

5. Centrifuge for 2 min at maximum speed (14000 to 18000×g) and aspirate the supernatant.

6. Re-suspend the pellet in 400 μl of Wash Buffer OW2 and transfer onto spin column and centrifuge the spin column for 1 min.

7. Wash with 400 μl of OW2 and centrifuge as above.

8. Add 20 μl of preheated (70° C.) Elution Buffer onto the column and re-suspend the resin. Close the microcentrifuge tube.

9. Put the spin column with 1.5 ml microcentrifuge tube at 70° C. for 3 min.

10. Centrifuge the column at maximum speed for 2 min at room temperature.

11. Elute again. (repeat step 8 to 10 to get better yield).

E. cDNA Labeling

1. Mix 2 μg mRNA, 1 μl of control plants mRNA for single color label (Hat22: 1×10⁹, Rbcl: 5×10⁸, Ga4: 1×10⁸, Rca: 5×10⁷, Asa1: 1×10⁷, Atps: 5×10⁶ molecule/μ), 6 μl of 50 mM random hexamer and DEPC-H₂O to 28.88 μl final volume. For dual-color mode, use 2 μg of mRNA each in Biotin or Dig labeling and individual addition of control plants mRNA: 1 biotin labeling: Hat22: 1×10⁸, Rbcl: 5×10⁷, Ga4: 2×10⁷, Rca: 1×10⁷, Asa1: 1×10⁷, Atps: 1×10⁷, Hat4: 1×10⁷/μl. 2. Dig labeling: Hat22: 1×10⁷, Rbcl: 1×10⁷, Ga4: 1×10⁷, Rca: 1×10⁷, Asa1: 1×10⁸, Atps: 5×10⁷, Hat4: 2×10⁷/μl.

2. Denature for 10 min at 70° C., then chill quickly in ice for 5 min.

3. Add 10 μl of 5× first strand buffer, 5 μl of 0.1 M DTT, 1 μl of 25 mM dATP, dCTP, dGTP mixture, 1 μl of 2 mM dTTP, 2 μl of 1 mM biotin-16-dUTP, or Dig-11-dUTP (1 mM), 0.63 μl of 40 U/μl RNAsin and 1.5 μl of Superscript II (reverse transcriptase, GIBCO BRL)(200 U/μl).

4. Mix well and incubate for 10 min at 25° C., then for 90 min at 42° C.

5. Stop the reaction for 5 min at 94° C.

6. Add 5.5 μl of 3 M NaOH for 30 min at 50° C.

7. Add 5.5 μl of 3 M CH₃COOH for 30 min at 50° C.

8. Precipitate the labeled cDNA by adding 34 μl of water, 50 μl of 7.5 M ammonia acetate, 10 μg of linear polyacrylamide as carrier and 380 μl of absolute alcohol.

9. Incubate the sample for 30 min at ×80° C. Centrifuge at 13000 rpm for 15 min.

10. Wash the pellet with 1 ml of 70% ethanol and centrifuge at 13000 rpm for 5 min.

11. Dissolve the pellet in 36 μl of autoclaved H₂O. For dual color, combine two labeled cDNA together.

F. Array Hybridization

1. The filter membrane carrying the 9600 EST PCR products is pre-hybridized in 5 ml of 1× hybridization buffer (4×SSC, 0.1% N-lauroylsarcosine, 0.02% SDS, 1% BM blocking reagent (Boehringer Mannheim)), and 50 μg/ml salmon sperm DNA (GIBCO BRL) at 63° C. for 1.5 hours. Note. You can prepare 80 ml of 1× hybridization buffer and store it at −20° C. Thaw the buffer at 60° C. before use.

2. Stick one side of adhesive EasiSeal® square to a clean glass slide and place the pre-hybridized membrane in the center of the square with the spots facing up.

3. Mix the probe with 2 μl of poly-d(A)10 (10 μg/μl) and 2 μl of human Cot-1 DNA (10 μg/μl) (GIBCO BRL) and 40 μl of 2× hybridization buffer to 80 μl final volume.

4. Denature the probe mixture at 95° C. for 5 min and then cool on ice.

5. Seal the filter membrane with the probe solution in the hybridization bag.

6. Incubate at 95° C. for 5 min and then at 63° C for 12-16 h (overnight).

7. Wash the filter membrane twice with 5 ml of 2×SSC, 0.1% SDS for 5 min at room temperature.

8. Wash three times for 15 min each with 5 ml of 0.1×SSC, 0.1% SDS at 63° C.

9. Block the filter membrane with 5 ml of 1% BM blocking reagent containing 2% dextran sulfate at room temperature for 1 h.

10. Incubate with 5 ml mixture containing 700× diluted Streptavidin-β-galactosidase (1.38 U/ml, enzyme activity)(GIBCO BRL), 10000× diluted anti-Digoxigenin-alkaline phosphatase (0.075 U/ml, enzyme activity)(Boehringer Mannheim), 4% polyethylene glycol 8000 (Sigma), and 0.3% BSA in 1× TBS buffer at room temperature for 2 hours. Note. This formula is for dual-color mode. For single color mode, anti-Dig-AP is not needed and the incubation time can be reduced to 1 hour.

11. Wash with 1× TBS buffer three times for 5 minutes each.

12. Freshly prepare X-gal substrate solution (1.2 mM X-gal, 1 mM MgCl₂, 3 mM K₃Fe(CN)₆, 3 mM K₄Fe(CN)₆ in 1× TBS buffer) by mixing 50 μl of 120 mM X-Gal and 5 ml of X-Gal substrate buffer. Immerse the filter membrane in the X-gal substrate solution for 45 min at 37° C. with gentle shaking.

13. Wash with 1× TBS.

14. Dual color development: stain the membrane with 5 ml of Fast red TR/naphthol AS-MX substrate (Pierce, Rockford, Ill.) at room temperature for 30 minutes with gentle shaking.

15. Wash with deionized water. Stop the reaction with 1× PBS containing 20 mM EDTA for 20 min.

16. Air dry the filter membrane.

Results.

1. Determine the Growth Inhibition Concentrations of Herbal Extracts in Cell Cultures.

Each herbal extract has different cellular toxicity, thus it is necessary to determine the growth inhibition concentration of every herbal medicine before treating the cells. Five serial dilutions of herbal extract (10, 5, 2, 1, 0.5 mg/ml) were added to 5×10⁵/ml cultured cells and incubated for 24 hours in an incubator at 37° C. with 5% CO₂. The numbers of survival cells at different concentrations of herbal extracts are shown in Table. 18.

TABLE 18
Survival Cell Number at Different
Concentrations of Herbal Extracts

| | | | | | | | | experimental | |
|---|---|---|---|---|---|---|---|---|---|
| | | | | | | | | concentration | |
| | | | | | | | No | High | |
| | | | | | | | | conc. | Low conc. |
| | no. × 10⁵/ml | 10 mg/ml | 5 mg/ml | 2 mg/ml | 1 mg/ml | 0.5 mg/ml | drug | (mg/ml) | (mg/ml) |
| 1 | Cordyceps | 8.4 | 11.9 | 11.5 | 9.2 | 9.0 | 12.4 | 10 | 1 |
| | sinensis | | | | | | | | |
| | mycelium | | | | | | | | |
| 2 | ST024 | 4.2 | 8.7 | 10 | 7.5 | 10 | 10 | 10 | 1 |
| 3 | ST044 | — | 5.9 | 8.4 | 9.9 | 9.4 | | 5 | 0.5 |
| 4 | ST051 | — | — | — | 1.7 | 5.4 | | 0.5 | 0.05 |
| 5 | ST093 | — | — | 1.9 | 3.8 | 4.4 | | 0.5 | 0.05 |
| 6 | ST117 | — | 1.6 | 3.6 | 4.6 | 5.8 | | 0.5 | 0.05 |
| 7 | ST123 | 3.4 | 6.4 | 8 | 9.3 | 7.8 | | 5 | 0.5 |
| 8 | ST128 | 3.5 | 7.7 | 7.9 | 7.7 | 8.3 | | 5 | 0.5 |
| 9 | ST134 | 2.9 | 6.1 | 11.2 | 9.6 | 9.8 | | 5 | 0.5 |
| 10 | ST237 | — | — | 2.5 | 6.6 | 8.7 | | 1 | 0.1 |
| 11 | PHY906-303503 | — | — | 4.5 | 4.9 | 8.2 | | 1 | 0.1 |
| 12 | PHY906-284003 | — | — | 3.8 | 5.7 | 7.6 | | 1 | 0.1 |

Note:
The original cell number was 5 × 10⁵/ml. The number increased to 10 × 10⁵/ml after 24 h incubation.
“—” indicates all dead cells.

The cell number without herbal extract addition doubled after 24 hours incubation. On the other hand, the number of survival cells varies with different herbal medicine treatments. We chose the 50% growth inhibition concentration (IC₅₀) as the high concentration and one-tenth of it as the low concentration. In order to maintain consistence of the Jurkat T cell line, a cell banking system was established. In the cell bank, a total of 100 vials of cells (10 million cells per vial) were frozen in a −150° C. freezer.

2. Molecular Classification of Herbal Medicines by Nucleic Acid Microarray Analysis.

Analysis of 3 single-element herbal medicines. Three single-element herbal medicines, Cordyceps Sinensis Mycelium (CSM), ST024, and ST117 were used to treat the cell cultures as described in the methods section. Gene expression measurements were performed by using microarrays of 13824 cDNA fragments each representing a distinct human transcript. For the data analyses, gene spots of high data quality were selected. The selection was based on signal to background ratio greater than 2.5 or the coefficient of variation (CV) of spot area smaller than 10%. All the data sets were normalized with the control cells, which received no herbal treatments. The spot intensity was rounded up to 10 for those intensities that were less than 10. Based on the selection criteria, a total of 492 genes with differential expression ratio greater than 1.5 were selected for cluster analysis. These data pre-processing procedures were performed by the program. “DataExtract” and “Ratio2” developed in-house.

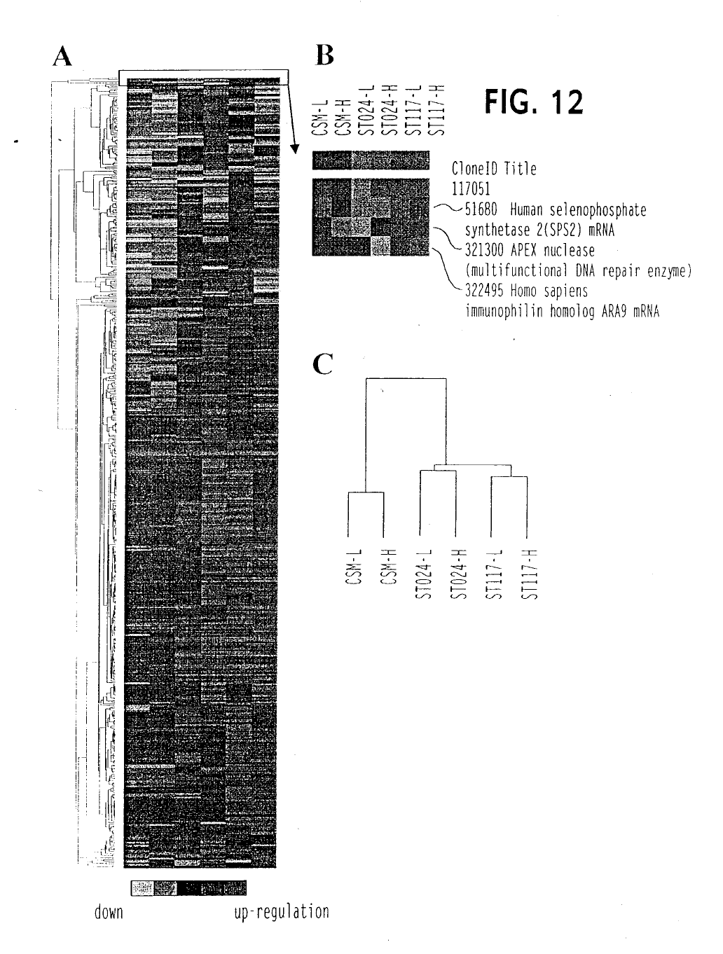

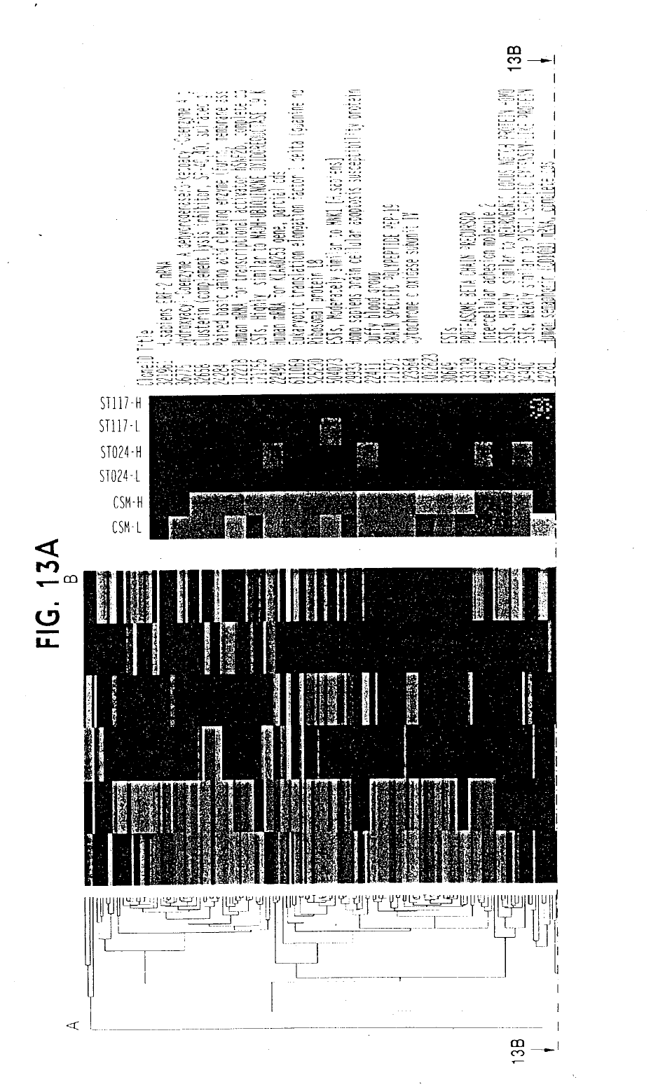

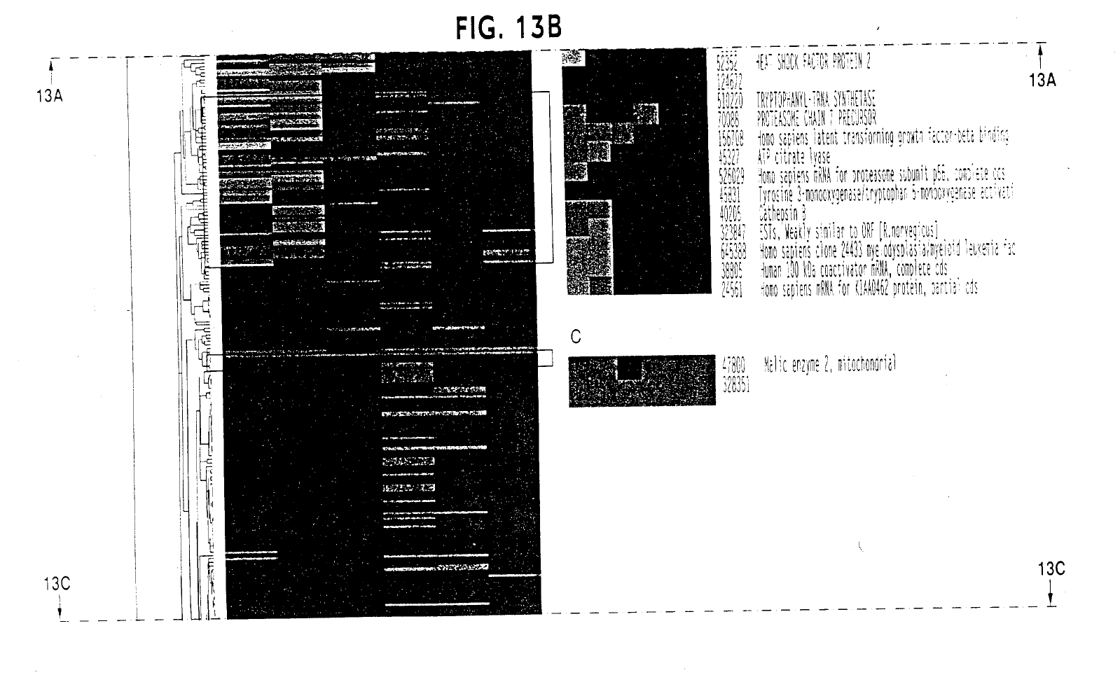

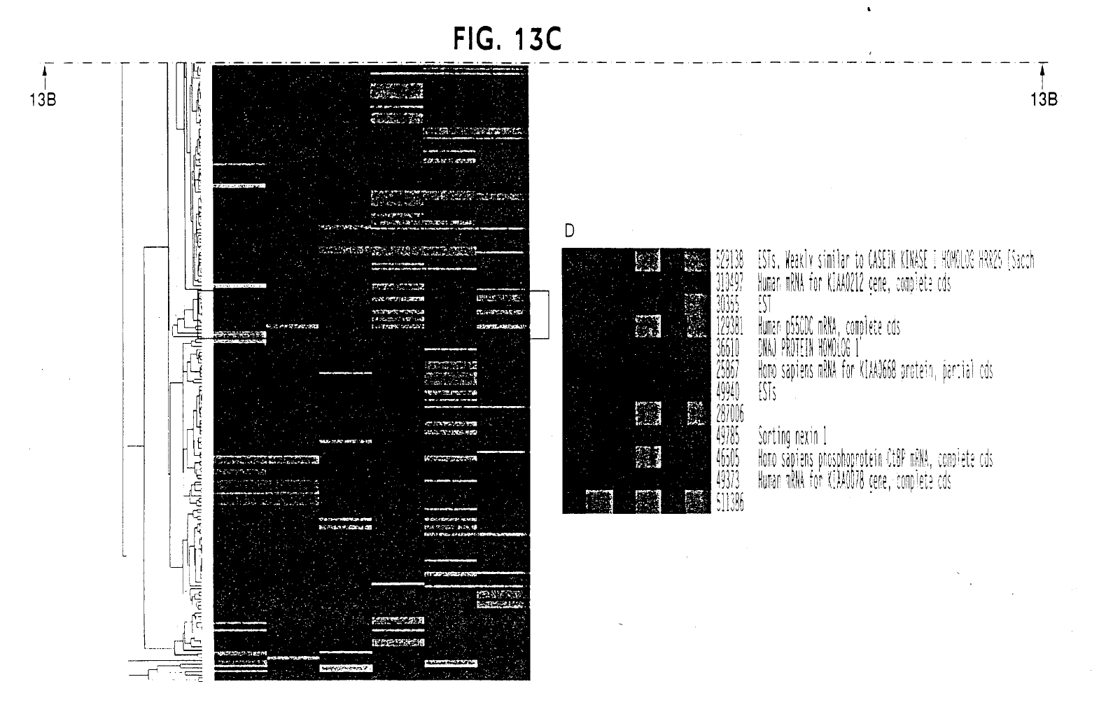

These 492 genes were cluster analyzed by the average-linkage method. The distance between genes is used as the linear correlation coefficient or resemblance coefficient. The cluster analysis programs, Cluster and TreeView, were based on hierarchical clustering method and were written by Dr. Michael Eisen of Stanford University (Eisen, 1999, Eisen et al., 1998). The results are shown in FIG. 12. From FIG. 12C, one can clearly identify that three different herbal medicines of high and low concentrations are each clustered together. For instance, CSM-L is closer to CSM-H and less similar to ST024 or ST117 in the clustering tree. A different clustering algorithm, the self-organizing map algorithm, which is based on non-hierarchical method, yields the same results (data not shown). From the clustering results shown in FIGS. 12A & 13, several features are noted. (1) 4 genes were up regulated by ST117 treatment but down regulated by other herbal treatments (FIG. 12B). (2) 34 genes were down regulated by CSM treatment but up regulated by others (FIG. 13B). (3) 2 genes were up regulated by all the three herbal treatments, one is Malic enzyme 2 and the other one is an anonymous gene (clone ID 328351) (FIG. 13C). (4) 12 genes were highly induced by the high concentration treatment and less induced by the low concentration treatment in all the three herbal medicines (FIG. 13D).

Analysis of 2 preparations of multi-element herbal medicines. Two batches of Huang Chin Tang, PHY906-303503 (#11) & PHY906-284003 (#12), each with low and high concentrations were used to treat the cell cultures in three independent experiments. The gene expression profiles were acquired with microarrays of 9600 non-redundant cDNA elements. After the data pre-processing procedures as described above about 5000 genes were selected for the subsequent data analysis. There were 3 repeats for each herbal treatment. For data analysis, we use a modified method based on the one reported by Slonim et al. (Slonim, 1999). The following algorithm is designed to search for the candidate marker genes that have high differential expression ratios but low deviation in the three repeats. We designate a P(i) value to account for the gene i with the aforementioned features.

P(i)=square root of(Σ(μₘ−μ_{c})²)/(σ_{c}+Σσₘ)

μ: Mean expression levels in three repeat experiments for herbal treated cells (μₘ) or untreated control cells (μ_{c}).

σ: Standard deviation of the expression levels in three repeat experiments for herbal treated cells (σₘ) or untreated control cells (σ_{c}).

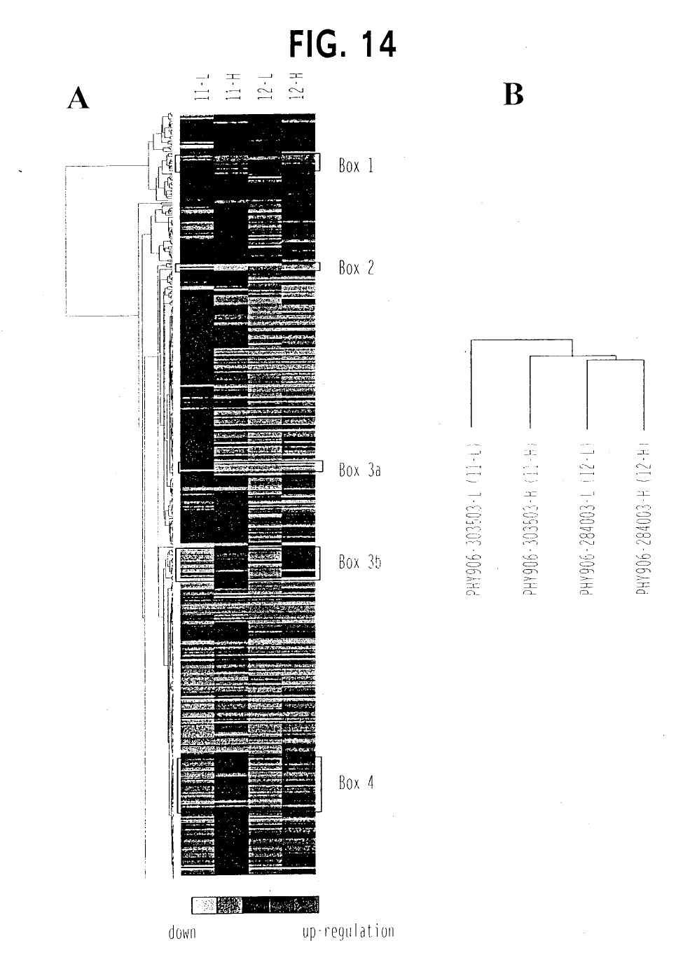

We calculated the P(i) value for each gene and selected 500 genes with the highest scores as candidate genes for cluster analysis (FIG. 14). The values of each gene were averaged over the 3 repeats. As shown in FIG. 14B, two different concentrations of #12 are clustered together (12-H & 12-L). The higher concentration of the #11 preparation is closer to the #12 preparation cluster than the lower concentration of #11 preparation. However, all these clusters have similar resemblance coefficient (distance between clusters) compared with the tree shown in FIG. 12. These results suggest that the gene expression profiles of #11 and #12 preparations of Huang Chin Tang are similar. The results are justified based on the fact that these two preparations are based on the same herbal medicine mix.

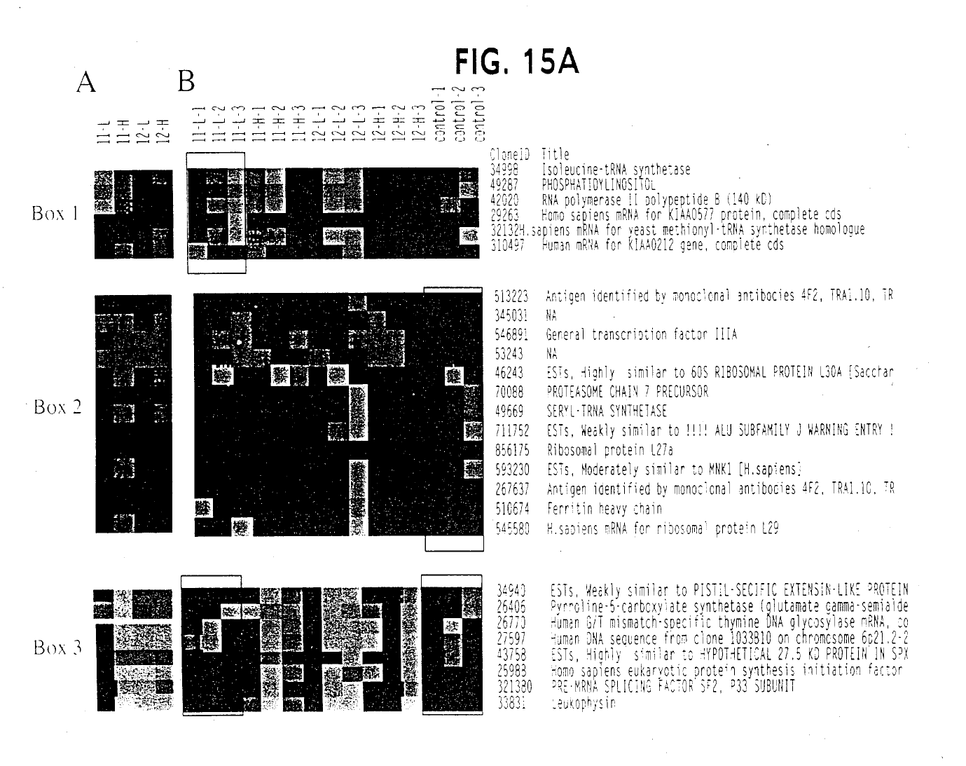

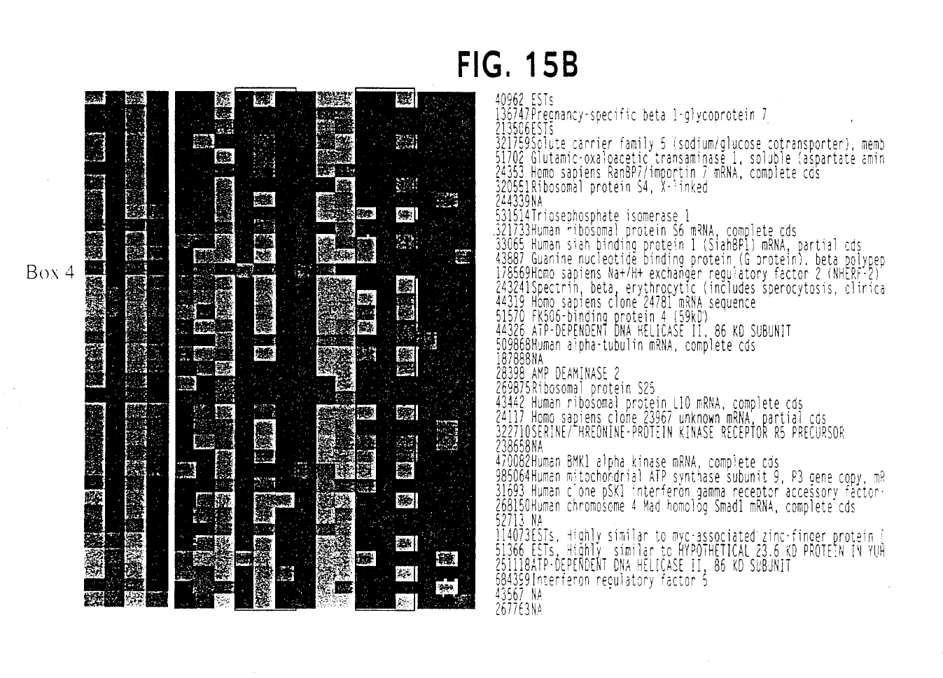

Several features are noted in the expression profiles illustrated in FIGS. 14A & 15. The averaged gene expression levels are shown in FIG. 15A The Box1 encloses genes that were down regulated in #11-L treated cells but up-regulated in others. These genes include 2 tRNA synthetase (isoleucine and methion), RNA polymerase II polypeptide B (Clone ID 42020), KIAA0212 gene (Clone -ID 310497, containing ATP/GTP-binding site motif A), and KIAA0577 (Clone ID 29263, ATP-dependent RNA helicase). It is interesting to note that 3 out of the 6 genes were involved in the RNA replication. Box2 encloses the genes that were up regulated by all the #11 and #12 treatments. Box3 encloses the genes which showed no response by #11-L treatment but were down regulated by the others. Box4 encloses the genes that were highly repressed by low concentration herbal treatment but were less repressed by high concentration herbal treatment. Finally, in Box1 and Box3, the expression profiles of #11 treated cells are different from the profiles generated by the other 3 treatments. This result is consistent with the finding depicted in FIG. 14B.

Combining the data sets of the gene expression profiles of the 3 single-elements and the 2 preparations of multi-element herbal medicines together, a couple features are noted as an illustration. The KIAA022 gene (Clone ID 310497, containing ATP/GTP-binding site motif A) was highly induced by all the high concentration herbal treatments except that it was only mildly induced by the #11-L and the CSM treatments. Two genes, an anonymous gene, (Clone ID 510908) and Proteasome chain 7 precursor (Clone ID 70088) were highly up-regulated on all the treatments except down-regulated by the CSM treatment.

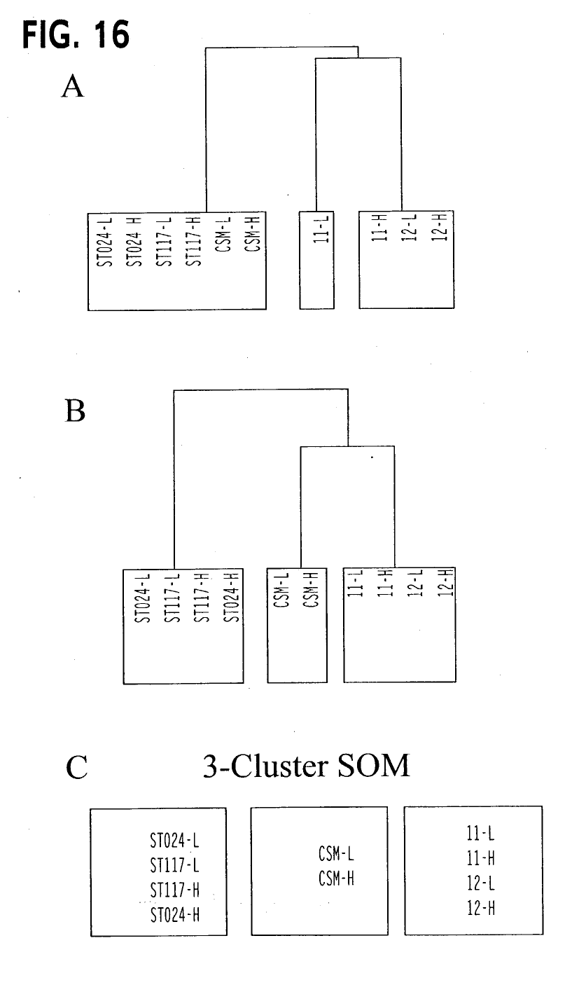

We next worked on the crux to cluster analyze the gene expression profiles of the 5 different types of herbal medicine treatments. The data pre-processing procedures were performed as aforementioned and 500genes were selected for cluster analysis. A hierarchical clustering was performed by the program “Cluster” described above. The hierarchical tree was cut at the position where the range of distance between clusters is the greatest (Romesburg, 1989) and the result is shown in FIG. 16A. The 3 single-element herbal medicines, CSM, ST024, and ST117 are clustered together. Out of the 2 different batches of the multi-element herbal medicine, #12-H, #12-L and #11H are clustered together and the #11-L stands by itself. The result suggests that higher similarity exists between the #11 and #12 as compared with that of CSM, ST024, and ST117. In order to better classify the different herbal medicines, the data analysis algorithm was improved by standardizing all the data sets so that the expression level of each gene across the different data sets has zero-mean and unit-variance (Tavazoie et al., 1999; Chen et al., 1999). This yields the transformed variables:

χᵢ=(x_{I}−μₓ)/σₓ

μₓ: mean expression levels in the data set

σₓ: standard deviation of the expression levels in the data set

xᵢ: un-transformed gene expression level

χᵢ: transformed gene expression level

After standardizing the data sets #11 and #12 are clustered together as shown in FIG. 16B. The ST024 and ST117 are clustered together and the CSM is in an independent cluster. Furthermore, the clustering also suggests that CSM is more similar to #11 and #12 than to the ST024 and ST117. Another clustering algorithm, self-organizing maps, was performed with the same standardized data sets and yields the same result as the hierarchical clustering (FIG. 16C).

Class predictors for discriminating #11 and #12 herbal treated expression profiles. The above cluster analyses for the #11 and #12 show that they are similar and further classification is difficult by the hierarchical clustering or self-organizing maps methods with the data set containing the 500 genes of the highest P(i) values. We then modified the algorithm to select genes with larger expression ratio difference between #11 and #12 herbal treated cells, but smaller variation in the two herbal treated cells. The T(i) value is defined to score this feature as following:

T(i)=log(μ₁₁)−log(μ₁₂)/(σ₁₁+σ₁₂)

μ: Mean expression ratios in three time experiments for #11 treated cells (μ₁₁) or #12 treated cells (μ₁₂)

σ: Standard deviation of the expression ratios for #11 treated cells (σ₁₁) or #12 treated cells (σ₁₁)

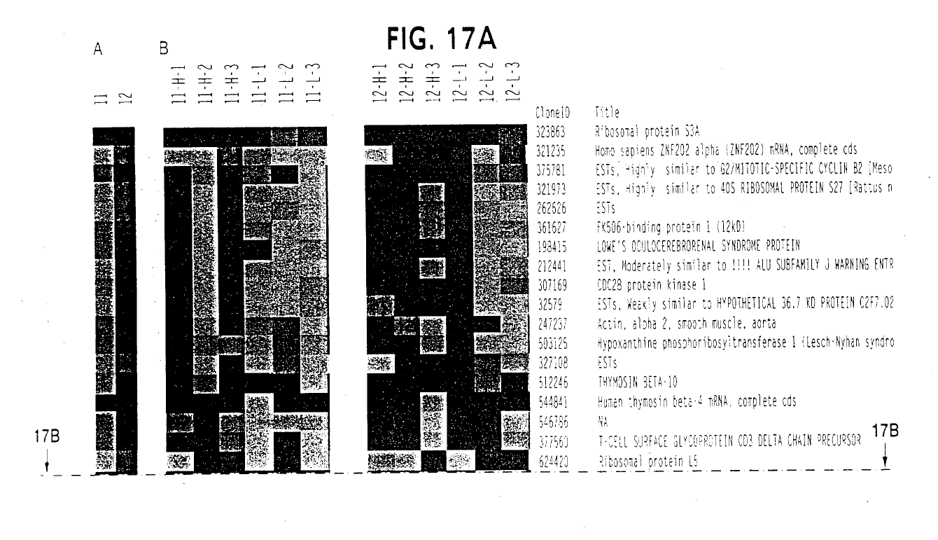

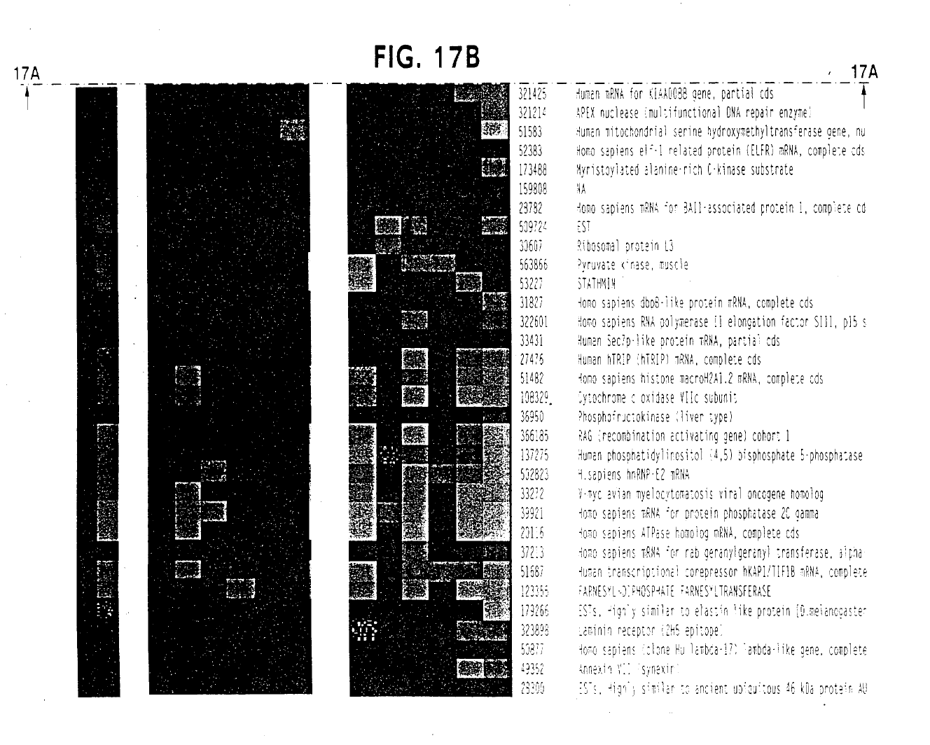

We calculated the T(i) value for each gene and selected 50 genes with the highest scores as class predictors (FIG. 17). 18 genes were up-regulated by the #11 treatment and down-regulated by the #12 treatment. The rest of the genes were up-regulated by the #12 treatment and down-regulated by the #11 treatment. We then used these class predictors to classify two test herbal preparations based on a modified method described by Golub et al., 1999.

Two different batches of Huang Chin Tang preparations, PHY010401 (#16) & PHY010402 (#17) were obtained from Sun Ten Pharmaceutical Co. and were used for the class prediction test. The gene expression profiles of #16 and #17 preparations were normalized with the expression profile of the untreated control cells and standardized with the class predictors. Each predictor gᵢ votes for either #11 or #12 herbal preparation depending on whether its expression level xᵢ is closer to #11 or #12. The vote for each gene is given by vᵢ=|x_{I}−(μₘ+μ_{c})/2|, where

μ: Mean expression ratio in three repeat experiments for #11 (μ₁₁) or #12 herbal treated cells (μ₁₂)

The average votes V₁₁ and V₁₂ were collected from the predictor genes correlated with the predictor on #11 and #12, respectively. The prediction strength (PS) reflects the margin of victory and it was defined as PS=(V₁₁−V₁₂)/(V₁₁+V₁₂). If the PS was greater than 0, it indicated that the herbal preparation was more similar to #11 and less similar to #12. The results obtained from the analyses on #16 and #17 indicated that #16-H was similar to #11 (PS=0.1) and #16-L, #17-H, and #17-L were similar to #12 (PS=0.29, −0.21 and −0.2, respectively). Based on the information of #16 and #17 preparations, this test failed to correctly identify #16-L as more similar to #11.

Discussion.

Characteristic gene expression profiles for herbal medicines treated cells. The predictor genes are selected from the differentially expressed genes in two herbal treated cells. These genes represent the cellular responses to the herbal medicine treatments. In this study, we have identified some interesting genes based on their responses across different herbal treatments (FIGS. 13, 15, and 17). These genes are valuable assets in studying the signaling pathways of cells in response to the herbal stimulation and in deciphering the molecular pharmacological mechanism of herbal medicines.

Classification of herbal medicines by nucleic acid microarray analysis. In summary, a two-step classification procedure is proposed. An initial classification procedure based on the standardized data sets and the clustering algorithms is performed and followed by a final classification procedure with the class predictors. All the procedures can be integrated in a computer program. In these preliminary studies, all the genes have the same contribution for classification. When the data sets are large enough, the weight for each gene (or predictor) can be acquired from the linear correlation coefficient (Golub et al., 1999, Chen et al., 1999).

The #11-L failed to be clustered with #11-H by significant association. A similar preparation of #11, the #16 preparation yielded the same results. It was interesting to discover that no matter what clustering algorithms were applied, the #11 and the #16 preparations did not yield the expected results. Even with independent experiments, the results remained the same. The reason behind the failure will be investigated with detailed information of #11 and #16 preparations.

Quality control and analysis in microarray system. The quality of acquired microarray data and the choice of the statistical analysis methods are both important factors for achieving meaningful results. We have recognized that the variation among arrays contributes to errors in measuring gene expression levels. Based on the data in this report, we have found that for every herbal preparation, the high concentration treated expression profiles always cluster with its lower concentration counterpart (FIGS. 16B and 16C) and we could classify the ST117, ST024, CSM and Huang Chin Tang with two different clustering methods. In the past three months, the array quality has been improved to have less than 7% CV. We have also set up a standard procedure for assessing the quality of every batch of arrays fabricated in the lab. All these experimental findings and improvements in the microarray technologies suggest that classification and characterization of the Chinese herbal medicines by microarray system are feasible.

### References for Example 15.

Bertucci-F; Bernard-K; Loriod-B; Chang-Y C; Granjeaud-S; Bimbaum-D; Nguyen-C; Peck-K; Jordan-B R (1999) Sensitivity issues in DNA array-based expression measurements and performance of nylon microarrays for small samples Human Mol. Genetics 8(9): 1715-1722.

Bittner-L, Trent-J, Meltzer-P (1999) Data analysis and integration: of steps and arrows. Nature Genet. 22, 213-215.

Brown-P O; Botstein-D (1999) Exploring the new world of the genome with DNA microarrays. Nature genetics 21 (1) supplement, 33-37.

Chen-J J; Wu-R; Yang-P C; Huang-J Y; Sher-Y P; Han-M H; Kao-W C; Lee-P J; Chiu-T F; Chang-F; Chu-Y W; Wu-C W; Peck-K (1998) expression patterns and isolating differentially expressed genes by cDNA microarray system with colorimetry detection. Genomics. 51: 313-24.

Chen-Y, Bittner-M, Dougherty-E R (1999) Issues associated with microarray data analysis and integration. Information supplementary to article by Michael Bittner, Jeffrey Trent and Paul Meltzer (Nature Genet 22, 213-215).

Duggan-D J; Bittner-M; Chen-Y; Meltzer-P Trent-J M (1999) Expression profiling using cDNA microarrays. Nature genetics 21 (1) supplement, 10-14.

Eisen-M (1999) Cluster and Treeview manual. (rana.stanford.edu/software)

Eisen-M, Spellman-P T, Brown-P O, Botstein-D. (1998) Cluster analysis and display of gene-wide expression patterns. Proc. Natl. Acad. Sci. USA 99:14863-14868.

Golub-T R, Slonim-D K, Tamayo-P, Huard-C, Gaasenbeek-M, Mesirov-J P, Coller-H, Loh-M L, Downing-J R, Caligiuri-M A, Bloomfield-C D, and Lander-E S (1999) Molecular classification of cancer: class discovery and-class prediction by gene expression monitoring. Science October 15: 531-537.

Lander-E S (1999) Array of hope. Nature-genetics 21 (1) supplement, 3-4.

Romesburg-H C (1989) Cluster analysis for researchers. Chapter 16: How to make classifications. P203-216, Krieger Publishing Co. Malabar, Fla., USA.

Slonim-D K, Tamayo-P, Mesirov-J P, Golub-T R, Lander-E S (1999) Class prediction and discovery using gene expression data. (www.genome.wi.mit.edu/MPR).

Tavazole-S, Hughes-J D, Campbell-M J, Cho-R J, Church-G M. (1999) Systemic determination of genetic network architecture. Nature genetic 22: 281-285.

### Example 16

Identify Characteristic Gene Expression Profiles Induced by an Herbal Medicine

As stated in Example 15, the prescription of the Chinese herbal medicine Scute and Licorice combination (Huang Chin Tang) stops diarrhea, relieves spasms and clears fever. The ingredients of Huang Chin Tang are Scute, Peony, Licorice and Jujube. In this study we used the nucleic acid microarray technology to study the gene expression profiles induced by herbal medicines in mammalian cells. To investigate the characteristic expression profiles induced by the Huang Chin Tang, Jurkat T cells were treated with 5 batches of Huang Chin Tang (PHY01040; #16, PHY010402; #17, PHY03061; #18, PHY03062; #19 and PHY02231; #20 obtained from Sun Ten Pharmaceutical Co.) by 5 concentrations (½, 1/2.5, ⅕, 1/10, and 1/20 of IC₅₀).

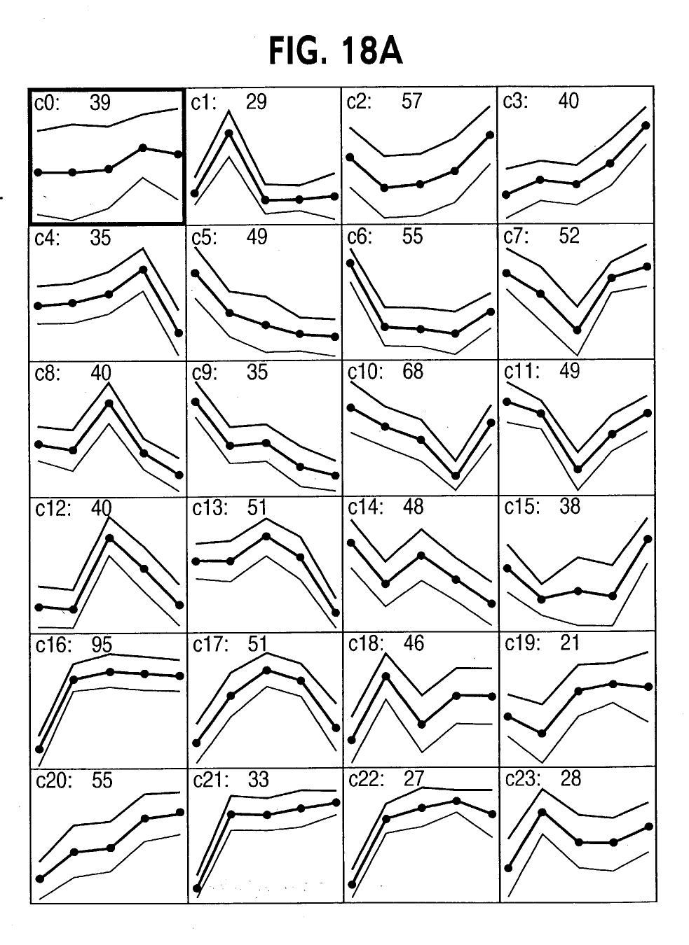

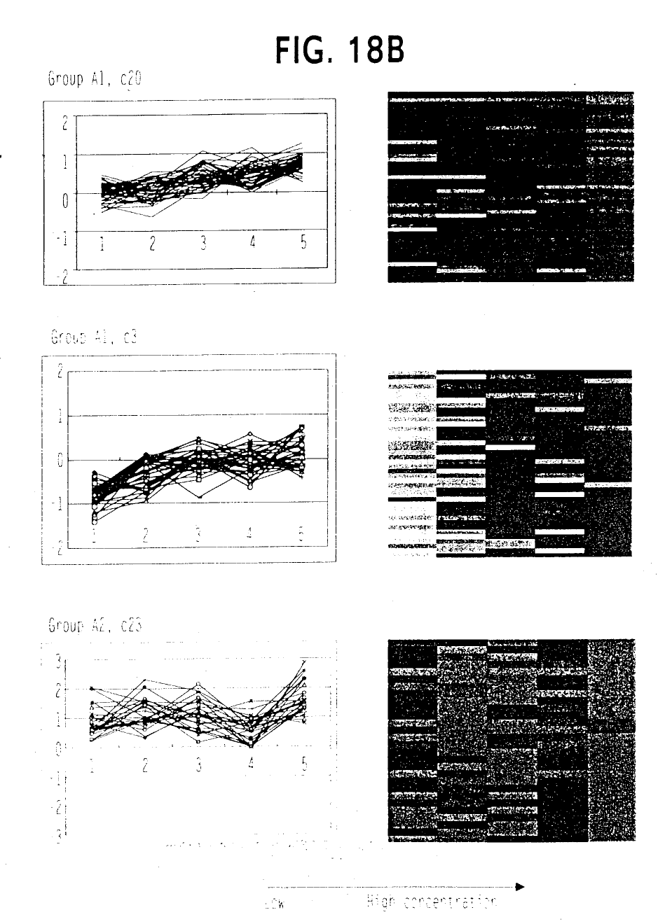

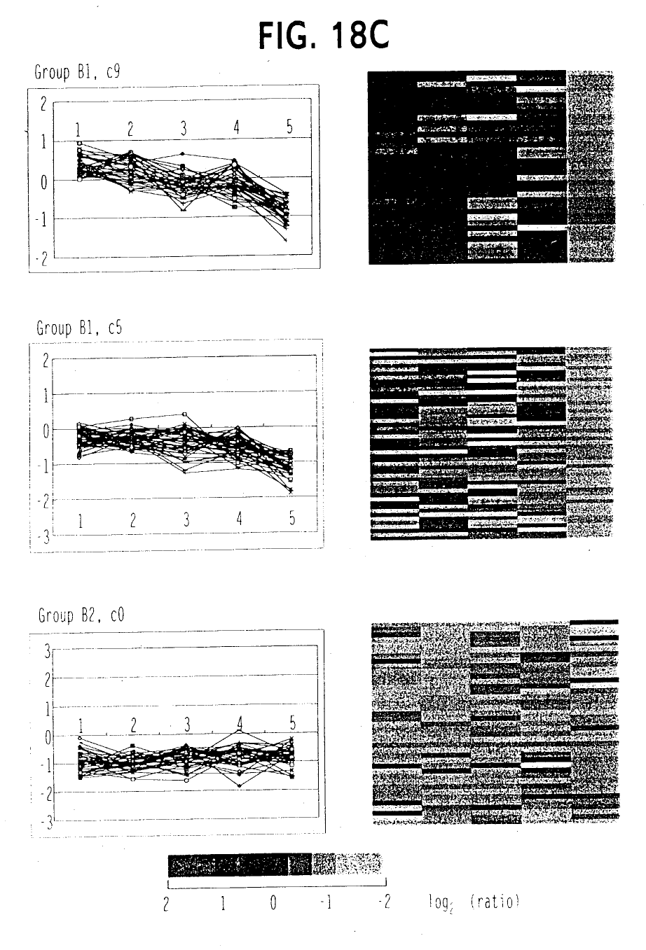

Nucleic Acid microarray with a two color detection method was employed to measure the expression profiles. The mRNA extracted from herbal treated cells was labeled with digoxigenin and the mRNA extracted from untreated cells was labeled with biotin. Arrays of 9600 features were employed and the procedures described by Chen et al. (Genomics, 51, 313-324, 1998) were adopted in the experiments. For data pre-processing, only array spots of high data quality were selected. The selection was based on signal to background ratio greater than 2.5 and 1.5× differential expression ratio. By these criteria, 1081 genes were selected for further statistical analysis. Non-hiearchical cluster analysis programs such as the Genecluster program developed in Massachusetts Institute of Technology (Tamayo et al., 1999) was employed to categorize the expression profiles. The Genecluster program is based on self organizing map (SOM) principle. A 6×4 SOM clustering of expression profiles are shown in FIG. 18A. The details of gene expression profiles for the selected clusters are shown in FIG. 18B. In these clusters, clusters 3 and 20 (labeled c3 and c20) were selected for that the gene expression levels increase with higher herbal concentrations. Similarly, clusters 5 and 9 for that the gene expression levels decrease with higher herbal concentrations. Cluster 23 collects genes whose expression levels were up regulated compared with that in untreated cells and cluster 0 for down regulated genes. These expression profile clusters are further condensed into two major groups, A & B. Group A collects genes up regulated by herbal treatment and Group B collects genes down regulated by herbal treatment. The expression profiles in Group A & B form the basis of a characteristic expression data set for an herbal preparation. The same procedures were repeated for 5 different batches of Huang Chin Tang, and 952 genes were selected to establish the characteristic expression profile database of the Huang Chin Tang.

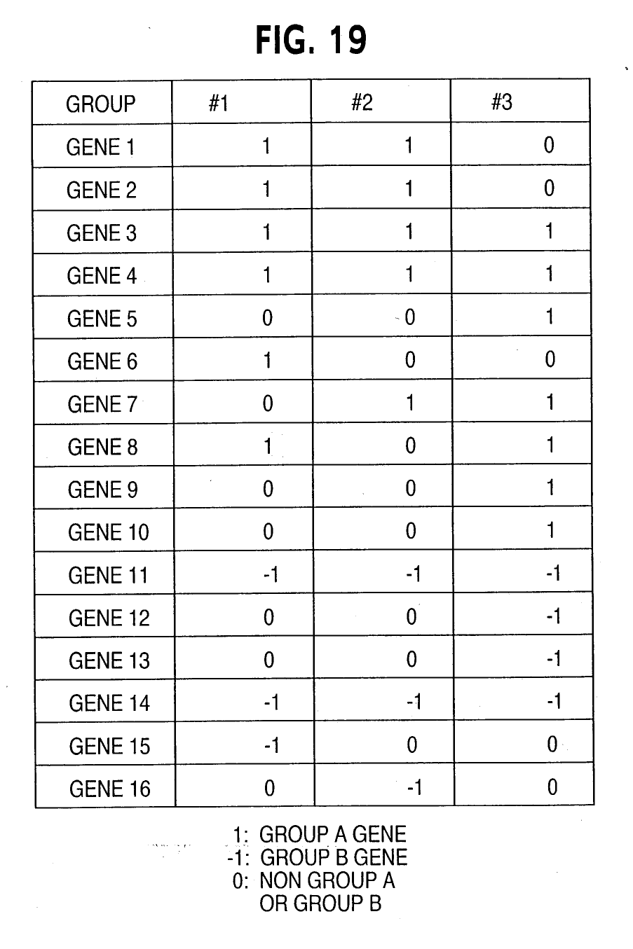

As shown in FIG. 19, by the aforementioned procedures, a gene can be categorized as Group A, B or none (non-A and non-B) and its expression profile can be represented by 1, −1, and 0 respectively. The number of different gene expression profiles between batch #1 and batch #2 are 3 in Group A (Gene 6, 7, and 8) and 2 in Group B (Gene 15 & 16). By the same principle, the number of different expression profiles between batch #1 and #3 are 10 in Group A and B and the number is 11 between batch #2 and batch #3. These numbers indicate that batch #1 and #2 are more similar than batch #3. This principle was applied to classify 5 different batches of herbal preparations. The following algorithm is designed to calculate the distance between a pair of herbal preparation batches, i and j.

dᵢⱼ=Σδ(Xᵢ, Xⱼ) (Hamming distance)

The gene X in i batch of preparation is assigned to Group A, B or none

if Xᵢ<>Xⱼ, δ(Xᵢ, Xⱼ)=1

if Xᵢ=Xⱼ, δ(Xᵢ, Xⱼ)=0

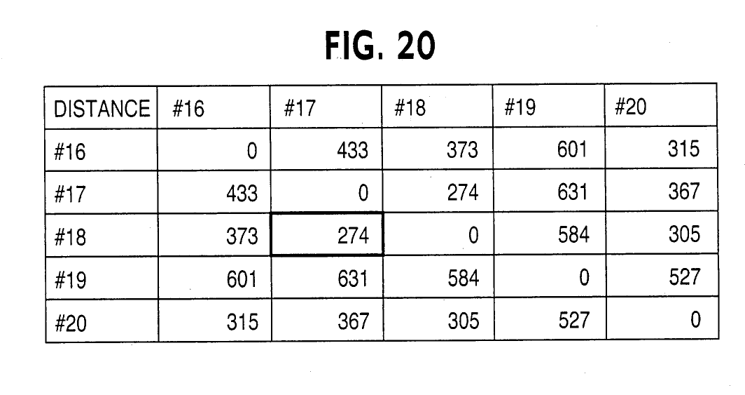

We calculated all the dᵢⱼ value between pairs of herbal preparations for cluster analysis. The analysis programs, Kitsch Cluster was based on hierarchical clustering principle and was written by Dr. Joseph Felsenstein of Washington University (http://evolution.genetics.washington.edu/phylip.html). From the Hamming distance table (FIG. 20), one can clearly identify that the shortest distance lie between batch #17 and batch #18 and that batch #17 is similar to #18. Batch #16 also similar with batch #17 and #18 but batch #19 is dissimilar to the rest of batches. The results were confirmed by HPLC analyses as described below.

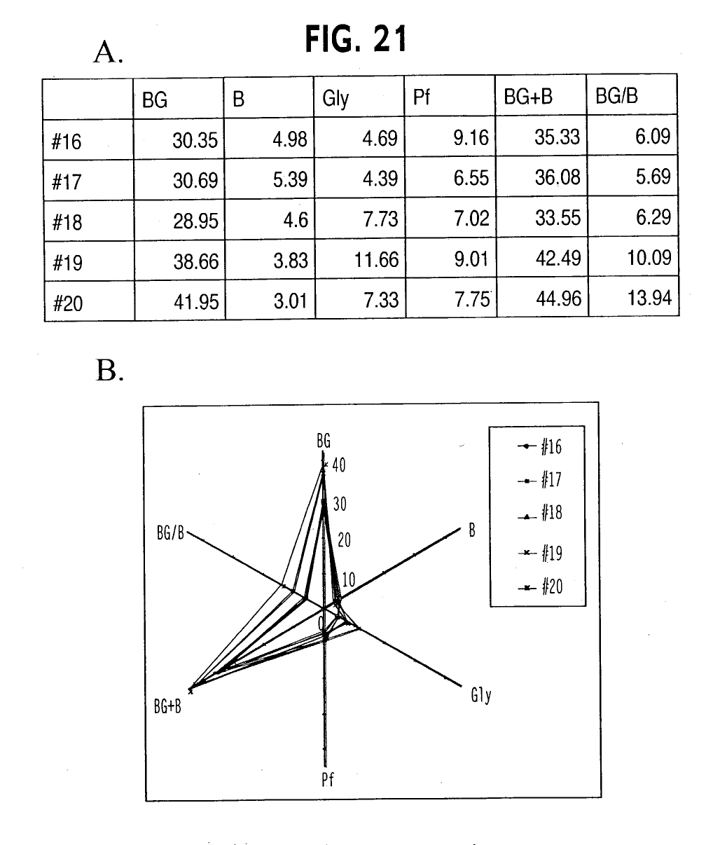

By HPLC, the chemical composition of the 5 batches of herbal preparations was analyzed. Four major peaks (BG, B, Gly, and Pf) in the chromatograms were selected for statistical analyses. Two additional parameters, BG+B and BG/B, were included in plotting the 6-coordinate radar graph as shown in FIG. 21. The distance on each coordinate is the integrated intensity of that particular chemical constituent in the chromatogram. In general, #16, #17 and #18 were similar in their constituent content (baicalin and baicalein) of Scutellariae Radix, which are within 33.55-36;08; while the amount of the same constituents are higher in #19 and #20 (42.49 and 44.96, respectively). The resemblance of #16, #17, and #18 can be seen from the coincident radar plots in FIG. 21B.

In order to identify the unknown herbal medicine based on the characteristic expression profile database established as described as above, Jurkat T cells were treated with a tester sample #17 in 5 concentrations to set up the characteristic expression data set for the tester. The Hamming distances between the tester and each of the data sets (#16, #17, #18, #19 and #20) in the characteristic expression database were calculated and the scores are: #16: 502, #17: 405, #18: 402, #19: 699, and #20: 531. These data show that the tester is most similar to #17 having a lowest Hamming distance score of 405. The example demonstrates that this invention teaches a method to identify unknown herbal medicine based on the gene expression profiles induced by the herbal medicine in mammalian cells. The identity of the unknown herbal medicine can be inferred by aligning the characteristic expression profiles with a collection of characteristic expression profiles of herbal medicines in an HBR Array.

Based on the characteristic expression database, marker genes and signature expression profiles can be deduced for an herbal medicine for studying its pharmacological mechanisms and for optimizing the formulation of a complex herbal preparation. For this example, 5 different batches of Huang Chin Tang preparations (#16, #17, #18, #19 and #20) were obtained from Sun Ten Pharmaceutical Co. and a characteristic expression profile database was constructed based on aforementioned procedures. For each gene, the consistency of expression profiles in the database was scored by the coefficient of variation (CV value):

CV=σ/(Σμᵢ/n)

μ: Mean expression ratios for #i treated cells.

n: Number of the data set, n=5 in this case.

σ: Standard deviation of the expression ratios for #16, #17, #18, #19 and #20.

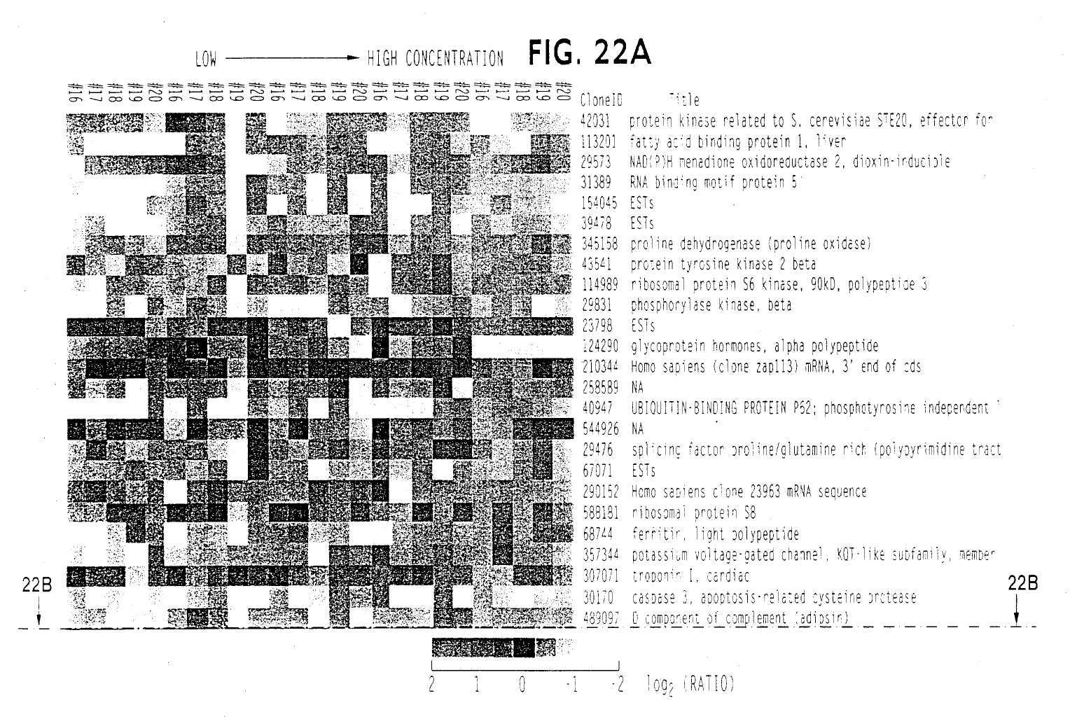

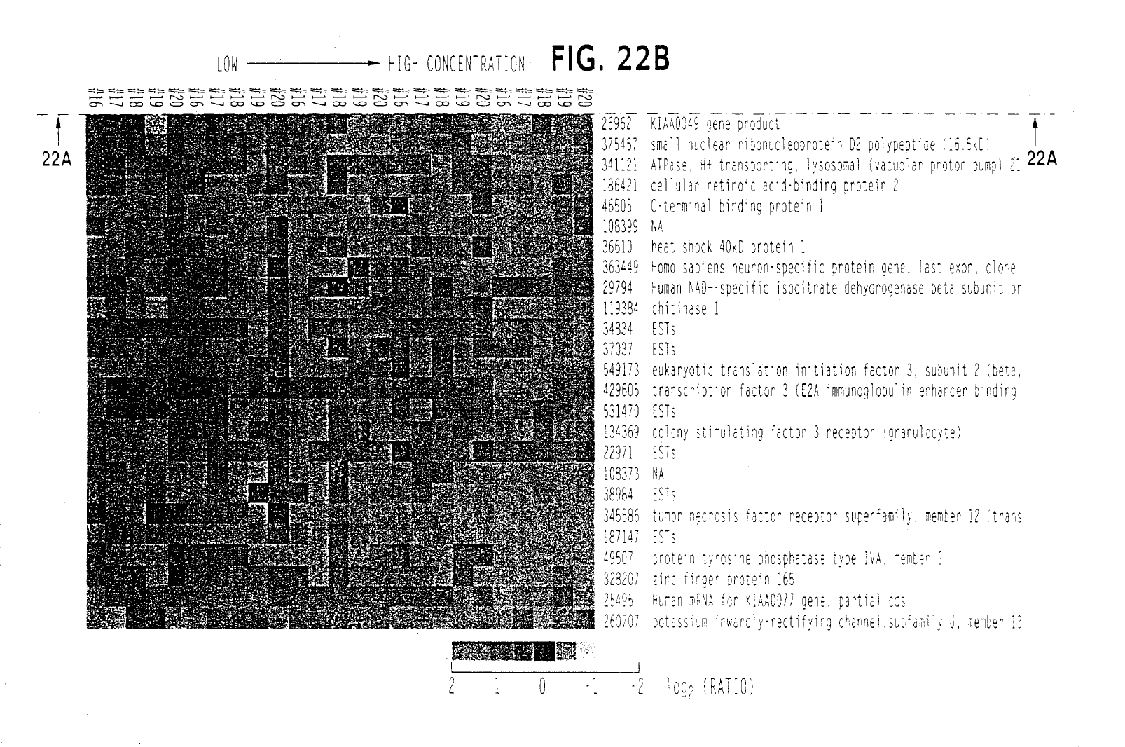

Since the CV reflects the variation of data, the marker genes for an herbal medicine were selected based on the CV score. The top 50 genes with the minimum CV scores were selected. FIG. 22 shows 25 market genes with up regulated signature profiles and 25 marker genes with down regulated signature profiles for Huang Chin Tang.

The characteristic expression profile database can be used to infer the expression profiles of individual chemical constituents in a mixture as complex as an herbal medicine if the amount of the chemical constituents can be semi-quantitatively determined. In this example, the chemical composition of an herbal medicine is determined by high performance liquid chromatography. The integrated intensities of 4 chemical constituents in five batches of Huang Chin Tang preparation were quantified by HPLC analysis. The gene expression ratios for each batch of herbal preparation were calculated by taking the median of the expression ratios induced by 5 concentrations of herbal preparation. The correlation between a constituent and a gene expression profile was quantified by the Pearson correlation coefficient. The Pearson correlation coefficient for gene x and the constituent y is:

R=(1/n)Σ(xᵢ−μₓ)(ᵢ−μ_{y})/σₓσ_{y}, i=1 to n

n: Number of the herbal preparation, n=5 in this case.

μₓ: Mean expression ratios in five herbal preparation for gene x.

μ_{y}: Mean integrated intensity in five batches of herbal preparation for constituent y.

xᵢ: Gene expression ratios in #i herbal preparation for gene x.

yᵢ: Integrated intensity in #i herbal preparation for constituent y.

σ: Standard deviation of the expression ratios (σₓ) or integrated intensities (σ_{y}) for five herbal preparations.

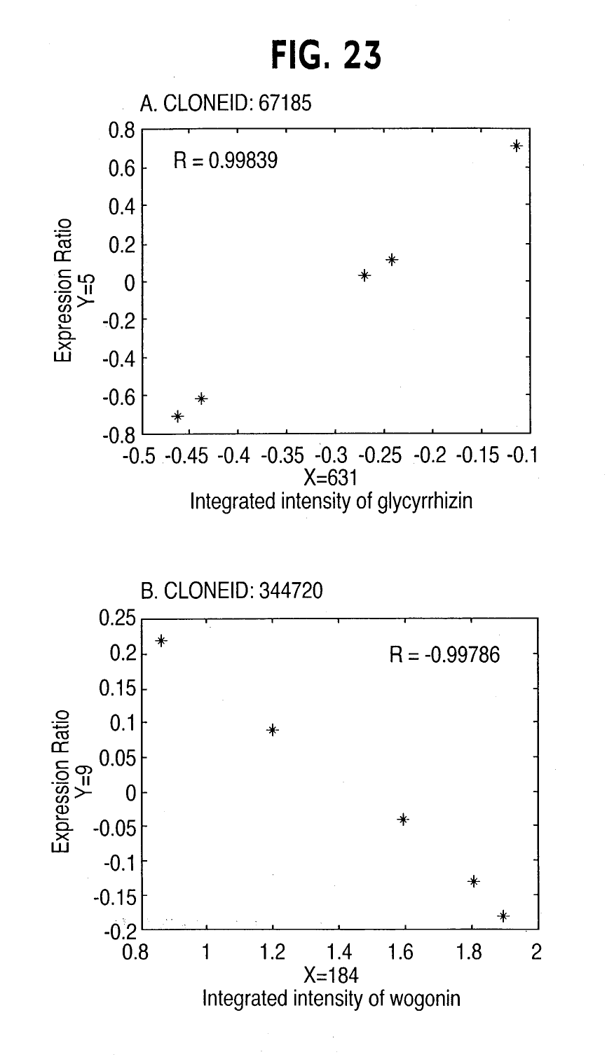

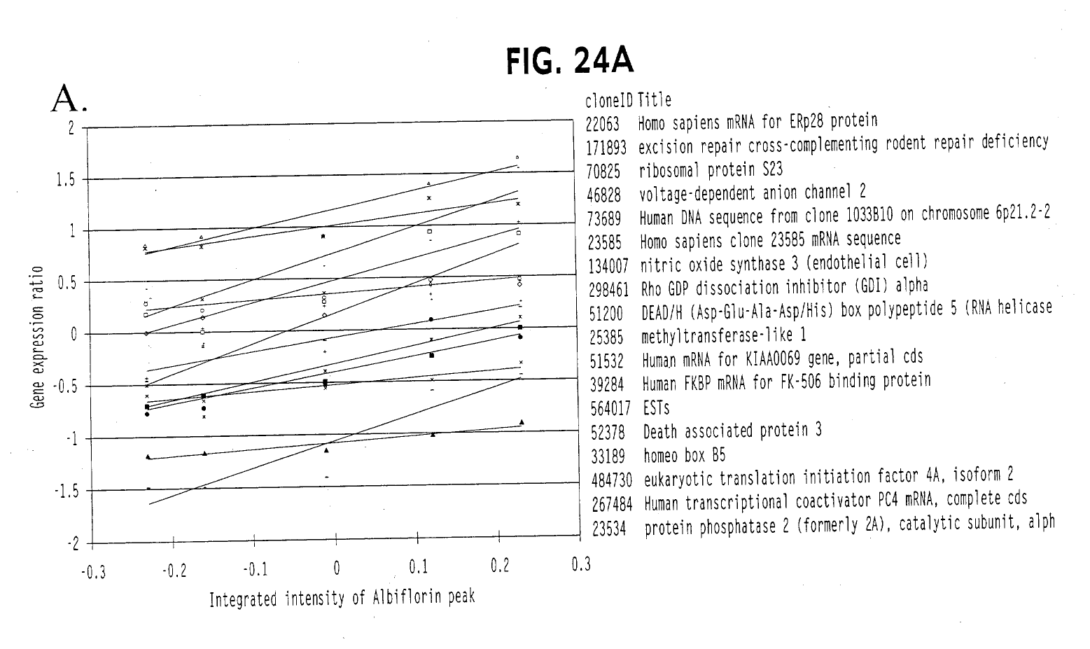

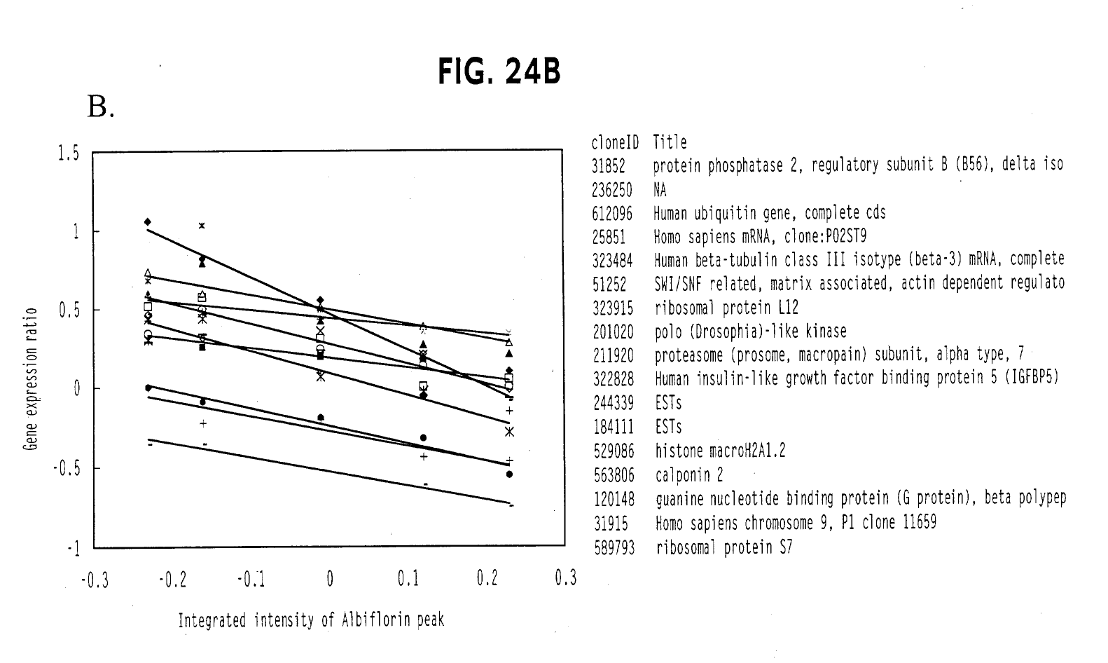

Several genes whose expression levels highly correlated (with |R|>0.99) with the amount of chemical constituents in Huang Chin Tang were identified for each constituent. For example, the R value between the gene, cloneID: 67185) and Glycyrrhizin was 0.998 (FIG. 23A). One the other hand, the gene (cloneID: 344720) whose expression levels increase with the decrease of Wogonin(WG) has an R value of −0.997 (FIG. 23B). In addition to the above two examples, 191 and 170 genes were highly correlated with individual constituents with R value >0.9 and R value <−0.9, respectively. For instance, 17 and 18 genes were positively and negatively, respectively, correlated with Albiflorin (Af) (FIG. 24). This example teaches a method to profile gene expression for individual constituents in a mixture without isolating them to perform the expression analyses one constituent by another.

### References for Example 16.

US Patent Documents

Stoughton-Roland and Friend-S H. USP #5965352: Methods for identifying pathways of drug action

Brown-P O and Shalon-T D USP #5807522: Methods for fabricating microarrays of biological samples

Lockhart-D J, Brown-E L, Wong,-G G, Chee-M S, and Gingeras-T R. USP #6040138: Expression monitoring by hybridization to high density oligonucleotide arrays

Ladunga-I. USP #5987390: Methods and systems for identification of protein classes

Mahant-S, Shivaling-S, Vivek-G. USP #5951711: Method and device for determining hamming distance between two multi-bit digital words

Foreign Patent Documents

Brown-P O and Shalon-T D EP #913485A1: Method and apparatus for fabricating microarrays of biological samples

Other Publications

Bertucci-F; Bernard-K; Loriod-B; Chang-Y C; Granjeaud-S; Birnbaum-D; Nguyen-C; Peck-K; Jordan-B R (1999) Sensitivity issues in DNA array-based expression measurements and performance of nylon microarrays for small samples Human Mol. Genet. 8(9): 1715-1722.

Bittner-L, Trent-J, Meltzer-P (1999) Data analysis and integration: of steps and arrows. Nature Genet. 22, 213-215.

Brown-P O; Botstein-D (1999) Exploring the new world of the genome with DNA microarrays. Nature genet. 21 (1) supplement, 33-37.

Chen-J J; Wu-R; Yang-P C; Huang-J Y; Sher-Y P; Han-M H; Kao-W C; Lee-P J; Chiu-T F; Chang-F; Chu-Y W; Wu-C W; Peck-K (1998) expression patterns and isolating differentially expressed genes by cDNA microarray; system with colorimetry detection. Genomics. 51: 313-24.

Chen-Y, Bittner-M, Dougherty-E R (1999) Issues associated with microarray data analysis and integration. Information supplementary to article by Michael Bittner, Jeffrey Trent and Paul Meltzer, Nature Genet. 22,-213-215.

Duggan-D J; Bittner-M; Chen-Y; Meltzer-P Trent -J M (1999) Expression profiling using cDNA microarrays. Nature genet. 21 (1) supplement, 10-14.

Eisen-M (1999) Cluster and Treeview manual. (ftp://rana.stanford.edu/software)

Eisen-M, Spellman-P T, Brown-P O, Botstein-D. (1998) Cluster analysis and display of gene-wide expression patterns. Proc. Natl. Acad. Sci. USA 99:14863-14868.

Golub-T R, Slonim-D K, Tamayo-P, Huard-C, Gaasenbeek-M, Mesirov-J P, Coller-H, Loh-M L, Downing-J R, Caligiuri-M A, Bloomfield-C D, and Lander-E S (1999) Molecular classification of cancer: class discovery and class prediction by gene expression monitoring. Science October 15: 531-537.

Lander-E S (1999) Array of hope. Nature genet., 21 (1) supplement, 34.

Tamayo-P, Slonim-D, Merirov-J, Zhu-Q, Kitareewan-S, Dmitrovsky-E, Lander-E S, and Golub-T R (1999) Interpreting patterns of gene expression with self-organizing maps: Methods and application to hematopoietic differentiation. Proc. Natl. Acad. Sci. USA 99:2907-2912.

Romesburg-H C (1989) Cluster analysis for researchers. Chapter 16: How to make classifications. P203-216, Krieger Publishing Co. Malabar, Fla., USA.

Scherf-U, Ross-D T, Waltham-M, Smith-L H, Lee-J K, Tanabe-L, Kohn-K W, Reinhold-W C, Myers-T G, Andrews-D T, Scudiero-D A, Eisen-M B, Sausville-E A, Pommier-Y, Botstein-D, Brown-P O, Weinstein-J N (2000) A gene expression database for the molecular pharmacology of cancer. Nature genet. 24: 23644.

Slonim-D K, Tamayo-P, Mesirov-J P, Golub-T R, Lander-E S (1999) Class prediction and discovery using gene expression data. (http://www.genome.wi.mit.edu/MPR).

Tavazole-S, Hughes-J D, Campbell-M J, Cho-R J, Church-G M. (1999) Systemic determination of genetic network architecture. Nature genet. 22: 281-285.

### Example 17

Identification of the Bioresponses and the Signature Genes of an Herbal Composition.

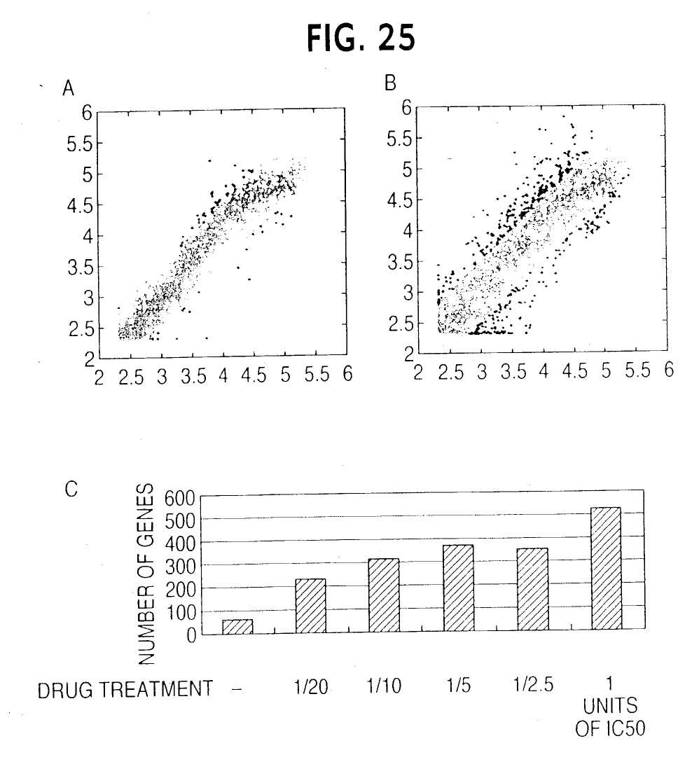

To further investigate the expression profiles induced by the Huang Chin Tang, Jurkat T cells were treated with Huang Chin Tang (PHY906 #2 obtained from Sun Ten Pharmaceutical Co. Taiwan) by 5 concentrations (1/20, 1/10, ⅕, 1/2.5, and 1 of IC₅₀). Nucleic acid microarray with dual-color detection was employed to measure the expression profiles. The mRNA extracted from herbal treated and untreated cells were labeled with Biotin-16-dUTP and Dig-11-dUTP, respectively. A control group was established by using mRNA extracted from untreated cells and labeled the mRNA with biotin and dig respectively in equal proportions. Five sample groups with different concentrations of herbal treatment and one control group were employed for the study by the procedures described by Chen et al. (1998) with minor modifications. For data pre-processing, only array spots of high data quality were selected. The selection criteria were spots with signal to background ratio greater than 2.5-fold and more than 1.5-fold in differential expression ratio. By these criteria, 1044 genes were selected for further statistical analysis. The gene expression profiles of the control group were highly correlated with only 48 genes listed as statistical outliers that lie beyond the 2-fold differential expression range (FIG. 25A). For the sample group, many differentially expressed genes are evident as illustrated in FIG. 25B. The number of genes whose differential expression ratio greater than 2-fold increases with concentration of herbal treatment as shown in FIG. 25C. These results prove that the identified differential expressed genes are truly induced by Huang Chin Tang treatments.

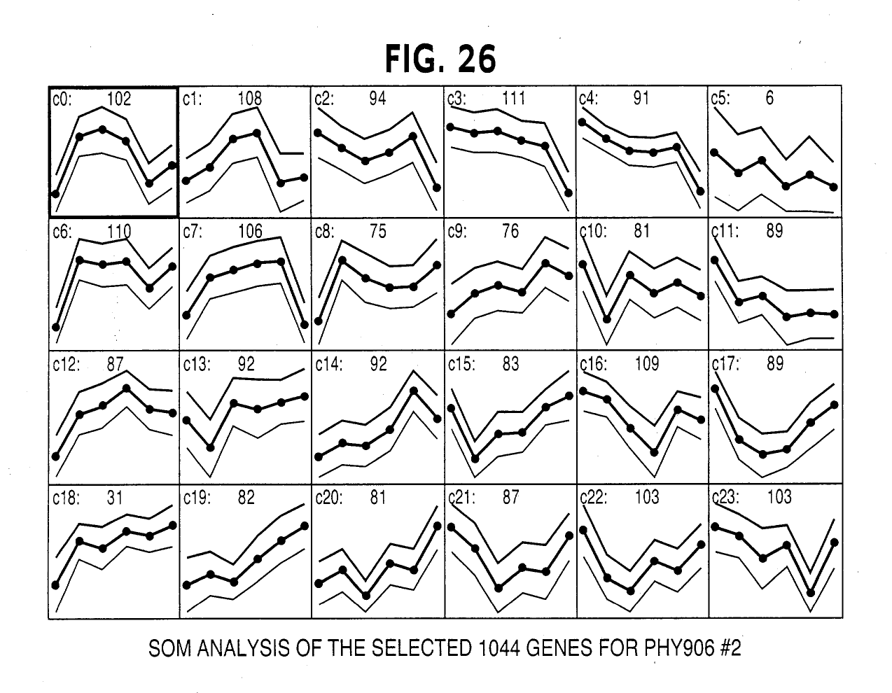

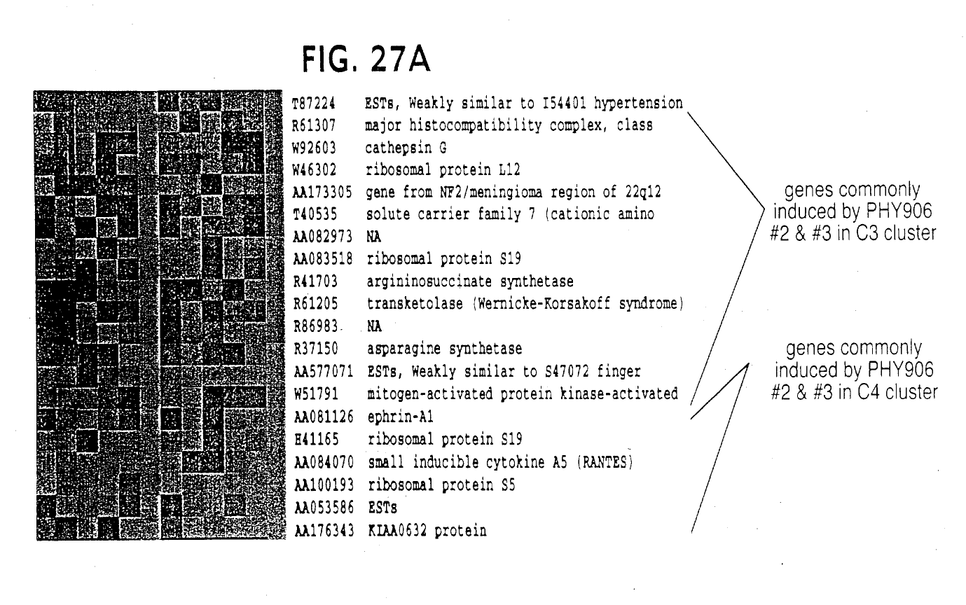

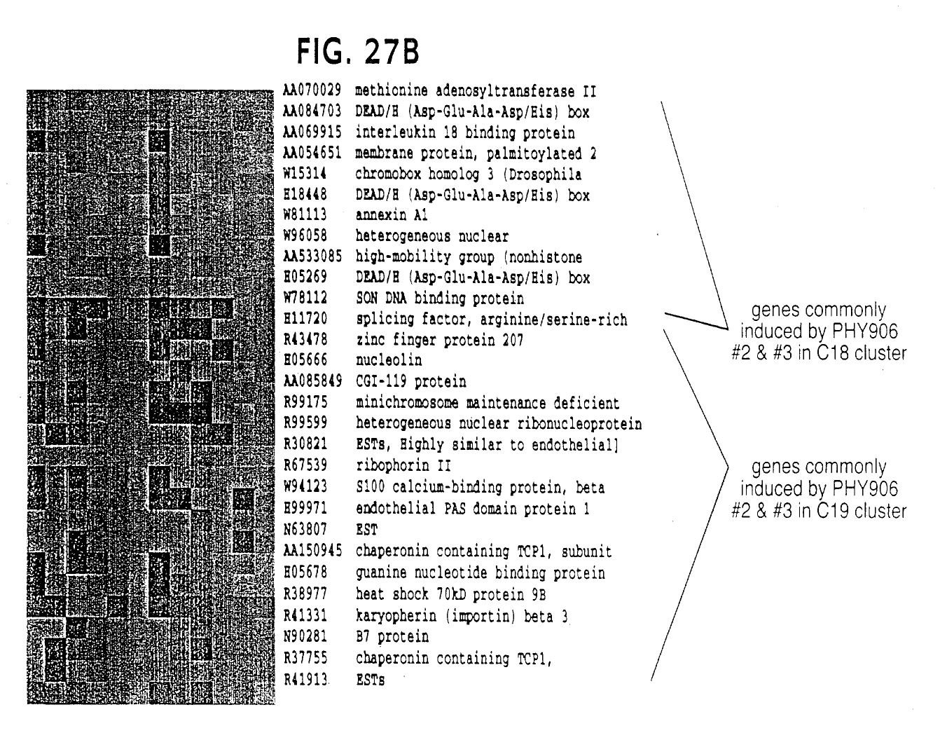

To identify the genes that were specifically induced by PHY906 (signature genes for PHY906), the expression profiles were clustered by a non-hierarchical cluster analysis programs “GeneCluster” developed by Tamayo et al., 1999. The computer program is based on self-organizing map (SOM) principle and the clusters of expression profiles are shown in FIG. 26. The X-axis represents the herbal concentration from low to high and the Y-axis is the gene-expression ratio. The signature genes were selected from the expression profiles which exhibit dosage response to the PHY906 #2. The induced and repressed genes were selected from cluster 3 & 4 and cluster 18 & 9, respectively. In order to identify signature genes for PHY906, another batch of Huang Chin Tang, PHY906 #3, with the same formula and manufacturing process were performed as described for PHY906 #2. The induced and repressed genes commonly found in both batches are shown in FIG. 27.

Score Similarity of Bioresponses by Self-Organizing Map (SOM)

To differentiate herbal medicines of similar compositions, a scoring method is developed and the score S represents the difference in bioresponses of a biosystem to two different herbal compositions.

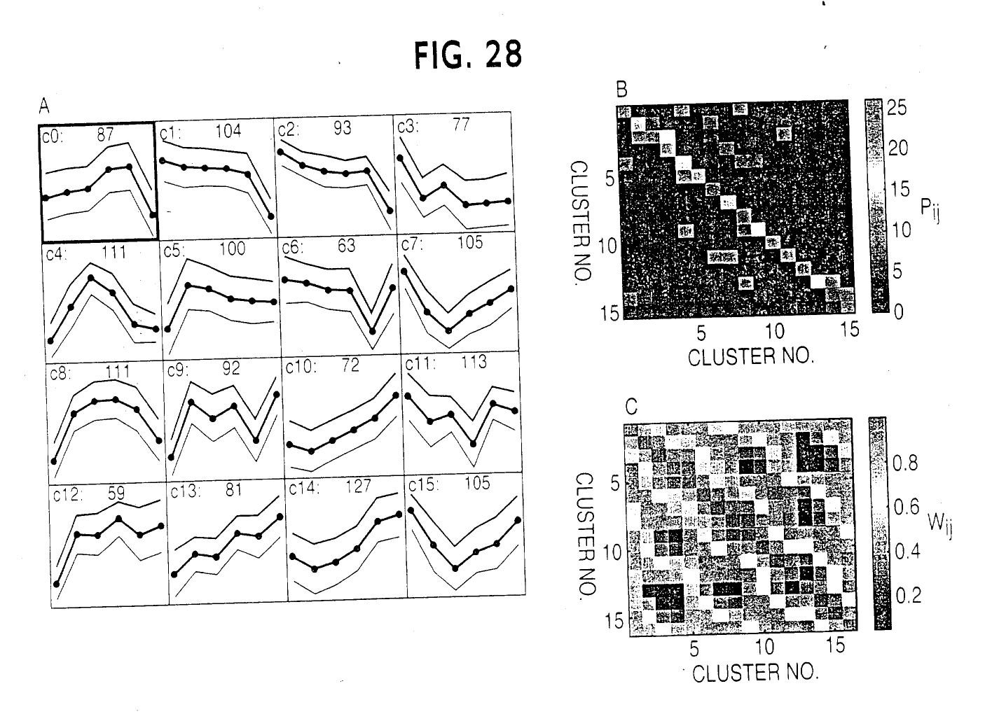

S=ΣPᵢⱼWᵢⱼ,

Where Pᵢⱼ is the number of the common genes induced both by herbal prep. A and herbal prep. B in cluster i and in cluster j. For example, the SOM clustering results for the expression profiles of both batches of PHY9066are shown in FIG. 28A. In cluster C13 and C14, 17 and 25 genes share the same expression profiles for both batches of PHY906, respectively. In addition, 10 genes whose expression profiles induced by PHY906 #2 are clustered in C13 but are clustered in C14 for PHY906 #3. Therefore, P₁₃₁₃=17, P₁₄₁₄=25, and P₁₃₁₄=10. A weighing factor, Wᵢⱼ, describes the distance between the cluster i and j to indicate the similarity of the two expression profile clusters. In the case of C13 and C14, these 10 genes have similarly response to PHY906 #2 and PHY906 #3 (FIG. 28B). The weighing factor is defined as:

Wᵢⱼ=1−Eᵢⱼ/Max(Eᵢⱼ), where Eᵢⱼ is the Euclidean distance between the cluster i and j and the value is normalized by Eᵢⱼ/Max(Eᵢⱼ). When i=j, Wᵢⱼ is 1. The number decreases as cluster i and cluster j become more different (FIG. 28C).

Classification of 5 Batches of Herbal Medicines

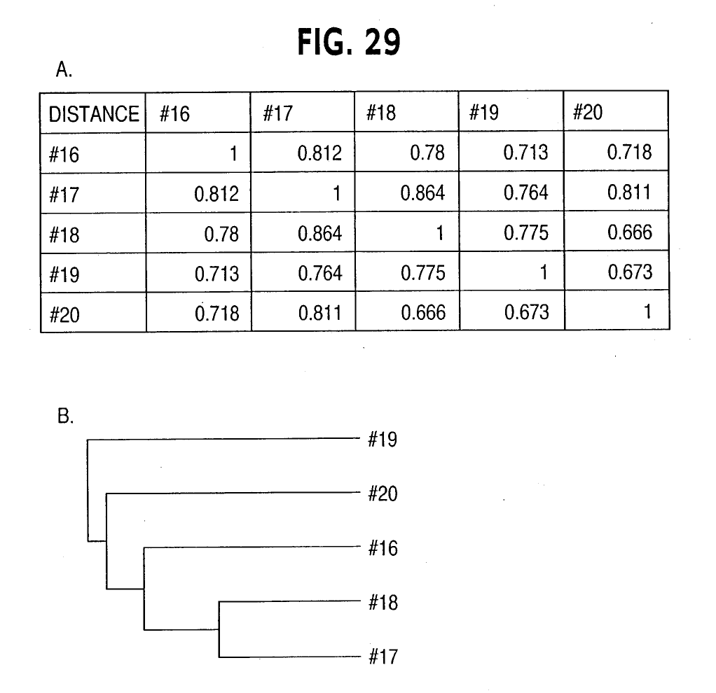

To test how well the above method performs in classifying 5 batches of similar herbal preparations, Jurkat T cells were treated with 5 batches of Huang Chin Tang (PHY01040; #16, PHY010402; #17, PHY03061; #18, PHY03062; #19 and PHY02231; #20 obtained from Sun Ten Pharmaceutical Co.) by 5 concentrations (1, 1/2.5, ⅕, 1/10, and 1/20 of IC₅₀). The Sᵢⱼ scores were calculated between pairs of herbal preparations in cluster analysis (FIG. 29). The analysis programs, Kitsch Cluster was based on hierarchical clustering principle and was written by Dr. Joseph Felsenstein of Washington University (http:/evolution.genetics.washington.edu/phylip.html). The S scores (distance) are tabulated (FIG. 29A), one can clearly identify that the shortest distance lie between batch #17 and batch #18 and that batch #17 is similar to #18. Batch #16 also similar with batch #17 and #18 but batch #19 is dissimilar to the rest of batches. The results were confirmed by HPLC analyses.

Characterize an Unknown Herbal Medicine Based on the Expression Profiles

To identify an unknown herbal medicine based on the characteristic expression profile database established as described as above, Jurkat T cells were treated with a tester sample #17 in 5 concentrations to set up the characteristic expression data set for the tester. The S score between the tester and each of the data sets (#16, #17, #18, #19 and #20) in the characteristic expression database were calculated and the S scores are: #16: 0.78, #17: 0.85, #18: 0.84, #19: 0.77, and #20: 0.79.These data show that the tester is most similar to #17 having a higher S score of 0.84. The example demonstrates that one can apply the method to identify an unknown herbal medicine based on the gene expression profiles induced by the herbal medicine in mammalian cells. The identity of the unknown herbal medicine can be inferred by aligning the characteristic expression profiles with a collection of characteristic expression profiles of herbal medicines in an HBR Array.

The property of an herb can be described by four natures and five flavors (in Chinese Herbal Pharmaceuticals, Ed. Zheng Hua Yen, People's Health publications, Beijing, China, 1997; Book of Ben Cao Gan Mu by Shi Zeng Li, Ming Dynasty, China). Each of the four herbs in PHY906 may relate to another set of herbs with similar property (see Table 19). Or herbs with similar property may exhibit similar bioresponse. HBR Arrays may be used to determine or measure the relatedness in terms of the property of herbs. Such information may be useful in creating a new herbal formulation.

TABLE 19
Herbs with PHY906 Properties

| Herb | Properties | Drugs with similar properties |
|---|---|---|
| Scutellariae | Bitter tasting, | Coptidis Rhizoma, |
| Radix | cold natured | Phellodendri Cortex, Gentianae Radix, |
| | | Gardeniae Fructus |
| Paeoniae | Bitter and sour | Canarii Fructus (sweet and |
| Radix | tasting, mildly | sour tasting, moderate natured), |
| | cold in nature. | Potulacae Herba (sour |
| | | tasting, cold natured), |
| | | Fraxini Cortex(bitter tasting, |
| | | cold natured), Sophorae |
| | | Flos (bitter tasting, mild |
| | | cold in nature), Bletillae Tuber |
| | | (bitter, sweet and harsh in taste, |
| | | and mildly cold in nature). |
| Glycyrrhizae | Sweet tasting, | Lycii Fructus, Polygonati |
| Radix | moderate natured | Officinalis Rhizoma, |
| | | Polygonati Rhizoma |
| Zizyphi | Sweet tasting and | Saccharum Granorum, Juglandis Semen |
| Fructus | warm natured | |

Properties:

Four natures-cold, hot, warm, cool.

Five flavors-acrid, bitter,, sweet, sour, bland.

### Example 18

Evaluation of an Herbal Medicines by HBR Array.

As stated in Example 16, the component herbs of Huang Chin Tang are Scute, Peony, Licorice and Jujube. The gene expression profiles induced by five batches of Huang Chin Tang in mammalian cells were characterized. A standard formula for Huang Chin Tang can be defined and characterized with animal studies or with clinical studies. For example, the #17 was used as the standard formula for Huang Chin Tang based on the quality control and other standards set up by Sun Ten Pharmaceutical Co. The bioresponses of #17 were used to build the HBR Array for Huang Chin Tang. The marker genes in the HBR Array were selected to evaluate other preparations of Huang Chin Tang composition. A tester Huang Chin Tang may contain the same herb compositions but the component herbs may be grown under various environmental characteristics. Comparing the bioresponses of the tester with the marker genes of standardized HBR Array, the biological activities of the tester were evaluated.

Furthermore, the genes whose expression levels are highly correlated (with |R|>0.99) with the dosage of component herbs in Huang Chin Tang (as stated in Example 16 and FIG. 25) are selected for evaluation purpose. The tester Huang Chin Tang can be evaluated by comparing the specific bioresponses or expression levels of the selected set of marker genes with the HBR Array. If the expression levels or bioresponses of the selected marker genes are beyond the acceptable variation region, the amount or characteristics of the component herbs are adjusted or modified to meet the acceptable variation. The process is repeated until the bioresponses induced by the revised herbal composition are within the acceptable variation range by comparing with the standard HBR Array.

### Example 19

Predicting Biological Activity and Therapeutic Applications of an Herbal Composition.

According to the identified marker genes for PHY 906 (FIG. 27), these genes can be used to predict the biological activities of the herbal composition. For example, the following underlined marker genes of PHY906 have been reported to involve in the following biological activities and therapeutic effects. The only effective drug against ALL is to inhibit the asparagine synthetase due to increased cellular apoptosis (Nandy et al., 1998). Long-acting drug somatostatin analogs are applied in the treatment of neurofibroma for their tumor growth inhibitory effect because they induce antiproliferative action mediated by the inhibition of G6PD, transketolase, or both (Boros et al., 1998). Ephrin-A1 is a new melanoma growth factor and is highly expressed during melanoma progression (Easty et al., 1999). Mitogen-activated protein kinase (MAPK) family members have been recently reported to have opposing effects on apoptosis (Dabrowski et al., 2000). The expressions of asparagine synthetase, transketolase, ephrin-A1 and MAPK are repressed with the higher concentration of PHY906 treatments. The down-regulation of these genes are involved in cell apoptosis. The expression of the enzyme argninosuccinate synthetase, cathepsin G and chemokine RANTES are highly induced in inflammatory mechanism. By the PHY906 treatment the inflammatory involved genes are suppressed. These literature reports provide a basis for predicting the biological activities or therapeutic effects of an herbal composition.

### References for Example 19

Nandy-P; Periclou-A P; Avramis-V I (1998) The synergism of 6-mereapopurine plus cytosine arabinoside followed by PEG-asparaginase in human leukemia cell lines (CCRF/CEM/0 and CCRF/CEM/ara-C/7A) is due to increased cellular apoptosis. Anticancer Research 18: 727-737.

Boros-L G; Brandes-J L; Yusuf-F I; Cascante-M; Williams-R D; Schirmer-W J (1998) Inhibition of the oxidative and nonoxidative pentose phosphate pathways by somatostatin: a possible mechanism of antitumor action. Medical Hypotheses 50: 501-506.

Easty-D J; Hill-S P; Hsu-M Y; Fallowfield-M E; Florenes-V A; Herlyn-M; Benett-D C (1999) Up-regulation of ephrin-A1 during melanoma progression. Int. J. Cancer 84:494-501.

Dabrowski-A; Tribillo-I; Dabrowska M I; Wereszczynska-S U; Gabryelewicz-A (2000) Activation of mitogen-activated protein kinases in different models of pancreatic acinar cell damage. Z-Gastroenterol. 38: 469-481.

The foregoing detailed description has been given for clearness of understanding only and no unnecessary limitations should be understood therefrom as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A method of establishing a standardized Herbal BioResponse Array (HBR Array) for an herbal composition comprising:
d) selecting a characterized herbal composition;
e) exposing a biosystem to a batch of the characterized herbal composition and collecting data on two or more markers, wherein one of the markers is a change in gene expression determined through the use of a nucleic acid microarray, produced by the steps comprising:
iv) producing a cell banking system;
v) profiling the gene expression pattern of cells from the cell banking system before and after exposure to the herbal composition;
vi) selecting as markers those genes whose expression levels are changed by exposure to the herbal composition;
f) storing the marker data of step b) as a standardized HBR array.

2. The method of claim 1, further comprising:
g) repeating steps b) and c) for one more batches of the herbal composition using two or more of the same or different markers than used in step b);
h) combining the HBR Arrays obtained in steps c) and d); and
i) analyzing the combined HBR Array of step e) to generate a standardized HBR Array for the characterized herbal composition.

3. The method of claims 1 or 2, wherein the characterized herbal composition has at least one known BioResponse.

4. The method of claims 1 or 2, wherein one or more of the following is known for the characterized herbal composition: chemical testing, the part of the plant used, the growing conditions of one or more of the individual herbs in the characterized herbal composition, the pre-harvest treatment of one or more of the individual herbs in the characterized herbal composition, the post-harvest treatment of one or more of the individual herbs in the characterized herbal composition, the post-harvest treatment of the characterized herbal composition, and the relative proportions of the individual herbs in the herbal composition.

5. The method of claims 1 or 2, wherein the cell banking system comprises a master cell bank and a working cell bank.

6. The method of claim 5, wherein the cells of the working cell bank are obtained from the master cell bank.

7. The method of claim 5, wherein the step of profiling the gene expression pattern of cells from the cell banking system before and after exposure to the herbal composition is performed using cells from the working cell bank.

8. The method of claims 1 or 2, wherein the change in gene expression is determined using a nucleic acid microarray.

9. The method of claim 8, wherein the said genes whose expression levels are changed by exposure to the herbal composition are selected based on the criteria of having a signal to noise ratio of about 2.5 or greater in the nucleic acid microarray and having an about 1.5 or greater change in the differential expression ratio.

10. The method of claim 8, wherein data regarding between about 10 and about 20,000 genes whose expression levels are changed is stored as part of the HBR Array.

11. The method of claim 10, wherein data between about 10 and about 1,500 genes whose expression levels are changed is stored as part of the HBR Array.

12. A method of evaluating an herbal composition comprising:
a) exposing a biosystem to a batch of the herbal composition and collecting data on two or more markers, wherein one of the markers is a change in gene expression determined through the use of a nucleic acid microarray, produced by the steps comprising:
i) producing a cell banking system;
ii) profiling the,gene expression pattern of cells from the cell banking system before and after exposure to the herbal composition;
iii) selecting as markers those genes whose expression levels are changed by exposure to the herbal composition;
b) comparing the collected marker data with a standardized HBR Array for the same or a substantially same herbal composition as that of the batch herbal composition, wherein the standardized HBR Array contains one of the markers data on gene expression.

13. A method of determining if an herbal composition meets a standard specification comprising:
a) exposing a biosystem to a batch of the herbal composition and collecting data on two or more markers, wherein one of the markers is a change in gene expression determined through the use of a nucleic acid microarray, produced by the steps comprising:
i) producing a cell banking system;
ii) profiling the genie expression pattern of cells the cell banking system before and after exposure to the herbal composition;
iii) selecting as markers those genes whose expression levels are changed by exposure to the herbal composition;
b) comparing the collected marker data with a standardized HBR Array for the same or a substantially same herbal composition as that of the batch herbal composition, wherein the standardized HBR Array contains as one of the markers data on gene expression; and
c) determining which herbal compositions have marker data that is similar to that of the standardized HBR Array within an acceptable level.

14. The method of claim 113, wherein said determining which herbal compositions have marker data which is similar to that of the standardized HBR Array within an acceptable level is determined quantitatively or qualitatively.

15. The method of claims 13 or 14, wherein the standardized HBR Array includes an acceptable range of variation for each marker.

16. A method of adjusting the components of a herbal composition so that it meets a standard specification for the same or substantially the same herbal composition, comprising:
a) exposing a biosystem to a batch of the herbal composition and collecting data on two or more markers, wherein one of the markers is a change in gene expression determined through the use of a nucleic acid microarray, produced by the steps comprising:
i) producing a cell banking system;
ii) profiling the gene expression pattern of cells the cell banking system before and after exposure to the herbal composition;
iii) selecting as markers those genes whose expression levels are changed by exposure to the herbal composition;
b) comparing the collected marker data with a standardized HBR Array for the same or a substantially same herbal composition as that of the batch herbal composition, wherein the standardized HBR Array contains as one of markers data on gene expression, and wherein the standardized HBR Array also includes an acceptable range of variation for each marker;
c) determining whether the herbal composition has marker data that is within the acceptable level of variation for the standardized HBR Array; and
d) if the marker data is not within the acceptable level of variation for the standardized HBR Array, adjusting the components of the herbal composition.

17. The method of claim 16, wherein steps (a) through (d) are repeated until the marker data of the herbal composition is within the acceptable level of variation of the standardized HBR Array.

18. A method of changing the components of a herbal composition so that it meets a standard specification of another herbal composition, comprising:
a) exposing a biosystem to a batch of the herbal composition and collecting data on two or more markers, wherein one of the markers is a change in gene expression determined through the use of a nucleic acid microarray, produced by the steps comprising:
i) producing a cell banking system;
ii) profiling the gene expression pattern of cells the cell banking system before and after exposure to the herbal composition;
iii) selecting as markers those genes whose expression levels are changed by exposure to the herbal composition;
b) comparing the collected marker data with a standardized HBR Array for the other herbal compositions as that of the batch herbal composition, wherein the standardized HBR Array contains as one of markers data on gene expression, and wherein the standardized HBR Array also includes an acceptable range of variation for each marker;
c) determining whether the herbal composition has marker data that is within the acceptable level of variation for the standardized HBR Array; and
d) if the marker data is not within the acceptable level of variation for the standardized HBR Array, changing the components of the herbal composition.

19. The method of claim 18, wherein steps a) through d) are repeated until the marker data of the herbal composition is within the acceptable level of variation of the standardized HBR Array.

20. A method for predicting the biological activity of an herbal composition comprising:
a) exposing a biosystem to a batch of the herbal composition and measuring the differential responses of two or more markers, wherein one of the markers is a change in gene expression determined through the use of a nucleic acid microarray, produced by the steps comprising;
i) producing a cell banking system;
ii) profiling the gene expression pattern of cells the cell banking system before and after exposure to the herbal composition;
iii) selecting as markers those genes whose expression levels are changed by exposure to the herbal composition;
wherein the set of differential response measurements constitute an Herbal BioResponse Array (HBR Array) data set;
b) comparing the HBR Array of the batch herbal composition to at least one previously-obtained HBR Array of a characterized herbal composition, wherein the previously-obtained HBR Array contains as one of the markers data on gene expression; and
c) predicting the biological activity of the batch herbal composition based on the HBR Array comparison made in step b).

21. A method of measuring the relatedness of a herbal composition to a characterized herbal composition comprising:
a) exposing a biosystem to a batch of the herbal composition and measuring the differential responses of two or more markers, wherein one of the markers is a change in gene expression determined through the use of a nucleic acid microarray, produced by the steps comprising;
i) producing a cell banking system;
ii) profiling the gene expression pattern of cells the cell banking system before and after exposure to the herbal composition;
iii) selecting as markers those genes whose expression levels are changed by exposure to the herbal composition;
wherein the set of differential response measurements constitute an Herbal BioResponse Array (HBR Array) data set;
b) comparing the HBR Array of the batch herbal composition to at least one previously-obtained HBR Array of a characterized herbal composition, wherein the previously-obtained HBR Array contains as one of the markers data on gene expression; and
c) determining the relatedness of the herbal composition to the characterized herbal composition based on the HBR Array comparison made in step b).

22. A method for predicting new therapeutic applications of an herbal comprising:
a) exposing a biosystem to a batch of the herbal composition and measuring the differential responses of two or more markers, wherein one of the markers is a change in gene expression determined through the use of a nucleic acid microarray, produced by the steps comprising;
i) producing a cell banking system;
ii) profiling the gene expression pattern of cells the cell banking system before and after exposure to the herbal composition;
iii) selecting as markers those genes whose expression levels are changed by exposure to the herbal composition;
wherein the set of differential response measurements constitute an Herbal BioResponse Array (HBR Array) data set;
b) predicting the new therapeutic applications based on the predicted biological activity of the markers in the HBR Array.

23. A method for determining the gene expression profile induced by individual chemical entities in an herbal composition comprising:
a) producing a cell banking system;
b) profiling the gene expression pattern of cells from the cell banking system before and after exposure to the herbal composition;
c) selecting as markers those genes whose expression levels are changed by exposure to the herbal composition and placing into an HBR Array;
d) comparing the HBR Array generated in step (c) with a standardized HBR Array for a similar or modified herbal composition;
e) determining the relative amounts of the individual chemical entities of the herbal composition; and
f) comparing the amount of the individual chemical entities to the result of step (b) to identify those genes whose expression levels change as the amount of the individual chemical entity in the herbal composition changes.

24. A method for determining the gene expression profile induced by individual chemical entities in a complex mixture without extracting the chemicals from the complex mixture such as an herbal composition comprising
a) producing a cell banking system;
b) profiling the gene expression pattern of cells from the cell banking system before and after exposure to the herbal composition;
c) selecting as markers those genes whose expression levels are changed by exposure to the herbal composition;
d) comparing the collected marker data with a standardized HBR Array for a substantially same or modified herbal composition;
e) characterizing the chemical components of the said herbal compositions;
f) comparing the identified chemical compositions to identify the differential levels of individual chemical components in herbal compositions;
g) correlating the differential chemical component amounts with the differential bioresponses in the HBR Array to identify the characteristic bioresponses for each chemical entity.
